SEQUENCE LISTING (233 sequences)

SEQ ID NO: 1 - RNA, 83 nt, Artificial Sequence
  Description of Artificial Sequence Synthetic oligonucleotide
ccuuggaguaaaguagcagcacauaaugguuuguggauuuugaaaaggugcaggccauauugugcugccucaaaaauacaagg

SEQ ID NO: 2 - RNA, 22 nt, Artificial Sequence
  Description of Artificial Sequence Synthetic oligonucleotide
uagcagcacauaaugguuugug

SEQ ID NO: 3 - RNA, 89 nt, Artificial Sequence
  Description of Artificial Sequence Synthetic oligonucleotide
gucagcagugccuuagcagcacguaaauauuggcguuaagauucuaaaauuaucuccaguauuaacugugcugcugaaguaagguugac

SEQ ID NO: 4 - RNA, 22 nt, Artificial Sequence
  Description of Artificial Sequence Synthetic oligonucleotide
uagcagcacguaaauauuggcg

SEQ ID NO: 5 - RNA, 22 nt, Artificial Sequence
  Description of Artificial Sequence Synthetic oligonucleotide
aagcugccaguugaagaacugu

SEQ ID NO: 6 - RNA, 85 nt, Artificial Sequence
  Description of Artificial Sequence Synthetic oligonucleotide
ggcugagccgcaguaguucuucaguggcaagcuuuauguccugacccagcuaaagcugccaguugaagaacuguugcccucugcc

SEQ ID NO: 7 - RNA, 22 nt, Artificial Sequence
  Description of Artificial Sequence Synthetic oligonucleotide
uucaaguaauccaggauaggcu

SEQ ID NO: 8 - RNA, 77 nt, Artificial Sequence
  Description of Artificial Sequence Synthetic oligonucleotide
guggccucguucaaguaauccaggauaggcugugcaggucccaaugggccuauucuugguuacuugcacggggacgc

SEQ ID NO: 9 - RNA, 84 nt, Artificial Sequence
  Description of Artificial Sequence Synthetic oligonucleotide
ggcuguggcuggauucaaguaauccaggauaggcuguuuccaucugugaggccuauucuugauuacuuguuucuggaggcagcu

SEQ ID NO: 10 - RNA, 64 nt, Artificial Sequence
  Description of Artificial Sequence Synthetic oligonucleotide
augacugauuucuuuugguguucagagucaauauaauuuucuagcaccaucugaaaucgguuau

SEQ ID NO: 11 - RNA, 22 nt, Artificial Sequence
  Description of Artificial Sequence Synthetic oligonucleotide
uagcaccaucugaaaucgguua

SEQ ID NO: 12 - RNA, 23 nt, Artificial Sequence
  Description of Artificial Sequence Synthetic oligonucleotide
uagcaccauuugaaaucaguguu

SEQ ID NO: 13 - RNA, 81 nt, Artificial Sequence
  Description of Artificial Sequence Synthetic oligonucleotide
cuucaggaagcugguuucauauggugguuuagauuuaaauagugauugucuagcaccauuugaaaucaguguucuuggggg

SEQ ID NO: 14 - RNA, 81 nt, Artificial Sequence
  Description of Artificial Sequence Synthetic oligonucleotide
cuucuggaagcugguuucacaugguggcuuagauuuuuccaucuuuguaucuagcaccauuugaaaucaguguuuuaggag

SEQ ID NO: 15 - RNA, 22 nt, Artificial Sequence
  Description of Artificial Sequence Synthetic oligonucleotide
uagcaccauuugaaaucgguua

SEQ ID NO: 16 - RNA, 88 nt, Artificial Sequence
  Description of Artificial Sequence Synthetic oligonucleotide
aucucuuacacaggcugaccgauuucuccugguguucagagucuguuuuugucuagcaccauuugaaaucgguuaugauguaggggga

SEQ ID NO: 17 - RNA, 22 nt, Artificial Sequence
  Description of Artificial Sequence Synthetic oligonucleotide
uguaaacauccuugacuggaag

SEQ ID NO: 18 - RNA, 92 nt, Artificial Sequence
  Description of Artificial Sequence Synthetic oligonucleotide
gggcagucuuugcuacuguaaacauccuugacuggaagcuguaagguguucagaggagcuuucagucggauguuuacagcggcaggcugcca

SEQ ID NO: 19 - RNA, 23 nt, Artificial Sequence
  Description of Artificial Sequence Synthetic oligonucleotide
uguaaacauccuacacucucagc

SEQ ID NO: 20 - RNA, 89 nt, Artificial Sequence
  Description of Artificial Sequence Synthetic oligonucleotide
accaugcuguaguguguguaaacauccuacacucucagcugugagcucaagguggcugggagaggguuguuuacuccuucugccaugga

SEQ ID NO: 21 - RNA, 77 nt, Artificial Sequence
  Description of Artificial Sequence Synthetic oligonucleotide
ccgggacccaguucaaguaauucaggauagguugugugcuguccagccuguucuccauuacuuggcucggggaccgg

SEQ ID NO: 22 - RNA, 21 nt, Artificial Sequence
  Description of Artificial Sequence Synthetic oligonucleotide
uucaaguaauucaggauaggu

SEQ ID NO: 23 - RNA, 72 nt, Artificial Sequence
  Description of Artificial Sequence Synthetic oligonucleotide
agauacuguaaacauccuacacucucagcuguggaaaguaagaaagcugggagaaggcuguuuacucuuucu

SEQ ID NO: 24 - RNA, 22 nt, Artificial Sequence
  Description of Artificial Sequence Synthetic oligonucleotide
cugggagaaggcuguuuacucu

SEQ ID NO: 25 - RNA, 110 nt, Artificial Sequence
  Description of Artificial Sequence Synthetic polynucleotide
ggccagcugugaguguuucuuuggcagugucuuagcugguuguugugagcaauaguaaggaagcaaucagcaaguauacugcccuagaagugcugcacguuguggggccc

SEQ ID NO: 26 - RNA, 22 nt, Artificial Sequence
  Description of Artificial Sequence Synthetic oligonucleotide
uggcagugucuuagcugguugu

SEQ ID NO: 27 - RNA, 22 nt, Artificial Sequence
  Description of Artificial Sequence Synthetic oligonucleotide
ugagaacugaauuccauggguu

SEQ ID NO: 28 - RNA, 99 nt, Artificial Sequence
  Description of Artificial Sequence Synthetic oligonucleotide
ccgauguguauccucagcuuugagaacugaauuccauggguugugucagugucagaccucugaaauucaguucuucagcugggauaucucugucaucgu

SEQ ID NO: 29 - RNA, 22 nt, Artificial Sequence
  Description of Artificial Sequence Synthetic oligonucleotide
ucucccaacccuuguaccagug

SEQ ID NO: 30 - RNA, 84 nt, Artificial Sequence
  Description of Artificial Sequence Synthetic oligonucleotide
cuccccauggcccugucucccaacccuuguaccagugcugggcucagacccugguacaggccugggggacagggaccuggggac

SEQ ID NO: 31 - RNA, 87 nt, Artificial Sequence
  Description of Artificial Sequence Synthetic oligonucleotide
agcuucccuggcucuagcagcacagaaauauuggcacagggaagcgagucugccaauauuggcugugcugcuccaggcaggguggug

SEQ ID NO: 32 - RNA, 21 nt, Artificial Sequence
  Description of Artificial Sequence Synthetic oligonucleotide
uagcagcacagaaauauuggc

SEQ ID NO: 33 - RNA, 112 nt, Artificial Sequence
  Description of Artificial Sequence Synthetic polynucleotide
ccaccccgguccugcucccgccccagcagcacacugugguuuguacggcacuguggccacguccaaaccacacugugguguuagagcgagggugggggaggcaccgccgagg

SEQ ID NO: 34 - RNA, 21 nt, Artificial Sequence
  Description of Artificial Sequence Synthetic oligonucleotide
cagcagcacacugugguuugu

SEQ ID NO: 35 - RNA, 22 nt, Artificial Sequence
  Description of Artificial Sequence Synthetic oligonucleotide
ugagguaguagguuguauaguu

SEQ ID NO: 36 - RNA, 80 nt, Artificial Sequence
  Description of Artificial Sequence Synthetic oligonucleotide
ugggaugagguaguagguuguauaguuuuagggucacacccaccacugggagauaacuauacaaucuacugucuuuccua

SEQ ID NO: 37 - RNA, 22 nt, Artificial Sequence
  Description of Artificial Sequence Synthetic oligonucleotide
ugagguaguagauuguauaguu

SEQ ID NO: 38 - RNA, 87 nt, Artificial Sequence
  Description of Artificial Sequence Synthetic oligonucleotide
ucagagugagguaguagauuguauaguugugggguagugauuuuacccuguucaggagauaacuauacaaucuauugccuucccuga

SEQ ID NO: 39 - RNA, 22 nt, Artificial Sequence
  Description of Artificial Sequence Synthetic oligonucleotide
agagguaguagguugcauaguu

SEQ ID NO: 40 - RNA, 87 nt, Artificial Sequence
  Description of Artificial Sequence Synthetic oligonucleotide
ccuaggaagagguaguagguugcauaguuuuagggcagggauuuugcccacaaggagguaacuauacgaccugcugccuuucuuagg

SEQ ID NO: 41 - RNA, 22 nt, Artificial Sequence
  Description of Artificial Sequence Synthetic oligonucleotide
aacccguagauccgaacuugug

SEQ ID NO: 42 - RNA, 80 nt, Artificial Sequence
  Description of Artificial Sequence Synthetic oligonucleotide
ccuguugccacaaacccguagauccgaacuugugguauuaguccgcacaagcuuguaucuauagguaugugucuguuagg

SEQ ID NO: 43 - RNA, 72 nt, Artificial Sequence
  Description of Artificial Sequence Synthetic oligonucleotide
agguugagguaguagguuguauaguuuagaauuacaucaagggagauaacuguacagccuccuagcuuuccu

SEQ ID NO: 44 - RNA, 22 nt, Artificial Sequence
  Description of Artificial Sequence Synthetic oligonucleotide
ucccugagacccuaacuuguga

SEQ ID NO: 45 - RNA, 88 nt, Artificial Sequence
  Description of Artificial Sequence Synthetic oligonucleotide
ugcgcuccucucagucccugagacccuaacuugugauguuuaccguuuaaauccacggguuaggcucuugggagcugcgagucgugcu

SEQ ID NO: 46 - RNA, 74 nt, Artificial Sequence
  Description of Artificial Sequence Synthetic oligonucleotide
gggugagguaguagguuguauaguuuggggcucugcccugcuaugggauaacuauacaaucuacugucuuuccu

SEQ ID NO: 47 - RNA, 22 nt, Artificial Sequence
  Description of Artificial Sequence Synthetic oligonucleotide
ugagguaguagguugugugguu

SEQ ID NO: 48 - RNA, 83 nt, Artificial Sequence
  Description of Artificial Sequence Synthetic oligonucleotide
cggggugagguaguagguugugugguuucagggcagugauguugccccucggaagauaacuauacaaccuacugccuucccug

SEQ ID NO: 49 - RNA, 81 nt, Artificial Sequence
  Description of Artificial Sequence Synthetic oligonucleotide
cccauuggcauaaacccguagauccgaucuuguggugaaguggaccgcacaagcucgcuucuaugggucugugucagugug

SEQ ID NO: 50 - RNA, 22 nt, Artificial Sequence
  Description of Artificial Sequence Synthetic oligonucleotide
aacccguagauccgaucuugug

SEQ ID NO: 51 - RNA, 84 nt, Artificial Sequence
  Description of Artificial Sequence Synthetic oligonucleotide
gcauccggguugagguaguagguuguaugguuuagaguuacacccugggaguuaacuguacaaccuucuagcuuuccuuggagc

SEQ ID NO: 52 - RNA, 22 nt, Artificial Sequence
  Description of Artificial Sequence Synthetic oligonucleotide
ugagguaguagguuguaugguu

SEQ ID NO: 53 - RNA, 89 nt, Artificial Sequence
  Description of Artificial Sequence Synthetic oligonucleotide
accagacuuuuccuagucccugagacccuaacuugugagguauuuuaguaacaucacaagucaggcucuugggaccuaggcggagggga

SEQ ID NO: 54 - RNA, 70 nt, Artificial Sequence
  Description of Artificial Sequence Synthetic oligonucleotide
ggcacccacccguagaaccgaccuugcggggccuucgccgcacacaagcucgugucuguggguccguguc

SEQ ID NO: 55 - RNA, 22 nt, Artificial Sequence
  Description of Artificial Sequence Synthetic oligonucleotide
cacccguagaaccgaccuugcg

SEQ ID NO: 56 - RNA, 79 nt, Artificial Sequence
  Description of Artificial Sequence Synthetic oligonucleotide
cccgggcugagguaggagguuguauaguugaggaggacacccaaggagaucacuauacggccuccuagcuuuccccagg

SEQ ID NO: 57 - RNA, 22 nt, Artificial Sequence
  Description of Artificial Sequence Synthetic oligonucleotide
ugagguaggagguuguauaguu

SEQ ID NO: 58 - RNA, 86 nt, Artificial Sequence
  Description of Artificial Sequence Synthetic oligonucleotide
ugccagucucuaggucccugagacccuuuaaccugugaggacauccagggucacaggugagguucuugggagccuggcgucuggcc

SEQ ID NO: 59 - RNA, 24 nt, Artificial Sequence
  Description of Artificial Sequence Synthetic oligonucleotide
ucccugagacccuuuaaccuguga

SEQ ID NO: 60 - RNA, 22 nt, Artificial Sequence
  Description of Artificial Sequence Synthetic oligonucleotide
acaggugagguucuugggagcc

SEQ ID NO: 61 - RNA, 83 nt, Artificial Sequence
  Description of Artificial Sequence Synthetic oligonucleotide
ugugggaugagguaguagauuguauaguuuuagggucauaccccaucuuggagauaacuauacagucuacugucuuucccacg

SEQ ID NO: 62 - RNA, 119 nt, Artificial Sequence
  Description of Artificial Sequence Synthetic polynucleotide
aggauucugcucaugccagggugagguaguaaguuguauuguugugggguagggauauuaggccccaauuagaagauaacuauacaacuuacuacuuucccugguguguggcauauuca

SEQ ID NO: 63 - RNA, 22 nt, Artificial Sequence
  Description of Artificial Sequence Synthetic oligonucleotide
ugagguaguaaguuguauuguu

SEQ ID NO: 64 - RNA, 84 nt, Artificial Sequence
  Description of Artificial Sequence Synthetic oligonucleotide
aggcugagguaguaguuuguacaguuugagggucuaugauaccacccgguacaggagauaacuguacaggccacugccuugcca

SEQ ID NO: 65 - RNA, 22 nt, Artificial Sequence
  Description of Artificial Sequence Synthetic oligonucleotide
ugagguaguaguuuguacaguu

SEQ ID NO: 66 - RNA, 21 nt, Artificial Sequence
  Description of Artificial Sequence Synthetic oligonucleotide
cuguacaggccacugccuugc

SEQ ID NO: 67 - RNA, 84 nt, Artificial Sequence
  Description of Artificial Sequence Synthetic oligonucleotide
cuggcugagguaguaguuugugcuguuggucggguugugacauugcccgcuguggagauaacugcgcaagcuacugccuugcua

SEQ ID NO: 68 - RNA, 22 nt, Artificial Sequence
  Description of Artificial Sequence Synthetic oligonucleotide
ugagguaguaguuugugcuguu

SEQ ID NO: 69 - DNA, 23 bp, Artificial Sequence
  Description of Artificial Sequence Synthetic primer
tgggcactgtgctaaataaatga

SEQ ID NO: 70 - DNA, 20 bp, Artificial Sequence
  Description of Artificial Sequence Synthetic primer
ataccgcctcttaacccccc

SEQ ID NO: 71 - DNA, 20 bp, Artificial Sequence
  Description of Artificial Sequence Synthetic primer
aatcgttagctcgaagcccc

SEQ ID NO: 72 - DNA, 19 bp, Artificial Sequence
  Description of Artificial Sequence Synthetic primer
cttctctcggcccaagacg

SEQ ID NO: 73 - DNA, 20 bp, Artificial Sequence
  Description of Artificial Sequence Synthetic primer
ctggctctgattggcaagga

SEQ ID NO: 74 - DNA, 22 bp, Artificial Sequence
  Description of Artificial Sequence Synthetic primer
accttagggtagagggagggct

SEQ ID NO: 75 - DNA, 21 bp, Artificial Sequence
  Description of Artificial Sequence Synthetic primer
ggagagactgggagcgagtgt

SEQ ID NO: 76 - DNA, 20 bp, Artificial Sequence
  Description of Artificial Sequence Synthetic primer
caaccttcgaatcccgaaag

SEQ ID NO: 77 - DNA, 18 bp, Artificial Sequence
  Description of Artificial Sequence Synthetic primer
ctccatctgtgagcggcc

SEQ ID NO: 78 - DNA, 27 bp, Artificial Sequence
  Description of Artificial Sequence Synthetic primer
caaaatagtaacgacgagtgaaaagaa

SEQ ID NO: 79 - DNA, 20 bp, Artificial Sequence
  Description of Artificial Sequence Synthetic primer
gctcttgacgtccttgcgag

SEQ ID NO: 80 - DNA, 21 bp, Artificial Sequence
  Description of Artificial Sequence Synthetic primer
aggtgaggaaactgaggcagg

SEQ ID NO: 81 - DNA, 20 bp, Artificial Sequence
  Description of Artificial Sequence Synthetic primer
caccaactgaaaacctgcca

SEQ ID NO: 82 - DNA, 20 bp, Artificial Sequence
  Description of Artificial Sequence Synthetic primer
tgcgcgtgaccagaaaagta

SEQ ID NO: 83 - DNA, 20 bp, Artificial Sequence
  Description of Artificial Sequence Synthetic primer
cctttcactcccagcccaat

SEQ ID NO: 84 - DNA, 20 bp, Artificial Sequence
  Description of Artificial Sequence Synthetic primer
agggagccaacatggagaca

SEQ ID NO: 85 - DNA, 23 bp, Artificial Sequence
  Description of Artificial Sequence Synthetic primer
actccaaagactgtgtttctgcc

SEQ ID NO: 86 - DNA, 23 bp, Artificial Sequence
  Description of Artificial Sequence Synthetic primer
agcaggtgaaaacaagctgaatt

SEQ ID NO: 87 - DNA, 25 bp, Artificial Sequence
  Description of Artificial Sequence Synthetic primer
tgaggtagagtggaaactggagaga

SEQ ID NO: 88 - DNA, 23 bp, Artificial Sequence
  Description of Artificial Sequence Synthetic primer
agtggcatcttaaagcagcacac

SEQ ID NO: 89 - DNA, 26 bp, Artificial Sequence
  Description of Artificial Sequence Synthetic primer
gcacgaatgaatataaaaacaccaga

SEQ ID NO: 90 - DNA, 20 bp, Artificial Sequence
  Description of Artificial Sequence Synthetic primer
agctgccttggcgtcagtaa

SEQ ID NO: 91 - DNA, 20 bp, Artificial Sequence
  Description of Artificial Sequence Synthetic primer
cccaatcaggtgtcggaaag

SEQ ID NO: 92 - DNA, 21 bp, Artificial Sequence
  Description of Artificial Sequence Synthetic primer
gctccctcgcctttagtttga

SEQ ID NO: 93 - DNA, 21 bp, Artificial Sequence
  Description of Artificial Sequence Synthetic primer
ccctcgtcatactatggcacg

SEQ ID NO: 94 - DNA, 21 bp, Artificial Sequence
  Description of Artificial Sequence Synthetic primer
taccatcagcagaggcagtca

SEQ ID NO: 95 - DNA, 18 bp, Artificial Sequence
  Description of Artificial Sequence Synthetic primer
gtcgccccttcccaattc

SEQ ID NO: 96 - DNA, 19 bp, Artificial Sequence
  Description of Artificial Sequence Synthetic primer
tggcctggcaggtactttg

SEQ ID NO: 97 - DNA, 17 bp, Artificial Sequence
  Description of Artificial Sequence Synthetic primer
gacgggacagcggcatc

SEQ ID NO: 98 - DNA, 19 bp, Artificial Sequence
  Description of Artificial Sequence Synthetic primer
ggactcccgcaaaatctcc

SEQ ID NO: 99 - DNA, 25 bp, Artificial Sequence
  Description of Artificial Sequence Synthetic primer
aacattttgttgcttcttggaaatt

SEQ ID NO: 100 - DNA, 19 bp, Artificial Sequence
  Description of Artificial Sequence Synthetic primer
cctccacggtggagatgct

SEQ ID NO: 101 - DNA, 23 bp, Artificial Sequence
  Description of Artificial Sequence Synthetic primer
aaagctgcagtgtccaaattctc

SEQ ID NO: 102 - DNA, 21 bp, Artificial Sequence
  Description of Artificial Sequence Synthetic primer
ggcaggacccgaaataagaag

SEQ ID NO: 103 - DNA, 21 bp, Artificial Sequence
  Description of Artificial Sequence Synthetic primer
gtgccgaggagggatctagaa

SEQ ID NO: 104 - DNA, 26 bp, Artificial Sequence
  Description of Artificial Sequence Synthetic primer
agattgcttcctgagagtagacaaca

SEQ ID NO: 105 - DNA, 21 bp, Artificial Sequence
  Description of Artificial Sequence Synthetic primer
cagaaactgcacacccactcc

SEQ ID NO: 106 - DNA, 22 bp, Artificial Sequence
  Description of Artificial Sequence Synthetic primer
gggctgctgtgtttacaacaac

SEQ ID NO: 107 - DNA, 24 bp, Artificial Sequence
  Description of Artificial Sequence Synthetic primer
caaagagcaagtttaaaagacccc

SEQ ID NO: 108 - DNA, 26 bp, Artificial Sequence
  Description of Artificial Sequence Synthetic primer
acaggttatttgataacccaaggaga

SEQ ID NO: 109 - DNA, 20 bp, Artificial Sequence
  Description of Artificial Sequence Synthetic primer
gtaccagggtctgagcccag

SEQ ID NO: 110 - DNA, 18 bp, Artificial Sequence
  Description of Artificial Sequence Synthetic primer
agcagcagcctcccacag

SEQ ID NO: 111 - DNA, 19 bp, Artificial Sequence
  Description of Artificial Sequence Synthetic primer
ctatggacgccctgtgtgc

SEQ ID NO: 112 - DNA, 16 bp, Artificial Sequence
  Description of Artificial Sequence Synthetic primer
ggctttgggcgggagt

SEQ ID NO: 113 - DNA, 21 bp, Artificial Sequence
  Description of Artificial Sequence Synthetic primer
gcaggacaatggaaggaaacc

SEQ ID NO: 114 - DNA, 19 bp, Artificial Sequence
  Description of Artificial Sequence Synthetic primer
aggccttccgacgactcag

SEQ ID NO: 115 - DNA, 19 bp, Artificial Sequence
  Description of Artificial Sequence Synthetic primer
ccatctggagagcgaggga

SEQ ID NO: 116 - DNA, 19 bp, Artificial Sequence
  Description of Artificial Sequence Synthetic primer
tccgtcttttgcctgcctc

SEQ ID NO: 117 - DNA, 20 bp, Artificial Sequence
  Description of Artificial Sequence Synthetic primer
tccgtcgccattttatttcg

SEQ ID NO: 118 - DNA, 26 bp, Artificial Sequence
  Description of Artificial Sequence Synthetic primer
agaagtttccgatgaacatatgaaga

SEQ ID NO: 119 - DNA, 21 bp, Artificial Sequence
  Description of Artificial Sequence Synthetic primer
gttttcgcggaacaccttagc

SEQ ID NO: 120 - DNA, 21 bp, Artificial Sequence
  Description of Artificial Sequence Synthetic primer
ctgtcgggaagtgaacacacc

SEQ ID NO: 121 - DNA, 20 bp, Artificial Sequence
  Description of Artificial Sequence Synthetic primer
tgcacctattgtgtccctgc

SEQ ID NO: 122 - DNA, 24 bp, Artificial Sequence
  Description of Artificial Sequence Synthetic primer
cacccacttcttaccaagaactcc

SEQ ID NO: 123 - DNA, 27 bp, Artificial Sequence
  Description of Artificial Sequence Synthetic primer
agtttcactgcttcattctaaatcctg

SEQ ID NO: 124 - DNA, 22 bp, Artificial Sequence
  Description of Artificial Sequence Synthetic primer
cagatttgtggctcacttcgtg

SEQ ID NO: 125 - DNA, 20 bp, Artificial Sequence
  Description of Artificial Sequence Synthetic primer
aaaccacccatccagaaggg

SEQ ID NO: 126 - DNA, 27 bp, Artificial Sequence
  Description of Artificial Sequence Synthetic primer
tgagcaataaacacgattaattcgtaa

SEQ ID NO: 127 - DNA, 21 bp, Artificial Sequence
  Description of Artificial Sequence Synthetic primer
catgcgtaaaaatgtcgggaa

SEQ ID NO: 128 - DNA, 19 bp, Artificial Sequence
  Description of Artificial Sequence Synthetic primer
gggaggagtgttcacgggt

SEQ ID NO: 129 - DNA, 19 bp, Artificial Sequence
  Description of Artificial Sequence Synthetic primer
aactctaacccccgctccc

SEQ ID NO: 130 - DNA, 16 bp, Artificial Sequence
  Description of Artificial Sequence Synthetic primer
tcgtgcaattccgccc

SEQ ID NO: 131 - DNA, 20 bp, Artificial Sequence
  Description of Artificial Sequence Synthetic primer
catggcccatcccctaattt

SEQ ID NO: 132 - DNA, 20 bp, Artificial Sequence
  Description of Artificial Sequence Synthetic primer
caaactcacaacctcccggt

SEQ ID NO: 133 - DNA, 19 bp, Artificial Sequence
  Description of Artificial Sequence Synthetic primer
gagtcctaggtccgcccac

SEQ ID NO: 134 - DNA, 22 bp, Artificial Sequence
  Description of Artificial Sequence Synthetic primer
aaaatagcaaagctcccgactg

SEQ ID NO: 135 - DNA, 26 bp, Artificial Sequence
  Description of Artificial Sequence Synthetic primer
tggtctttttcctcgtttatgaagtt

SEQ ID NO: 136 - DNA, 21 bp, Artificial Sequence
  Description of Artificial Sequence Synthetic primer
ttctctcctgtctggtggtcg

SEQ ID NO: 137 - DNA, 20 bp, Artificial Sequence
  Description of Artificial Sequence Synthetic primer
aggaaacccccgaagagttc

SEQ ID NO: 138 - DNA, 23 bp, Artificial Sequence
  Description of Artificial Sequence Synthetic primer
gaatgaacgttgtgaaatccctc

SEQ ID NO: 139 - DNA, 20 bp, Artificial Sequence
  Description of Artificial Sequence Synthetic primer
gcctcagattggttcgcttg

SEQ ID NO: 140 - DNA, 22 bp, Artificial Sequence
  Description of Artificial Sequence Synthetic primer
ccaccatgtggctatgacacag

SEQ ID NO: 141 - DNA, 23 bp, Artificial Sequence
  Description of Artificial Sequence Synthetic primer
cgttggaaagttgtttaccttgc

SEQ ID NO: 142 - DNA, 20 bp, Artificial Sequence
  Description of Artificial Sequence Synthetic primer
ttatggagcaggctgcagtg

SEQ ID NO: 143 - DNA, 28 bp, Artificial Sequence
  Description of Artificial Sequence Synthetic primer
tagttaataaagaaaaaggccacaacat

SEQ ID NO: 144 - DNA, 29 bp, Artificial Sequence
  Description of Artificial Sequence Synthetic primer
aacttaaaaaaaaattcttccatccttct

SEQ ID NO: 145 - DNA, 23 bp, Artificial Sequence
  Description of Artificial Sequence Synthetic primer
tttttcccttttgcattttgaga

SEQ ID NO: 146 - DNA, 25 bp, Artificial Sequence
  Description of Artificial Sequence Synthetic primer
aagtgctaaagctatggttgactgc

SEQ ID NO: 147 - DNA, 28 bp, Artificial Sequence
  Description of Artificial Sequence Synthetic primer
gaaggattgaaaatagctactgtgttca

SEQ ID NO: 148 - DNA, 20 bp, Artificial Sequence
  Description of Artificial Sequence Synthetic primer
ctattggctacactcccggg

SEQ ID NO: 149 - DNA, 21 bp, Artificial Sequence
  Description of Artificial Sequence Synthetic primer
gctctccctcagacacactgg

SEQ ID NO: 150 - DNA, 23 bp, Artificial Sequence
  Description of Artificial Sequence Synthetic primer
acttcaagatcatgctactgggc

SEQ ID NO: 151 - DNA, 22 bp, Artificial Sequence
  Description of Artificial Sequence Synthetic primer
agtgcattaggtaacaagcgca

SEQ ID NO: 152 - DNA, 18 bp, Artificial Sequence
  Description of Artificial Sequence Synthetic primer
tgcgcagaagctgtgctc

SEQ ID NO: 153 - DNA, 15 bp, Artificial Sequence
  Description of Artificial Sequence Synthetic primer
gtgtcccccattccc

SEQ ID NO: 154 - DNA, 20 bp, Artificial Sequence
  Description of Artificial Sequence Synthetic primer
cccacctggtcctctttcct

SEQ ID NO: 155 - DNA, 19 bp, Artificial Sequence
  Description of Artificial Sequence Synthetic primer
cttctcggtgaccacgcag

SEQ ID NO: 156 - DNA, 22 bp, Artificial Sequence
  Description of Artificial Sequence Synthetic primer
aattgtgtagcctccgtaaggg

SEQ ID NO: 157 - DNA, 20 bp, Artificial Sequence
  Description of Artificial Sequence Synthetic primer
gttgctttttcctgtcccca

SEQ ID NO: 158 - DNA, 20 bp, Artificial Sequence
  Description of Artificial Sequence Synthetic primer
ctgatgtcggtgacagtggg

SEQ ID NO: 159 - DNA, 18 bp, Artificial Sequence
  Description of Artificial Sequence Synthetic primer
caccatttgggtgcaggg

SEQ ID NO: 160 - DNA, 20 bp, Artificial Sequence
  Description of Artificial Sequence Synthetic primer
cctgcacgctaaccctctct

SEQ ID NO: 161 - DNA, 25 bp, Artificial Sequence
  Description of Artificial Sequence Synthetic primer
gttaactgaattactgggttggagc

SEQ ID NO: 162 - DNA, 20 bp, Artificial Sequence
  Description of Artificial Sequence Synthetic primer
gctggttctctactgccccc

SEQ ID NO: 163 - DNA, 19 bp, Artificial Sequence
  Description of Artificial Sequence Synthetic primer
caatcagggaggaaaccgg

SEQ ID NO: 164 - DNA, 20 bp, Artificial Sequence
  Description of Artificial Sequence Synthetic primer
ggtggaaggcctgtcaagag

SEQ ID NO: 165 - DNA, 19 bp, Artificial Sequence
  Description of Artificial Sequence Synthetic primer
ggaacccgctgacctagga

SEQ ID NO: 166 - DNA, 19 bp, Artificial Sequence
  Description of Artificial Sequence Synthetic primer
catggccctgtctcccaac

SEQ ID NO: 167 - DNA, 20 bp, Artificial Sequence
  Description of Artificial Sequence Synthetic primer
cgtgactggagaccccagtt

SEQ ID NO: 168 - DNA, 20 bp, Artificial Sequence
  Description of Artificial Sequence Synthetic primer
ttagaggcttcagcaggcca

SEQ ID NO: 169 - DNA, 23 bp, Artificial Sequence
  Description of Artificial Sequence Synthetic primer
ctcttctgggtccttgtagggat

SEQ ID NO: 170 - DNA, 20 bp, Artificial Sequence
  Description of Artificial Sequence Synthetic primer
gtacggagagggcggatatg

SEQ ID NO: 171 - DNA, 22 bp, Artificial Sequence
  Description of Artificial Sequence Synthetic primer
gttagggatatcgaggttggca

SEQ ID NO: 172 - DNA, 18 bp, Artificial Sequence
  Description of Artificial Sequence Synthetic primer
gggtgaacgcctgggtct

SEQ ID NO: 173 - DNA, 20 bp, Artificial Sequence
  Description of Artificial Sequence Synthetic primer
aaattggcatcgggacagag

SEQ ID NO: 174 - DNA, 17 bp, Artificial Sequence
  Description of Artificial Sequence Synthetic primer
cattctgcccacccgct

SEQ ID NO: 175 - DNA, 23 bp, Artificial Sequence
  Description of Artificial Sequence Synthetic primer
agcactatgagcccttctgacat

SEQ ID NO: 176 - DNA, 17 bp, Artificial Sequence
  Description of Artificial Sequence Synthetic primer
accgacagcgtgttgcg

SEQ ID NO: 177 - DNA, 23 bp, Artificial Sequence
  Description of Artificial Sequence Synthetic primer
catggaccaaaatatggcatcat

SEQ ID NO: 178 - DNA, 17 bp, Artificial Sequence
  Description of Artificial Sequence Synthetic primer
acagtggccaatcggca

SEQ ID NO: 179 - DNA, 26 bp, Artificial Sequence
  Description of Artificial Sequence Synthetic primer
gctttaagttgttcaccctcaagtta

SEQ ID NO: 180 - DNA, 24 bp, Artificial Sequence
  Description of Artificial Sequence Synthetic primer
caatgttttccatgttggatcaaa

SEQ ID NO: 181 - DNA, 16 bp, Artificial Sequence
  Description of Artificial Sequence Synthetic primer
cctgcgttggtgcgct

SEQ ID NO: 182 - DNA, 21 bp, Artificial Sequence
  Description of Artificial Sequence Synthetic primer
cgtggcagcactcgtaagact

SEQ ID NO: 183 - DNA, 23 bp, Artificial Sequence
  Description of Artificial Sequence Synthetic primer
cgactggagcacgaggacactga

SEQ ID NO: 184 - DNA, 26 bp, Artificial Sequence
  Description of Artificial Sequence Synthetic primer
ggacactgacatggactgaaggagta

SEQ ID NO: 185 - DNA, 23 bp, Artificial Sequence
  Description of Artificial Sequence Synthetic primer
agagctgctgctgctgatactgc

SEQ ID NO: 186 - DNA, 23 bp, Artificial Sequence
  Description of Artificial Sequence Synthetic primer
tggggacaacagatttgcattga

SEQ ID NO: 187 - DNA, 25 bp, Artificial Sequence
  Description of Artificial Sequence Synthetic primer
gtcaaccctctgcatacccatctcc

SEQ ID NO: 188 - DNA, 25 bp, Artificial Sequence
  Description of Artificial Sequence Synthetic primer
ataaaaagttttgggagccctgagc

SEQ ID NO: 189 - DNA, 25 bp, Artificial Sequence
  Description of Artificial Sequence Synthetic primer
gctgtcaacgatacgctacgtaacg

SEQ ID NO: 190 - DNA, 23 bp, Artificial Sequence
  Description of Artificial Sequence Synthetic primer
cgctacgtaacggcatgacagtg

SEQ ID NO: 191 - DNA, 25 bp, Artificial Sequence
  Description of Artificial Sequence Synthetic primer
ctcctactgaactgtcacggcagac

SEQ ID NO: 192 - DNA, 24 bp, Artificial Sequence
  Description of Artificial Sequence Synthetic primer
gtatggattcattgccaggagctg

SEQ ID NO: 193 - DNA, 26 bp, Artificial Sequence
  Description of Artificial Sequence Synthetic primer
tccaagaactcacacattcaggcaaa

SEQ ID NO: 194 - DNA, 25 bp, Artificial Sequence
  Description of Artificial Sequence Synthetic primer
gtctgccgtgacagttcagtaggag

SEQ ID NO: 195 - DNA, 25 bp, Artificial Sequence
  Description of Artificial Sequence Synthetic primer
ttcagctgggatatctctgtcatcg

SEQ ID NO: 196 - DNA, 23 bp, Artificial Sequence
  Description of Artificial Sequence Synthetic primer
gggcttgaggacctggagagagt

SEQ ID NO: 197 - DNA, 24 bp, Artificial Sequence
  Description of Artificial Sequence Synthetic primer
gctgaggatacacatcggcttttc

SEQ ID NO: 198 - DNA, 25 bp, Artificial Sequence
  Description of Artificial Sequence Synthetic primer
ctcctcgttgtgctactgtctcctg

SEQ ID NO: 199 - DNA, 29 bp, Artificial Sequence
  Description of Artificial Sequence Synthetic primer
accgctcgagggcacagaatggacttcag

SEQ ID NO: 200 - DNA, 30 bp, Artificial Sequence
  Description of Artificial Sequence Synthetic primer
accgctcgagcatgccctgctcagatcttt

SEQ ID NO: 201 - DNA, 32 bp, Artificial Sequence
  Description of Artificial Sequence Synthetic primer
ataccgctcgagcggcagggtgtctgtctagt

SEQ ID NO: 202 - DNA, 29 bp, Artificial Sequence
  Description of Artificial Sequence Synthetic primer
accgctcgaggcatgctctcccatcaata

SEQ ID NO: 203 - DNA, 38 bp, Artificial Sequence
  Description of Artificial Sequence Synthetic primer
ataccgctcgaggatcctgaatgctgtgcctgttcttt

SEQ ID NO: 204 - DNA, 31 bp, Artificial Sequence
  Description of Artificial Sequence Synthetic primer
ataccgctcgagcctcctgcatcctttcttt

SEQ ID NO: 205 - DNA, 32 bp, Artificial Sequence
  Description of Artificial Sequence Synthetic primer
ataccgctcgagagagatccacccacatcagc

SEQ ID NO: 206 - DNA, 31 bp, Artificial Sequence
  Description of Artificial Sequence Synthetic primer
ataccgctcgaggagtgggtgtgcagtttct

SEQ ID NO: 207 - DNA, 32 bp, Artificial Sequence
  Description of Artificial Sequence Synthetic primer
ataccgctcgagtcccctgagctgagttccta

SEQ ID NO: 208 - DNA, 31 bp, Artificial Sequence
  Description of Artificial Sequence Synthetic primer
ataccgctcgaggagcggattcagataacca

SEQ ID NO: 209 - DNA, 32 bp, Artificial Sequence
  Description of Artificial Sequence Synthetic primer
ataccgctcgagatggcttttccccttcagat

SEQ ID NO: 210 - DNA, 32 bp, Artificial Sequence
  Description of Artificial Sequence Synthetic primer
ataccgctcgagctctccaacttgcccaaaac

SEQ ID NO: 211 - DNA, 32 bp, Artificial Sequence
  Description of Artificial Sequence Synthetic primer
ataccgctcgagcaggcttccaatggatcagt

SEQ ID NO: 212 - DNA, 32 bp, Artificial Sequence
  Description of Artificial Sequence Synthetic primer
ataccgctcgagaccacatgcaattcaggtca

SEQ ID NO: 213 - DNA, 31 bp, Artificial Sequence
  Description of Artificial Sequence Synthetic primer
ataccgctcgagatccctgccagctagacaa

SEQ ID NO: 214 - DNA, 31 bp, Artificial Sequence
  Description of Artificial Sequence Synthetic primer
ataccgctcgagcctgtgcctttttccttcc

SEQ ID NO: 215 - DNA, 31 bp, Artificial Sequence
  Description of Artificial Sequence Synthetic primer
ataccgctcgagcctgagactctgccttctg

SEQ ID NO: 216 - DNA, 31 bp, Artificial Sequence
  Description of Artificial Sequence Synthetic primer
ataccgctcgagagcgcaccagaggatatgt

SEQ ID NO: 217 - DNA, 32 bp, Artificial Sequence
  Description of Artificial Sequence Synthetic primer
ataccgctcgagatttccctctcagcttcgtg

SEQ ID NO: 218 - DNA, 32 bp, Artificial Sequence
  Description of Artificial Sequence Synthetic primer
ataccgctcgagcaggacctgaccttggacat

SEQ ID NO: 219 - DNA, 888 bp, Artificial Sequence
  Description of Artificial Sequence Synthetic polynucleotide
ctcgagggcacagaatggacttcagttaagtttttgatgtagaaatgttttattattctacttaaaatctccttaaaaataattatgcatattacatcaatgttataatgtttaaacatagatttttttacatgcattctttttttcctgaaagaaaatattttttatattctttaggcgcgaatgtgtgtttaaaaaaaataaaaccttggagtaaagtagcagcacataatggtttgtggattttgaaaaggtgcaggccatattgtgctgcctcaaaaatacaaggatctgatcttctgaagaaaatatatttctttttattcatagctcttatgatagcaatgtcagcagtgccttagcagcacgtaaatattggcgttaagattctaaaattatctccagtattaactgtgctgctgaagtaaggttgaccatactctacagttgtgttttaatgtatattaatgttactaatgtgttttcagttttattgatagtcttttcagtattattgataatcttgttatttttagtatgattctgtaaaaatgaattaatactaatttttcagatgtatcatctcttaaaatactgtaattgcaatttaataattgtattgaatgccatcaagtttttttaaaaagcttatgcagcattagaggaatttattttaatgcacatttatattcaacatagacattaattcagatttttacttgggataaaacaaattctagttttccctttgttttgaaattacttttaaaatatgtctttacagataaatataaaatatattaagcattttgaacagagcttagaagacaatatttagtactgtttctgaatatttctttatatctgaaggggaaaagccatctcgag

SEQ ID NO: 220 - DNA, 220 bp, Artificial Sequence
  Description of Artificial Sequence Synthetic polynucleotide
ctcgagcctcgggaagccaagttgggctttaaagtgcagggcctgctgatgttgagtgctttttgttctaaggtgcatctagtgcagatagtgaagtagattagcatctactgccctaagtgctccttctggcataagaagttatgtattcatccaataattcaagccaagcaagtatataggtgttttaatagtttttgtttgcagtcctctgctcgag

SEQ ID NO: 221 - DNA, 283 bp, Artificial Sequence
  Description of Artificial Sequence Synthetic polynucleotide
ctcgagcatgccctgctcagatctttcccattttccctccctttcccttaggagcctgttcctctcacgccctcacctggctgagccgcagtagttcttcagtggcaagctttatgtcctgacccagctaaagctgccagttgaagaactgttgccctctgcccctggcttcgaggaggaggaggagctgctttccccatcatctggaaggtgacagaaatgggctgggaaggtccgaacagcagggtggatgatacgttttgggcaagttggagagctcgag

SEQ ID NO: 222 - DNA, 325 bp, Artificial Sequence
  Description of Artificial Sequence Synthetic polynucleotide
ctcgagcggcagggtgtctgtctagtctatggtcattgaggggaaaaagtcacttctccctggtgcaattcattacctaatcatgacctggacagactgtcctgtcggagccaaggacagaaagctcccatagaggctgtggctggattcaagtaatccaggataggctgtttccatctgtgaggcctattcttgattacttgtttctggaggcagctgatggtccgccgccggaaacagagatggctcctgggacatggtgtgtgcgcttcttcctgagccaggttgaggttgggaccactgatccattggaagcctgctcgag

SEQ ID NO: 223 - DNA, 1150 bp, Artificial Sequence
  Description of Artificial Sequence Synthetic polynucleotide
ctcgaggcatgctctcccatcaataacaaattcagtgacatcagtttatgaatatatgaaatttgccaaagctctgtttagaccactgagtaactcacagctaggtttcaacttttcctttctaggttgtcttgggtttattgtaagagagcattatgaagaaaaaaatagatcataaagcttcttcaggaagctggtttcatatggtggtttagatttaaatagtgattgtctagcaccatttgaaatcagtgttcttgggggagaccagctgcgctgcactaccaacagcaaaagaagtgaatgggacagctctgaagtatttgaaagcaacagcaggatggctgtgagaacctgcctcacatgtagctgaccccttcctcacccctgccaacagtggtggcatatatcacaaatggcagtcaggtctctgcactggcggatccaactgtgatcgaaagttttccaaaaataagttgtgtctgtattgaacatgaacagactttcttcttgtcattattctctaacaatactgcataacaattatttgcatacatttgcattgcattaagtattctaagtaatctagagacgatttaaagtatacgggaggatgtgtgtaggttgtatgcaaatactacaccattttctatcagagacttgagcatctgtggattttggtatccaaggggctttctggaaccaatccctcaaggataccaagggatgaatgtaattgtacaggatatcgcattgttggaattttatacttctttgtggaataaacctatagcacttaatagatagtacagactcattccattgtgcctgggttaaagagcccaatgtatgctggatttagtaagatttgggccctcccaaccctcacgaccttctgtgaccccttagaggatgactgatttcttttggtgttcagagtcaatataattttctagcaccatctgaaatcggttataatgattggggaagagcaccatgatgctgactgctgagaggaaatgtattggtgaccgttggggccatggacaagaactaagaaaacaaatgcaaagcaataatgcaaaggtgatttttcttcttccagtttctaagttgaatttcactgacctgaattgcatgtggtctcgag

SEQ ID NO: 224 - DNA, 346 bp, Artificial Sequence
  Description of Artificial Sequence Synthetic polynucleotide
ctcgaggatcctgaatgctgtgcctgttctttttttcaacagagtcttacgtaaagaaccgtacaaacttagtaaagagtttaagtcctgctttaaaccaagtttcagttcatgtaaacatcctacactcagctgtaatacatggattggctgggaggtggatgtttacttcagctgacttggaatgtcaaccaattaacattgataaaagatttggcaagaatagtatacagaggcttgaatttttaatgtaattaatgtaattaaaggtttgttggaaatgtgagaccattttgttctcccagagaaaaagtgtgttaattgtctagctggcagggatctcgag

SEQ ID NO: 225 - DNA, 385 bp, Artificial Sequence
  Description of Artificial Sequence Synthetic polynucleotide
ctcgagcctcctgcatcctttctttcctccccacatttccttcttatcaacaggtgctggggagaggcaggacaggcctgtcccccgagtcccctccggatgccgtggaccggccagctgtgagtgtttctttggcagtgtcttagctggttgttgtgagcaatagtaaggaagcaatcagcaagtatactgccctagaagtgctgcacgttgtggggcccaagagggaagatgaagcgagagatgcccagaccagtgggagacgccaggacttcggaagctcttctgcgccacggtgggtggtgagggcggctgggaaagtgagctccagggccccaggagcagcctgctcgtgggtgcggaaggaaaaaggcacaggctcgag

SEQ ID NO: 226 - DNA, 397 bp, Artificial Sequence
  Description of Artificial Sequence Synthetic polynucleotide
ctcgagagagatccacccacatcagccttccagactgctggcctggtctcctccagatgtttataactcatgagtgccaggactagacctggtactaggaagcagctgcattggatttaccaggcttttcactcttgtattttacagggctgggacaggcctggactgcaaggaggggtctttgcaccatctctgaaaagccgatgtgtatcctcagctttgagaactgaattccatgggttgtgtcagtgtcagacctgtgaaattcagttcttcagctgggatatctctgtcatcgtgggcttgaggacctggagagagtagatcctgaagaactttttcagtctgctgaagagcttggaagactggagacagaaggcagagtctcaggctcgag

SEQ ID NO: 227 - DNA, 413 bp, Artificial Sequence
  Description of Artificial Sequence Synthetic polynucleotide
ctcgaggagtgggtgtgcagtttctgcgactcagggtggcgtccccccaacctgtccctgccccttcctgccctctttgatgcggccccacttcctctggcaggaacccccgccctccctggacctgggtataaggcagggactgggcccacggggaggcagcgtccccgaggcagcagcggcagcggcggctcctctccccatggccctgtctcccaacccttgtaccagtgctgggctcagaccctggtacaggcctgggggacagggacctggggaccccggcaccggcaggccccaaggggtgaggtgagcgggcattgggacctcccctccctgtactcccatctctgctgcggcttttatgcgtctctccccttcgggtcccacatatcctctggtgcgctctcgag

SEQ ID NO: 228 - DNA, 749 bp, Artificial Sequence
  Description of Artificial Sequence Synthetic polynucleotide
ctcgagtcccctgagctgagttcctacagagggaagatggtccaatcttactacactgtgagctcatccccatggtccgtcgccttccagttgcctgctcagcccgtccctggttcctcccaaacgtttttgggggccatgtttgccttttaaggcttctctatccccccgctcctggaggtggtgctggggtcttcccagcactgctatgtgctctcttcctttcaacccaccccggtcctgctcccgccccagcagcacactgtggtttgtacggcactgtggccacgtccaaaccacactgtggtgttagagcgagggtgggggaggcaccgccgaggcttggccctgggaggccatcctggagaagtgacacaaaaaacatctggggccttgtgacaaacttcttgccaggtgggcaaggagagggtggggtatgtaagcacccctctaaaatctccagggcagtttcaagaatactgatggccagagaccctgggagtaagttctgcctcaagagaacaaagtggagtctttgttgcccacacccagcttccctggctctagcagcacagaaatattggcacagggaagcgagtctgccaatattggctgtgctgctccaggcagggtggtgaaaactaccgaggaggggctgagcccccatgggccgaggagagaagagggaacaggcctctcctgctaataatgttaagcagacagcacgaagctgagagggaaatctcgag

SEQ ID NO: 229 - DNA, 1051 bp, Artificial Sequence
  Description of Artificial Sequence Synthetic polynucleotide
ctcgaggagcggattcagataaccaagcatttaaaatactattaatgaaatacaggaaatgaaaccacagcatagattatgcatgtagccaaaatgttcagttaaacttcattttcaacgtaagtgaatgaaaatggtctaatactatttttcttatcactcacacaggaaaccaggattaccgaggaggaaaaaaagccttcctgtggtgctcaactgtgattccttttcaccattcaccctggatgttctcttcactgtgggatgaggtagtaggttgtatagttttagggtcacacccaccactgggagataactatacaatctactgtctttcctaacgtgatagaaaagtctgcatccaggcggtctgatagaaagtcagttaactaattgtacaatatttaagattaacttgtcttaaagagatgtagtgcagcatttgtttatggcctggaaataaattaatttagagataaagtctgtagcaagtacactggatgggggtggggaaaccttttgcttcttgtcttatttctctgtgtcagaataaatgtatttttttattttgatttatgctgataattttatgttgaaattttctttcgaaagagattgtactttccattccagaagaaaacattgctctatcagagtgaggtagtagattgtatagttgtggggtagtgattttaccctgttcaggagataactatacaatctattgccttccctgaggagtagacttgctgcattattttctttttatttagatgatattaaaactcagaagaattaattttgacattttgtatttacagtttatcagttaattttctctgttcaagtagtacagtaggcacagattaacatttaaatttttcacatatggtatatttcagaaatttgaagttaagcaaaaattttaatgagtagagaaagtaagtagccttcaggaaatcttcatagaggaccaggcccttttggaattgtgaataggtttattgccttacatcctggtacacatgtccaaggtcaggtcctgctcgag

SEQ ID NO: 230 - DNA, 40898 bp, Homo sapiens
agttttttggaatgaacgttgtgaaatccctcctttataatggcaggttttcagttggtggttttatcagaatttctcaagatcaaaacgaaaccttctctttaaaaaggaaagaaagtactatcgatacagaaagcaaaagtatttccagtctcctactgaactgtcacggcagacctctctgtatctatatttagagctgtatgtccatatatttgcctgaatgtgtgagttcttggaagtatggattcattgccaggagctggtgatttcctaagcagaggtcgctaactacaagaaatgttacactcggtgagtaggtggtactttctgaagattattggtaacccagtgttggtcgtcttgagtcatggatttaaatgacggctagcaagctcttaaacaccttctgctttatatgatacagagtatggaactgcaggacgcacagacaactgtgaagggtttttaagaatatggattcattgaataatcagtgaatcttgtgaatatactagacctttggaggaggaaagggtgttttacagaaggaagtggtttgtgggggcttttaaaggtacagttatcgcttttctatatttattttcagcatcattcagtcctcctatatgaaagaaagaaatggtctcgatagagaaaaagttatgctgactctttaaatgaagaaacttgatcctcatgttactcttaactggacttttttcctcctaaatattcataagggaaactatcccatgaaggcaactaactttctggcaatctctatgatgtgtctaattcagaacccggtgggctctccatgcattattggcagcacttttatccaggggcacagacagggaatagggttactggaaacagggagtgtagctcaaggaattgttctctctctggagtggataagttgtttgacctactgactcatttcataggaattaggaaaacaacctagtgtggtttttattgttagtcctggaggattccaaaatctgtgtgcctgcaaataagatgagctggggaaagggagcaccagcatttggtgtttctacttgcctcttttcctgatggaatccaatcctctgcttccaggacaagtcctgcgcttggggatcctctgtacgcccgttcactggtgagtatgtatgacagcaatttctgatttcactagttcttgcttctaacacctaaatggtgctttgtctagggaaacgctttaaagagcattttcccccagtacgtttataatacatttcatatttacctccagaaaatactgtggatgtgataataattataaaagacaacccacagacaacaatgaaagttaagaattaagatatattctgatagatttttgcagcatattgacataaaggcagttttggttttgacatatgtacactaaaatgatatctgtaatgctattaaaactcaagtgtacaaggcaccagtatattctggcaaaaataatataatgatcaacagttaatgaaagacttgtgctttgagcttctaacaaaatgttaatatgtagagaatcttgtgattcatgatgctttggttctcaaccagggttatcaaataagctaaaagtatcagaagaggacaaagaaagtacgtaaaacatggataaaagcagaaaccgagaaaagatagctttgatatttaaagtgttagattatggcctctatataatgtcatatatgccagatccccccccaaaatcacaccctcaaattatgatcttcattattcaaaagatgtttggcgtgttggtggtacagtggtgagcgtagttgccttccagaagatggttggtgtataattactatagaatttcttgctctcttgggaggtggcttagtagaaggttgagtcaggcaagctggggctgtttattcagtgtgctgtgctgtgctgtgctgtgctgtgctgtgctgtgctgtgctgtgctgtgtctgaatgcctgggagtttgcgttgttaacctttccagggtgctagatccctttgagattatgatggaagccatgaatccttttcccacacctgtatgcaaacatgtgcctgcccatttacagacccaaagaaggacatccatgtaccccacgttaagaaggctgccctggactaagccctcactgtgactctgctgttcttaagcttcccaggaaacactgttatgtaaaaagacgagccatccaggtcaaaaagaagtcattgtggttgctaggtgggggtgcaaagccaaagacaaagggtggggaaaatacatgactaataataaataaagagcatggttaataccagagtctccaaactgatagcccagaatccctcaacatgtagccacgagtatttgtaaagtacgttgtgaattcaggtagagggccgaagactcaatattaagcagttgggcagagttttcatacatatatttgtctctgccttttgcacaatataaaactatgaaatctcttttcatttttctattgtgcacgctaccacattaggtaggaatcaaaatcatctcaactccagaagtataattcataacaattataacaaagcagaaaaatgattctacattagcagaagaaatgtagtaggaatagatttaaaatagcatggttgagtacaaaagaaacatcgttttttaaaaaatggcagcctttatcactcatcagctaaacttaaagtcaatgctgtgaggctgttgcgggaaaaaaatagtcatgattcttgggtgtattaaaaggagtatgacatagaagggtaagaaaacaagacgtagtgtgtttgtcagactcatcattgtgtcctgtttgagtcacacttaaaaaaagaatttggacaattgtgaaaagctgatacacaagcaacagaaattacacgcccttccaggaaaagataaggcaactgaggattttattccacacaaggctggaggattttttgctaatggcctttaagtagtaacagtctggtttatccttatattcctagggccttccgctctgcctggtgcacagtacttgagatgatcgttgtgtttagtgataatgagtaaggttcctgtgagagacttagcaggagtaagtaggttcaaataaaaccagcgatagtgtcatgacagacggtatataatacatgaatttgctgggtgattaatacataaggatgactgtctcacttgtgaggatgatataatacttcagcagctaactgggggaagctacaaaatcttggtttgatacctctttcaggatgaaagcagataagtctgctggggtggtggaaacattcattcattcattctacaaaccctgagggcctacttggctaggtgctcacggtctggtagaggagacaaagatacaaaaacagagtcacagtgtgtaatgagtgccatgggagcataaaaacagcatgcgcagggcttgtgacactcaaccgccaccccgcgcagcttccaatggaggcaaagtcacaattgaccccaatagacaggcttgacagagttagttggatgggaggcaggtggggcagcctgtccagaggcgcagacctgtgtgtgtggggaaggggtgatgggagatgcagactcggcagtgcttgttaacagaggtgtgaatgccgcgccttgcttgcctgaagatttccacccaggatcctgacaaaagtcagatgtgcacttcagaaaggccaggcgggctgcagtgaagagaatgactcgagggggtgcgggtgtgggacaggccatcgaggcaaggaggccaaatggaaagctttggtcaggacacaggtggtgatggattcgggggagaaatgggggagggtgacgcaggcagagtggccagggttatcaagtggcatcattcaccacaaaatgcagtccaagaggaagcccagctgtgagggggggacaggtggggcccaggtgtgaggggggataggtggcattcagacatgtcagtacacagagtcaatggcagagacagagccagtgggcagttgcaccatgtggataaggacttgagtgagacctgtctccaaagcaggggatcactcttctaattttttatatggtgtttttcattctattctgactcatttgtaataaaacctcctttgttttttatcatatatatttcaggtgtcaacatgctggtttgatataacatgtacaaagcaaaataataacttacactccagctgtgagtgtaccatctcacagttacctttttgtgcatgtgtgttaagcccacctaaaatgcacttagaaatattcagtatcttcactgtggtcatgctgtactgcagatctctagatgtattcgtctgacatcattccagctttataccctttgacccacatctccccatccccccaaccacctctctctgtttctatggactgtatttgactattttagattccacatattacctctaatcctcaaagctctattagggttttgtgtatttccttccagcattttcctacatacacacaatttgcagaatcagcataattctgtataggtactttatttatttatttattttgagatggagtttcgctcttgttgcccaagctggagtgcagtggcacaatctcggctcaatgcaacctctgcctcctgggttcaagcaattctcctgcctcagcctcccaattagcttggattacaggcacgcgccaccacgcccaggtagttgtgtatttttagtacagacggggtttcaccctattggtcaggctggtctcgaactcctgacctcaggtgatccacccacctcggcctcccaaggtgctgggattacaggtgtgagccaccacgcccagcctgtatagacactttcctctttttttttttttttttttgagacggagccttgctctgtcacccaggctggagtgcagtggcgcagtctcggctcactgcaaactccacctcccgggttcatgccattctcctgcctcagcctcccaagtagctaagactacaggtgcccgccaccacgcccggctaattttttgtatttttagtagagacggggttttaccatgttagccaggatggtctcgatctcctgacctcgtgatctgcccgtctcggcctcccaaagtgctgggattacaggcttgagccaccatgcccggcctgtatagacactttctatcccacttttaaaacaggcatgttctccctatgctttaaaatgcctttaaaatatttctaatggtgctaattctctaatttgggagtgtctaatttgcttccagtttttaaaaactattttttattatagtaattaaccttttaaatacatgaatcttgggcatatttttaatgatttccttaggagagattccttacagtgaatttcttgagtaaaaggaggttaactgtaagattcttgatataccttgctaaattgttttctagaaagcttgaaccaagttctgcacattttagcaaagtatgaaatgtccttctctccatatcttcatcagcattgagtgttctctcttttttatctttagacaaaaaggttacattctttttggttcatctactcagaagctatttaatgaatgttcactccatgtcaggcatgtggcatgttttcatctctaccagtaacgctgaactttcttcttgtgtgcatcagcctgttgttttcttttgtaaatgttctgttcgtgtccattatcaacttttctactagggtgtgactgtttctatgatatatttataacgatgtgtgtgtgtgtgtgtgtgtgtatacgatatttggggtaaatacttttcccagcttctttgacttttaattttgcttatactttattggaaatacagaacctttattttattattattattatactttaagttctgggatacatgggcagaacgtgcaggtttgttacataggtataaacgtgccatggtggtttgctgcacccatcaaaccaccatctacattaggtatttctcctaatgctatccctcccatagtccgccacccaccaacaggccctggtgtgtgatgttctgctccctgtgtccatgtgttctcatgagaacctttatattttatgtagtggagtaagttattttatctttttttttttcctgtgtaacttctgttacctcaggcttaggaattccttcctacccagaagttagaaaatattcatctatttttttttaaagtttcatttttaacattaaactcaccaaattagaagtaagatgtggccaggcacagtggctcacacctgtaatctcagtgctttgcgggggctaaggtggaaaaatcacttgaggctaggagttcaagacaagcctgggtaacacagtgagaccccacctctaccaaaaaatttaaaaattaggcaggcatggtagcatgtgtctgtagtcctagctgcttgggaggctgaggtgggaggatcacttcagcccagaagcttgaggctgtagttagctattatcatgctattgcattccagcctaggcaacagagcaagaaccctgtgtctttaaaaaaaaaaaaaaaaaaagtgtgaaacaaagatccaaatggactttgtttcacaaagtaaatcccccatcatgttttgagtcatgtatctttccctatttaatttgtggtgcttcctttattttatatttcaagactttttgctattatatccagcggtaggagcccatgaacttgttggatattggtatgcacagaagatgcggtaatacttttttccaatattttattatgaaaaatacactggctagattctataattaatattctgtcactttaccatctattcacgtaataattcttaaacttctcaaatcattatttgaatttcaaagtggactatcaaagacattttctctaaccctatttttctctaatttgcagttttgatttccaggaagcctttagcaatttatattgttagtacagtaagagagtaaacaacacactaaaataccaagagaaactactttccaaaacatgttttagtcgtttaactttatgaatatggctagcaagggggcttcctgggggacagtgctggggaaagagcaggccctaggaatgggctttgaaagaagacttttgccaccgtcaggtcacatttgcaagaggccatcgtcgtggtctgccagggaacccttagcagaaaggctgctttgcccaggatctggcttgccgccccagaccagccctaccgctgtctggcattgaatgtccctggtccgcagtgagtggccagttgcatagaccttagatcaaggagcttagtcacctctttccagcctaaatcatgagcaccctaaattcccatgggttgttgagacctaatagcatcctggcttagagatgggattgcagctgggtctgcacaggaaactggcctactgtgtctgcctcttggtgcagcatacaaccagccacatggaaaaacccagcctgcaataaaagacttgctttggtcaccacctgacattagctgtggtttaatctgtgtaatgtttttcttcccccccatctaagcctcaatttcctatccataaaatatgttggttttggccaggcgtggtggctcacgcctgtaatcccaacactttgggaggccaagatggcctgatcgcctgaggtcagaagttcaagaccagcctgggcaacatggcaagaccccccccctctactaaaaatacaaaaaactagctgggcgtcatggcacatgcctgtaatccccgctacttgggaggctggggcaggaggcaggacaatcgcttgaacccgacaggcagaggttgcagtgagccgagatcgtgccactgcaactctagactgggccatgagcgagactctttctcaaaaaaaaaaaaaagttggttttattagattacagagaatactttctctatccaaaatctgtgattttaatctagaacactgaatgtaggtcagtatccaccccattttcagaaatctgggaagatctttttttgtttttcagcttctcagaataaatactttctaggatgttacaaacatggatgaagttcaccagaacagatccagggttaaccttttaaagtcattagatatggctccagtaaaaggcatgagaaggcacccgtgagaccctgcagaggaagcctcactcctgggcagccttacggctgacgagctaccttactgagcatattcctgcctctacaccagagactcactctgtggtccggtgtcacctcgattctaaattccctgcttcctggtgaatgatgctatcacactttagaaacctggccaataaatgctttgaaatttaaggatagctatcctgaaaaaatttaatataacctaaattgatagtctaatgacatcagtattcagaagaggcattctatttcagcaagtggttttcagaaaaaaaaaaaaaataaataacctgagtttttcccctgccactctcccctcttgcttttatgctgcaaaaagagcttgtggcttgcctgacacaccgtgttgttctctgccttcgaagatctagctccctcagcctccccacgttgtccacctctccaataccattgctccactgggaagctggcccggaggtgctctaccacgttcatctctcgcagcccagggctgtctttaacgttacagagacacagatatcaaagtgttgcaatttatttggctttcattaatttgccggaagcaagggagggtagatacaaagtactcctctaacctgttattttccttgctgttaaaatgtaaagaagactgatgtaagaatctctgccttccaccagtcataaccatggtatgcacagagcttatgaatcatcttctaaattcacagctagttttaaaatttagcttccaggctagatctgcaggaaatgattgggagatcgtaagtgctatggagtgaatatgaagttacttaccagatcccataaagttagctcagggaatcctgaggaaggtgtcactcagaaagctgctttcaaagctgagtcagacctgcatggtgtttattacctccattgtgttaccatgccaaacatataaatagcttcaaagtgagttcagattaaaacacagaaaaatctttggatatattcacaggccagtgggggtcatgtaacaagtcagcaaggaaaataaaagatatttgaggtacagcctcaactttttaagatggatcttgaataaaaaggtctaatgaaatattttatggtattgctctctgggatttaaaaaaacaaagcaaaacaaaaaaacgagggcactggaacaatgttttagatagatcacgggtgttttccagtatgcatttttagtgcacttacttaactgagagccctctcatatgaaactggaatgtcaccaccatgtgataaagcgtagactagctgtgacctaatacgtactgatcatgactaacgcagaagtccaggtcactgcggttgtatttcctgtggcctcaatgttctggggttagggttaacagggataattcatttctgtgttcaagtctctaattcttccatcatgtaaactgtaagtgggagcagactggattcagcattgctacaagagaaaaacaacaaataggaacaccaccaacaccatgtgttgggagccccatgtgccaggcactgtgccaaatactcgatattgattatctcttttaatacacaatcctgtgtggcagatcctactatgagacccattttacagatagacaactgaggctgggggaagttggaaatggaggagccaggattcaagtcacagcagtctggctcctgcgcccaagcttttaatttgtatcctattcacttttcataaataaatgactcgtgttggaggtgtttgtatccttggggccactcagttttattcagccagctttccttggttgtggttttaatgtgttagggaagaatgaatgtattccccacagctgggcatctttgtgctggatcacctggccacagctcatgaatagtgcagattatcacaccccacccccatgactccagcaaatgtgctgtgggtataggagagtatgttcaaaaaaggcaattaacaaagcaactgtcccaactaccaagaaaaacaaatgtggcataacccatgatagaaccctcttttttccttattagacatgaatagaagctcattaatgccgtccagtaagttcaagccgcataacttacatacactgtcaggacccagaaagaaacctgttgtcttgtgatgttattgttgcttggttgacctcatcatgtcttgacagggctcaagacacccttcatgatctggtcttacctctcaggactccccccatccttaccattgtttgttgatctctggtgcagccaaatgaagcccatcatgcttgtcctctgcctggaagctcttccttccctcttcctggccaatggctactgtcccttcagagcacctgttcagatgaaacctccaccaggcagcctctgctgactcctaagggctgggctaggtgccccttgggtgcttccacagccctttctgcttacccagttgtacatgtgtcatactgtgttctgtaattcctctttttgtccatcttccctggcttgcattgaccccttaaaagccatgattagggccgggcgccgtggctcacgcctgtaatctcagcactttgggaggccgaggcaagtggatcacaaggtcagatcgagaccatcctggctaacgtggtgaaaccccatctactaaaaatactaaaattagctgggtgtggtggcacacgcctgtagtcccagctcctcgggaggctggggcaggagaattgcttgaacccgggaggcggaggttgcagtgagcagagattgcgccactgcactccaacctgggcaacagagcgagactccgtctcaaaataaaacaaaacaaaatcatgatgccatgatcagtgtttacttttggataaacacccggacacatgatagagaataaatgtttgagagagagaaatgacctactgggtaacaccaaaaactaatgtataccatgtctagtgtttacagtttacaagatgctttcacacacataacttcatttgatcctcccggcaactctgcaaaatagatttctgcagccctgttgtactgaccaggaaacatcagcctgccctgatggcagggatgtggttagcaggtggaaagccaggactcaggctcaggtcttcgaacccagattttatgccctatgtaccgccatgtggatattctcaaggcaactctctgaaatatttccagaatctttcacaaaagtcctgtgtcgtccttgtcctggccaactagactcagagggaggactgatggattttcatctccctcttccccttcgtcttttcttctcagcatcttcctaccctccccaccaccgcgcttctccaccaaaagtctgatgctggcatgtaatggaccctttccaagtttcaaggtgttgacagtaaatcttttgtatttagaccgggccggctgctttccttttcttgtgggtagaactctggaaaagtggaggggggtgggtggggcttcaggtaaagtacgtcagcaaattggataagtcggatcttcgttgtgtgagtgggtaagagatgaactggggggaggggtgtgtgtttgggcagtgagcaagccggcaagagaatgtgagtgggcggccagttggttggcaagtgagaggctgtggagaggttgctgctgggacgggctgtgcaattaaacagttgagcagatcttaatagcagactggggcggtgctctccgcaggcctgcggggagcagggagcaggggaagcaagcgagctggaagcatcttcgggcacagagctagatacgaatttcagaagcacataacctgaggaatagaatgcaaccagaggagggagaaatagagattccaggcagcgtttcctaaacttaaaaagttttgaatacccagttttgccttgttctggggacctctggcaatgttgttcaagggaggaatttttaatttcatagaaaagtgaattttaccaccacaagagaacatgagaccaaggtcataaatccctttgtttggagtgtccgttctgaactccttgtatacttttatttgcaggaagaggctataccagcaagtattatggttttactgggtggggcggcaaggccaggaagagatggagatgctgttaaaggtccttgctccagaacattcagagatcagaggaactcaagaggtcaaagtaggctgatgtcattgttcatccatttattcagcaaaactttcttgaacacctactgtgtgccaggcactggttggcagtgaggtgaagacgatagtgatgaaaacaaaggcatggtctctgtcatcatggaacctaagtaaacacataccaaaaaaaatggcccaagaaaaatcatgccctgaggggaaggcactcagatgaaaaggaccttcttcccagtggtcccctgaggaggtgatttccaaggtctcagctaagggatgagcaggaatgtgtgggtgcagggggtaggggtgagagctctcggcatagagagagcagcacatttcaaaggccctggagtggggaaggaggtggcaggtgcagctgtgtggtggctacttccacttctggagtgcagtagcatgatctcgactcactgcaacctctgcctcccgggttcaagcaattctcctgccacagtttcccaagtagctgagattataggtgcaagccaccacgcctcgctgattattacatttttagtagagacggggttttaccatgttggccaggctggtctcgaactctgtctcctggaacctcttgatgagcccctgtgaggttccccagcccagggcatccccccaggtattcaggagagacccgtccccaagagactgcctgctacttcctcttcctatgaatgtcactgtgaggggaataaataaggatagaccagaaaagcaaattgtaagaaatttgaaaccagttgtcagttttcaggtgcccaggagcatgacaatatgcccgagggaccgtaacaggacttgacatggagctgggtctaaagcagatgacctggtggctgcagcgtgttccacacaggcgagcactgtgaggccaaaggactggtgttgagcagaatgaaaaagcacagtgttggttaatcctgaaaagtgaagcctgcaagaaatgaacttcgaccttggagtgggggtgggacaggggctagaaggaagagaggctcggaagtgtgttgtgtaaacgtggttctgttttgtctaatcttttactacctgtagggacaggtggatgcagtggagctgctggcaggaaaggtggagttttttcaaggtcacgagttaagcaatagagtagaatatctttgcagctgcaagaggaggtgcgagcagcttgttcacaaggtatattttgcaggagtttccccttacaagatcttatttgcttgagagtagcgttaaggatttctgtgccaacagttagctggatctctggggttgaatcgttatcaccaatctatagttaaaataagcattaaaaaaaaaaaggagtctactttcaaagttttcccgaagtgccagtctgaagcagctacctttaaaagctgttcttgaataataggttgctatatcctagtctgtaatcttggactaatgtctgaggaaggaaaaaaaaaaaaagaatattctctctttgtccgattaagaagtggaggtagacagcgattcgacatgttgtatgggcgaggctgtgctgagcttgcgtctgggacaatgaccagacagatgaaaggagaagtgagagatatttttagcacatcaggactgctttgaaacattgattttttttccatctcctcccaccccccataattccagcaggaagattcagtggcaggcactttaattcagtgtaaaaacatcttctatattacacactaaaatattcatgaggaaagggagttgctgattttaaggcgtagctttaaaacccacgtctgtcggcttgatggtcagtgtcccgccacacagtctggaagtgtgacgtggccaaggaggacccccagaaggatcctgtgtcttccgtggggactactcctttcctcagaggagcgggttcacaggaaactggagcagaaggatgcctttagccccactcctgttggtcctcagtcccttctcctttgatcaggtggttcaagcacgccttgaagttccggtgttcaaacaacgtgacctctgcaattacgttctaatacttgtcggtgcccaacttaaaccactagcaatgttagttaaaaacatcagggaggccgggcgcgtggctcatgcctgtagtcccagcactttgggaggcctaggcgggcagatcacttgaggtcaagagttcgagaccagcctggccaacatggtgaaactctgtctctactgaaaatacaaaaaaagttagccaggtgtggtggcgggtgcctgtaatcccagctacttgggaggctgaggctgaggcaggagaatcacttgaacccgggaggcagaggttgcagtgagctgagatcgcgccactgcagtctagcctgggtgacagagacaaaacacaaaacaaaacaaaacacctcaggaggctgtaaatcaagcacaactgaagcaaagataaaaacgggcagtctcagaatcacctttgaaggtggaaggaagggcgtttgtgtgtgacaagaagagtgtacgttcatacaagggtttttactcctcctctgaaaagtcctgcttggtggctggccagtttgtcatgcttggctgcccaagcaagtggtggcagtcggagcacaggagaaagccgcagggggtgaggaagaagtgctggagagggcgctccacctttcggctgccctgacctgtctccagccccagggcatgtggttctgggccgggcactggccccctctctgtggactgtaccaggcagcgggtagtgcacaagctctgaggtcaggccacctgggcctaaactccagctctgctccttgctagcagtgtgactctgggcacgttcaagttacttcatgctttgtgcctcagtttcctcatctgtgaaatggggatggtaacaggaccctcatcccaggactgctgtgtggattgaagtgcctgcaacgggatttgtcttttctgggatcgcggtggatgctctattccaggtgagggagtctcttgctttctcctacctcttgcagccgctttgcctctgagtggcaaatagcaagtggctgaaaatcacaatgagcccgtcagagaatggggatgggccacaaggccacagtcggatgggttctccatgccctcgactatgcagcggggtcagggcatcccttcctcctggggtgcccacaccattcccttggaagtttagacagcagcccttgcctcgccagacgcagcaacccctcccagctacacgtggaatctgtggcccagcctccctcagatgacccgcattgcctgacagagaccaggggccctgaggggtggctgtcgctggcctgggacagggtccctgcaggttttcagggtcctcagcagcattcgccagcgtcgggtcagactgagtcatggggcccgagctctccacgagttggttctccagttggttctcgttgggggtaatgcccttcatcgaaccgcttcatccccagcctgtattgtctccgtctcttcgcagttgtcagagggctcggccgccaccctgaaacagcccagagctctctgaagagcacgagcttttacgcttctctccccaaatcaactgactttttcattgcttctgtggaggctttctgtgggagaaaagcgtgtttatggggcaagctcagagagctcctgtccccgtggtggaatttttttagtggctctgcccacgtggtggtgagcgccggtgaggatgggacgcattttctgctaaaatggctgtgcgaggctgccacctcgtggccagcacaaggcagtgagtgtgtcccttgctggagcttctgggtgaaataaactcattttaaaggtcttcagcaaggacctcactccctcactctctcagtgtgcatatcattccagctccagggcttaatgcgtctgttgtggcatctctcaaatctgtactttgctatctctttgactacagagtcactaccttccgataagcccttctgctgtctccagaccgatccttctaaagcgtagatgtcattgtctttctctcattaaagctctttcatggctcctcattttccaggttccagctcatgtctcaggaggccgctaagcccactcctctgccccagcctgtgtctgcgcacgtctcttctcccttcctcctggtttcctctggctgatacctgctcacctgaaagtcatctcaggtgccacctcctccagggattcctcgcattagattagcttccttcttccccattcctagctgacttccttcattatgcctcagatcctttttttggaagaaaaagaaagaaatggaagacagactggccagcagggggagagagagggagggagggcatatcagagaattcaatatgctgcgttttaaccagatacttttcttcctctcccacaaggcatcagagtagagactgcctttcatctctctcccccttgtgtaatagcacagtgtctggcagataactgttgcttgctaattattagaaggaagaaagggagggagaataaaagccagcatcaccatgtgatcttcctaagtgcgacagatgtgagaaaagaactaccttaagctccaggggccctagaggaacggcgctgcgtgctagtgcagatggtagcaaacgctctcacctggcttggcaacacttcttcacctcttctttctaccaccacagcgactgtctccctccagcccatcctactgccaaattcatcttctattaagcatagcttcaatcacatcatttctttatcaaaacacttctacagaggagttccaaatgctttagccgggccgtccagggcctccacaggatgaccccagcccatctagttttctaccactctttatgtgtttcctaagtaccattcattattttaggacttggtatttgctgatgaatttacttacttacttgagagtgtggcaaaagcacatcctaaactttcagttaaaaggagtttgattgattgacctgctctgagtttaaggcacgtgctggtcactgtgggagccattaaagaaggtattgttctgtggccgccaggaacagatagcccagctggggacaagacaaaaaagcagggggcattaaggaacagacagcagaaaggaacatgtcacaagtgcacaattaatcgccaaatatatggctcaagaaggggtggtgatcaaaccagataatgtctgtgaaagtgcctctctcctgtcaaactcccaattccgagcagtcctacagtcggccccttccacagggcgtagccctgtcattccacctcaaacgtcgtacacactcggcctgtcctacaaagcccttcgtgataaggcctgcttctccttcccaccttgtgggacccccgaccctggaaccccgagctgcagagctggagagaatgcactctgttgtttggtatacgtgttccttgaaacattcactcctccccattttccttcggtgtttaacctgttcttcctttaaaattcggcttttcctttaaaatccagcctggagctccccaatgaagacttctgtggcgctgcaggcaaaactcaccacctgcagagtctctgctctcccttgtcaaatacgcgtttgtgacatacattggaaggcatcgggcacatttctgtcatctgtgtgactgtggacctctgccgcaggggtcatgtcctttccctttacagtgcttgatgaggtacacgtcttgtcaatgtttgtcacggactgaaacttaacacattagagaggacagcaaagtcagttgactctaaattgttaaggcacaagctctgtgagttcagaagagacatgtgccagaatcggtccagggaggttcttagaacctaccagttctaagtaggagttcttagaacctaccagttctaagtaggaattcttagaactggaaggtcaaagctttgtacatgtgaaatagatcaaacaataaaaagaattccatgtagaatagctcagtgtctgtggctcctaatggctcccccattctctgcaccagccagcgggaggagaaagcctgcagagtgggggcagcggagacagaacatcttaaccccagcagaggctttatctaaggaagtagagcgaatgccctgaaggtgggttggacttaggtggtggaaaggtttcaaatgcactctgttatcttgcaggtagtggctgccatttattactttagaaaaccatggcgacttcatcaaagtggtgttttagaaagatgatccagggccgtcttttccctttgagagtgggaggtgccctgtggccctaccttcattttctgtgatggtattcccagtggtttcctccctgtggctttgcggcttagtggtgaaccaagaggcttaggatgcaggtgcaaagcccctctactctttaatgcctcacttcaaggtgcaggatctttccctctgtaaacaaaggctttccttctggcactgcccttgacctgctcagagctgttctctgatcctttgggtctctctgccctcgtgactggcagtgacctgctaggcagcaacccccacgcagcaggtggtcctccagagctgagtcaccaccccgggaaccgtgccctggtgaggccatccaaggtagaccggcacagggcgggtgtgagtggcctctccacctggcccctcggtctgggagggctctttcagcttgacagagagagcaaaggaaactcggatttttggcaaagcagaaaaacattttacctgtgatcacagttatgggccttggtcagtcatttgatttcagcacacccaaattgtccatttacagataccttgagacatggaaagggaggaaattttttaatgatgttttaagattctgtttgacctcatgaattaagagcggtgagaagtaatagaacacggagagatacctaagccattgattgatattgtaggcaataaacaagtgagattcagattatttttaaaaatttttttcatgggcactgatggatgtttaaaaaattatgacgtgttctgttcatgacgcgttctgttcatttccagagaagagaaaaatgtggttgcttttgaaaggaggttttttgttttgttttgttttgtttttaaacgtctagcttattcattacaccagcgaatagcaatgacctcagtctaaaaagcaagttaaccatcatctctctccttgcctgggaaaagaagcctttatctaagtagccaaaagatgggcaaggctgaccacccagtcatttcggctgatcaggattttccaaaaaacaaaaagctctctgttttcacaactgagtgacaactcttaggaaatactaccagcttactcagtttgagtaaggccatggagaagtctggtgacagtcagttgcctggatgcacatgttacacatggctaacgatgctgtcaccactgggagctttcagaattggagaaaataacaccgtgtctcttgctttctgaacatggaatttttaactttttttttttttttttctggagacagagtctcgctctggcgcccaggctggagtgcagtggcatgatctcagctcactacaacctccacctcccgggttcaaacaattctcctgcctcagcctcccaagtagctgggattacaggtgcccaccaccacgcctggctaatttctgtatttttagtagagatggggttttgccatgttggccaagccggtgttgaacccctggcctcaggtgatccacccacctcagcctcccaaagtgctgggattacaggcatgagccaccccatgcccagctggaattttttaacttaaaaaaaaaaagtttcagtgtttgtccttacccattatgtgcccagttagttctataccagtgaaagttccccccaaaagaaaaagaaaaagaaaaaaaagactgtcattttcttgagcagctttcttgagctctgtgtgtgtatgcctatgtctggggcatacacaaggactctggcaggcccactaaactctaccacgtagttgccagatatgctgtaaactgtcacctggaagttgggatttactcttttatttttctgtttattttgcatgagtactgtatgtataaaaaagtagccacatatggatggtcaaagggtttcctagaaatatggatagtcagagggcttcctagaaatatggatggtcaaaggtatcctagtttttcaggtaaactccactagaaaccagaaccatatcccaattccatagcctttattatttcagatcaatagaagcagaatcaaaatgcatttggagcttagaggtcaattgatttggaaaccactccggtgcaaagaatctctagattcattgattttggagtattgtttaggggaaaaggactttaaaggctgggcggggaagctttattccttttttttttttttttttttttttttttttcagaagacatttatgagtttattgatttggcacatttccagaggacaattcagtgacatataccaacatgtacaacagattcatcttttgacccaactttttcactctttttttttttatttttttttatttaatgttttttttttattatactctaagttttagggtacatgtgcacattgtgcaggttagttacatatgtatacatgtgccatgctggtgcgctgcacccactaacgtgtcatctagcattaggtatatctcccaatgctatccctcccccctcccccgaccccaccacagtccccagagtgtgatattccccttcctgtgtccatgtgatctcattgttcaattcccacctatgagtgagaatatgcggtgtttggttttttgttcttgcgatagtttgctgagaatgatggtttccaatttcatccatgtccctacaaaggacatgaactcatcattttttatggctgcatagtattccatggtgtatatgtgccacattttcttaactttataacctgtgtggggcaggagaattaacctgcttttggattagaggctgcaaaaaagccagttctggtgaccacactaaaaagattaatgagcacttagcaccaaattaaatacccccctgcccccgcctttatgaaagtggagttcatagtacaaatgtgacagcgttaaacattgaagccattcgaaggaacctgggaagtactcaggcaccgatgagttaaaaacacagagacatccagtaagtgagttcccgctgggcacgggtctgtttatcttgcctttctgaagtcagagccaacattcttaatggttttgaatgattcagaatgtttcttttttggcagggccagtttccacgcatgggcttatgactaaaaccccttaggtgaggtccaaagcagaaaccagcctcacacttaatgtcgtatttcagaatcatcttattttagagcaaaaggaaaactattaaatatagcttgcttatttcagacatgaagggacggaaccagggaggctttggtcagccagaggtcacaggactggagagagaggccagcccgaggcagacctctgggttctgggcccagccctccctcctctgcaccacacagctcttgtccagacctcggaaggaggttccagcgtggcctcagcctcccgctggaagaggcagcttgccctttgagatcgtttagaaaacgacttgggtcagaaaggagaacttcatttttacaaaatgaaacaaatcagttatttgccatagacagctttttcaaaatatcctttgccagtgtgtcgtgaatgtattagctctgggacagaaacaagaataataacaattctatctactatatgccaaaagaagagacttgatatacttcttttttttttttttggatgagaatgaacttatgccacactagttcttaataaaacaaactgaatccttaccacaggagaaagaattgagcaatgttctaagaacaggctgtcctctttggggcactgagtgaatgtaatatttagaagatgcccttgaagctcacaagtgcctcttgggatggagtaaagggggagaaacggcgagagaactcggacagagaagctgtccatgggaggagaaggccttgggaagagccatcgactcacccccttcctgcttgattgaggactttgggaatcacagatgccataggagagaacccatttcttcaaagccaggaggtgaaccggcccacccctgctgtgcccaccacactacagccagtaagggttctcctctggacctgagccactgtccccaagggccagaaaccagccatgtgtggcgtccccagaaaaggacatgagcaggtactcagccccgccacacacatttgtctaaggagagaaagcaagtcagcgaaatgggatctgcaccaagatctctgcacccagggagctgctgtggagtaaatcctatttttccaggtggtgtttaaacagctgtcattgatcactttgctttttttaattgattatttcgaaacgtgttttcccccaaaggatgttagtatgtcataaatattcagttaagcaactccaggatgatcttttagtaggaggagctgtcttttggtcctagtaacactaagtgccattttttccctcttctaagcccatggaaacaacgtggaggaaaaatgattatatatttagcttctagcatgattttagagataaatgacagccacctcccagacctcattcccaacacctgtattaatttttattttatttttatttttttgagacagagtctcgctccatcacccaggctggagtgcagtgctgtgatctcttagctcgcctaatttttgtatttttagtagagacggggttccgccatgttggccaggctgctctcgaactcctgacctcaagtgatccacctgtctcgtcttcccaaagtgctaggattacatgcatgagccactgcgccccaccttgtctttatttttaaagtaggcaatttattttaattttttgtgtgtgtatgacagggtctcgttctgtcactggagtgcagtggcaccatcaaggctcactgcagcctcaacctcctgggctcaaacgatcctcctgcctcagcctcccaagtagctgggactacactcaccaccacacccagctacttttaaacttttttgtagagacatgcagtcttgctgtgttgcccaggctggtctcgaactcatggcctcaagcgatcctccagccttggcctcccaaagtgctgcgattacaggtgtgagccaccatgcccagcccagtttatttttaaaagagctactggctgggcgtggtggctcacgcctgtaatcctaacactctgggaggcccaggcgagtggatcacctgagatcaggagtttaagaccagcctggccaacatagtgaaatcccatctctactcaaaatacaaaaattagctgggtgtagtggggcatacccatagtctagctacttgggaggctgaggcaggagaatcacttgaacctgggaggcggaggttgcagtgagcctagatcgcaccactgcctccggcctgggcaacagagcaagactctgtctcaaaaaaaaaaaaaaaaaaaaaaagctactgaagagaggagtttcgctgtgttaagaccactgtgcgtccataatggccttccctcagatctctcttccagggagctagggctgtctacacatgctccgtagaaacaaatgcacattttctacacgggagtgaattggagagtcgtggtggtgatagtttaactgaaatctaccttggattttttttcttctctgctttatatttgtttaaaaaagcatgatccagcaaatgcatcaaattaaagctgtgggtacagttctcctaacagaaaatcacacagtaagctgggacaatggaacagaaagcagtgggtattgttttcttcttaagatttttatattcacattggatgagaatgtctcctaaccctccccttgctgttgttcttggaatgggtaccttattcatggcctccttacatagagggagcagattcaggacagaggctctgcatgctcctgggtccctcctaagcgagcactcccctccactgctcctcctgttcggcatgctagattttctgtgcacttgcagtcatacggaaactggcttccttcttagctgtgcgtgtgaaagtcaccccagtgtttcacctgcagtgtgagtgtaatttggggtcccatgacatgatcatcttccttttgatttctaaagctagggtcagggaaggggactcgttttgagccaaagggcattttaagggcatcatttcatatgttcactgtctgcagccaacactgcactctagacaaatgggtacctttatgtcccatttattaagacatcctgcaaaaggggctcctggcccctgtgggaagcttgaaaggaacagaccatatcctgtttctttttgctcatacctctgtggtgagatcttattctgaccccatagtctggtgccatacctgtgggtccctgtatctcctagaattccccctatgctagtgtttagcccgtgtaatgacagttatctgtccacagggctgtatcccccgccagcttgtaaactccatgaagaccggccctgcgtctcactcactcctgtccacagcagtgcctcccacatgttaggtcttaacaaacatctgtttcttctactcttcataaaatccacgcttcttctcacccccccgtcatccctgatcccagctaactttgaccctgtgtgcgggggcgggggatctacatgtgggctccacccccgcacgcctgctgccctgtcatctctgatccctggcggtgggatgtctaccatggctctgcccaatcccccactgtcttcacccctgccacatctggcatgattttaccgtccccttcctagagttctccctttctcccctgcccaggctgcacccctggagagcccagctcaggcccctggcccctatgaagcctctcctgaataatccatcagcctgccctgagctcatccagtctccaatgacgattgtgctctccaccagcaggacacaccggagcactttctctgtccctgagaattggactccattccacagttaggtcctgggtcacatacagacatttcacatcccctcctcagtattccccagcagcgtgcagcacagtgcgggcttctaggaggcagtcggtggatatttacccatgattttaagaggtctgtcttgaccaggaatgttgagaagtgacctgtgtctctgcagcattgctgctagttagtccctcgtggaagaagtggcatgtccctcctgtgtctggctcacggggaccggcgcctcctgtgtttgcccccgccatgccctgtggtttcctttgacaccctctgtcattgtctatttttatgttttaaacttcagtgggatttacctctcgtttgctcccatatcctcctggtaaattgtaaacttggtgaatcaatgatgagtaatcagtaggcagaaatggttggaatttcctggtcccagtttcagatcatatctgtctttccccagactcctaacttcctgttgggacatcccttggtctggcctttccctccagagaagcccccagacccacccgcagccttgatttactgatgcctaatcttattttagtctttcacaccagtttttaacaactcgactgtatagatcttctggaaattttacccaaaaatgttagatgtgccatcccctcacaggggaaagtggtggggtggtgggggttaatgtgttgaaaatctatgtatcttgtattttatttgagtcagctgtgttttcacaacaactctgagagttaacatcatttctactttataaattaaaaaaacaaaacaaaaacaaaaaactaaagccccaaaaggattgaatagcttgccaaggctgtgtaacaagtgaaatttgaatccgggtctgtatgatcccaaagcttaagctgcccgtctttctattcccagttcaaacgtgccttgaaatgggagctcacttgatagtacttggcttttaaaaggctacgtacatgtaagtaccttcactcaaatatgctggggctcagtaggagaagcaaagagaaggaaaaggtattgattgatcgatttgattctaagcagagattgacagagataagcttctaagacctttcaggccccagagtgtgactgttgagcacctaagaagacctcacttgacccgtgacttcccgccagagccttgggaagcccatgagtgcagcgtgctcgccggctgggctggaacttgctgctcttctctgcagtggcaggctggcttctgcccctagactggaactgccaagggctgctatagaagggacaaacttccagtgctcactgaggagccaggggtatcctgggaagctgaaaatgcatttgtcaagctcaggtcttcacaattgagaaaccccatcactaacccgtacggttttcttgaccaaaactttcagtgctctgtttttcccatctgtaaataatgttcatacatctaaatcctaggaggttgcagggacagtggcttagaaagtcaccacattctctaaaggagtgacagagaggatgaaagaagcatgctctataattaggttttattacatgctttttaaggttaattgaaggacttcccagcagcttccctagccactttttcaaatactgaaagcttccagaaagcagcagacaggtgttcctcacaatctgacacagacttcagttactgttgaaaagtgaaagagggctgggcgcggtggctcacacctataatcccaacactttgggaggccgaggtgggcggatcacctgaggtcaggaggtcaagaccagcgtggccaacatggcaaaaccccatctctactaaaaatacaaaaattagctgggtgtagtggcacacgcctaaaactccagctacttgggaggctgaggcatgagaaaaaatcgctcgaacccaggaggcggaggttacaatgagtcaagatcatgccactgcactccagcctgggtgacagagtgagactatctcacaaaaaaaaaaaaaaaaagaaaaaaatgaaagagctcctgtctgacgttggttttctatacccatttttataatcagacaattttggaaacatgctattatgatagtgtttaaatattccagaattctgtgtgttcggtagcttcacgttggtttgattgttgttttaccccactgcctcttccttcccatctgtacttctcagcttgattttttagatgtattttctgttgtaggacagtatcaaatggctttttctgaccatttattttactggccgagctcctcttggattttaatctcaactctcaagaatcttcccaacataatctgttcctgttgtgatatcatctttcttgagagcttcccagtcactgaagagcatgttcagtagcacctggcttaaagagtccacctgcgaatcacaaatcattgtcttcaccattcagctccgacccacacatctgggcaacatcatccccactggccagtaagcacggggaatgctctgtggcctcagctgtgttctggttcctcggtggagctgtaatcattaacttagaaattgcaattggtggtaacttaacagagctttcttcatcagactgctgagtcatgcagcctcttgctgggtaaaatgtgtcttgagatgtaatcccctataaggacacccccttggagccactgtgaaggcactcccagcttccctagcaggtcgcatcagggggacgactgaaggatttacttgaacaatcagttttcagaaatgaagaccctacgaccttggcttcctctcttgtgatgcagctgaaagatgcatttcgtctgtaaagttagaccaggtaggagagaatgtgaggtggtttccattcttctcagttgccctccacctgattctgaagccgtcacagaccgtccttgcagaccggcgctcccctccacaaaccttctgcagcctgtactgctacagcacgaggctgcagaatcagttgtgaaaatagaagccttctaccgtctcctgtgcgggatccttgcctgcctttcttctgtgatgggatctataggcttagactgtgttttatttcctaagacaccttgctgagcaaaccttgctgctctgcctcgtgtcagcggtttccttagcaacggccacacctcacgttttctcccgcacagagcaccagcatgagggggcatgtgttgctcactgtacaggaactttcccaacctgtgtgtcttaagcaggaaagagaaggtgctttctatttcttgtttttaaaaatgcatctgttggtgacactggtggagaaacatgtttacgagaaaacaactgactttttcttctgctgttactccaggagatttcctaggagctcatactgtcatttaaattccaggtgcaggtcttctgaacaaatgcctcctgaggatttctgtgcctggaaaatatctgttaacagactgcttttacacctacgtaagtagaggcttctgcttataagtcaacctcaagggagttttattttgtccctggatgattcctcagtacatttttctttttcttttttgaggtagggttttgctctgttgcccaggctggagtacagtggtgtgatcacaactcactgcggcctcaacctcccaggctcaagcgatcctcccacctcagcctcccaagtagctgggaccacaggcgtgtaccacccacgcctggctagttttttaaaatttcttttgtagaggcagagtctccctgtgttgccctggtttcaaacttgggccaagtgattctctgccccagcctctcaaagtgctgggattacaggtgtgagccaccgtgcctgacaagcctgatatgtttttttaaatgatcattagtatttgatctctactctttttctctcaaccacccaactcccggtttacggtcaggaagaatctataacatgaactcctgcttaaaaacaagggtatagatcccatattcaggtcttagactgggaagaagagaaggcagtcaaaaatattaaatatatgaatttcagtaaattcagttggggatgccatttcagctttgcagtcagttactccagaatatgtcaatgtatggcaaagcatgtgacagcaggatctctgcaccctctccatttaccagctgtggtggatctcttagccacatctctgtttataatgtgcttattgaaaactaatgtattacttttattatcatagctaccatttactgaatatttacattgtgctaacactggttcagatggcttcatcatatttacctgatttaatcctcacaacaaccttatgtgctaggtccagttattaccatctttttttttttttgagacggagtctcactctgtcgccaggctggagtgcagtggcgcgatttcgtctcactgctgcaacctccgcctcccaggttcaagtgattctcctgcctcagcctcccaggtagctgggactacaggcacatgccaccacgcccagctaatttttgtatttttatagagacgagacttcaccatgttggccaggatgatctcaatctcttgacctcgtgatccgcccatcacagcctccaaaagtgctgggattacaggcgtgagccaccgcacccggcctattaccctcattttacagatgaggagactgaggcacagagagttaaagtgatttgtgcagggttgtgtgaggagctggtggcagagcttggcattctggctctggagtctgcatttggccatctcaccgtacagtgtcatagaaacaggaaactcgagaagataaactgccaggctgtattttggagtcagtattttaacactgtcaccatctcaagcatgatctgtgggactcatcactgtgctctttgacttgaacttctttaaggttagtgggaaaatgaagctttagagaagatgggactggcctaaggtcacactgcatgtcagaagtgtggtggagggtccaggtcttctgaccattcagtgcaagccaaattgtgctaatttccaatttttctccctttctcttggtgagctatttggtgctacttctactcctcatgaccaaagctggctttcatggtttactcatgtacttactgatgaaaccctgagacttggggccagtgactgttcagaggtccatgtgaagatgcgctgaatggtataaaggacagagcagtaaagagtgctggctgaatgcctgggctggcaatcaggcatgttggagtttgagtcccagtctgccactccagctctgtgaccaagaggaagtgaagtcatgtaccctttctaagtctctagcccctcttctgaaagacgaagatgccaccaccaaccatgttagtggttgtgaatgccaagaaagtccatgtgaagcacatagcatagtgtctgccatgcgttaagtgttccaaaaatggaagctatgactatttcacagagtacacttcccatcacttttcagttcattttggccttctccctcaaaagcactccagtgttctgatccagctgataaaacaccaaacattcacatagtgctttaaaagtgacccactgtttttacagactgcattacatatgaccctaacaaccactgcaatatagaaattcgcatctccattctccagatcaggataccgaagctcagaaaaattaaatgatttgcccaggatcacatagttagtaagcagcccagccagatcctgtgttaacttgttcatccgcttcctataagaaaacatccattcaaggcacatggggcaacaaagaaggacttagagctgtgtggcccctcgtcagggcagtaccagttgcacccatagtccggcctgaatactcagcttaggactattgtgtccaatgtcccaaacattttgaagctacatcttaaagaagtgtcctgctcagatgcagttctccttgtgaaatccgcaggccccaaggaaagtgactttaatccagtttgctcatgcaagaacagactggcatttcacatccaggaaaacaatggatttgtactgttcagcttttgccatgcaaggagttcctggtgtggtttattaatttacatctcaggctccagcttcactatcaatcagatttggaagaaaaaagttacaaagaaaggcagcttgctgagaaacagcggaagcatgacggtacagactgggttttgaaatggagacacattctcatggcttgtgggctgagagacactgagaaaggattttgttcttgaaagatgattgttttaatgctggagatggagagttttgacaattttaagtgcatgttctgtgtgtaagaaagagtcattacgttttttttttctttcagatgttgccatgccttaaaattgctgatgattaaaatagaacatcctgagttacagaaattcagccctagtgtatcctggcctaaaaatacagaacaatcaagttgattgttggaaatgagaggctaggcagggttggaaacatgctaatgtttactgagtgaaatctttccttctcagtagagttgcccttgcagctgaaagtcactgaaagactcaacaaaataagcagatcccacttgtctcactctgttctcctatgcatgccctggaaagagaatgaatgagccctttttaatttatcaactggttttcttccatctctttactagtgagccatgggggttttttgccgttaactgggtagccagtctcttcatggagacttattttcaggaaactagcctcttgctttatgtgaaaacaaggacccaactcaagatgtcttatcacagtgtgttctgttgcgtaacagttgcatctgtagtccagcctcaatactcaggttgggactgttttgtcctgatgtctcgaacatttcaaagctacgttcttaaagaaatgtcctgctcagatagagctctccttgtgaaatcctcaaaccccaggggaagtgaccttaagccagtttgctggcttttcatgtcacagaaaacaatcgactgccactactcagctgttgccacacaggagtacctggcgtggtcactacttgccaactcagtaccccactttctccccaaggacataaaagcaatgtgggaagatataccaaatgaccttgtcacactgggctaatatgaagagcccacagatgcaaaataatgtatcatacctttattcttctgattttttttttttttttttttttcggagacagtctcactcttttgcccaggctggaatccagtggcgcgatcttggctcactgcaacctctgccacccgggttcaggcaattctcctgcctcagcctcccgcccagctaatttttgtattattaattagtagagagagggtttcaccttcttggccaggctagtcttgaactcctgacctcgtgatacacctgccttggcctcccaaagtgctgggattacaggcgtgagccaccgtgcctggccttttttttttttttttttttttttttttgagatggagtctggctgtattgcccaggctggagtgcagtggcatgatcttggctcactgcaacctctgcctcccaggttcaagtgattctcctgcctcagcctcctgagtagctgggattgcaggcatcccatacccggctaatttttttgaaacagaggcttgctctgtcacccaggctggagtgcaatggcacgatctcagctcagtgcaacctccgcctactgggctcaagcaactctcctgcctcggcctcagcctcactacaggcatgtgccaccacgcctggctagtttggttttttttttttgtatttttttagtagatatggggtttcaccatgttggccaggttggtctcaaacttctgacctcaagtgatctgcctgcctcagcctcccaaaatgctgggattacaggtatgagccaccacgcttagccttctgctgaatttttgagaagcatacataaagacaatgtaaaaaacttagcatatttgtttggccctgctaagggcatggttgaatatatagtgtgtgtgatgtatttggtgtatgagatatatatgatcttatcagacataagtaaaagaaattcattcttcaggaaaaattttccccataactgaagtgtcaatcatttgaaaaaacttctaggcagtaggcaaacactgtgactagttatttatcaaaaataagcagactttagagttttgtaactgatgagaataatctggctgcttcttttgtacttcagtcactgatactgcgaattagagtagcatatacagaaggatgcagggggtccctaaaggaatcacagaattagtgaaaccattatttgagagttgcagtgattcttctgtcctggcacttttcccagttttggtgcttaaatttactatccacttgaaatgtgtcactgcctgtcacttcacaaagtatgtgcctttcactgcagtcaacaagctgtttacatctttgatgaaatgattgccacgcatctccatagccctggtgttctattcccttattcacagagattttttttaaaaaccagcctagcagcaataggtatgtgctgtggatgcttctccatgatgataaaggatgagtttgtgctgggatctccttatgaaaagataactggccaagcccatgaggaacaagaatgttatgctaatcagacccaaggtaaacagcctgcagtgcacctcctgactgattagcactgaacccacttggaatcatcaaccacaggcacgtggtaattggatgttcttctaatacacatagtgagattgtaaaatgaggatgatatatgaggaaagtcagacggagaaactaaagttcagttcttaaatacaagccaagggaacagactaagtgaatggtacagccaggattggagcctggttttcacaaggtggtatgtcatgataaaacaagccaaagaaaaggttgggtagtgttttgaggctaaaagagaccaaagagtcttagtgaatcaaccttgatgcgatctgggtttgaaaaacctataaaaaaccttttgtttggagaaagtgggagaagttggatgtaggctggatatttgtgggtaccaaggaattgtttgttttcttaggtatggtaagattttgtggttatgtagaagagtgtcttcattttatagaacgacctcaaagtagttaggagcagtgtatgtatgggtggttatcaatttactttcaaacaatgcagcaaaaaatatgtatctatattacatataattaaagcaaaacatgataaaaaggaatagttattgaatttagatagtatggatttaggaattcattgtattgtcattccaatttaatgtataaaattttttataacagtttataaaaacagtgcacacccctttaattaggtggcctgggtacatgaagtatctttttggatgcctagaacacaaaaagggacgacaacaggccacctaataagatgataccaagaactataccatgacggcgaaaaagaactgctgcaaaaaataatttgccaaggagaactcagagtacaaagccttacctttggagtagagggaaggagagtcttaagtaaagctcacaaaaacccctaactcatagatgcagtctaaggaattcattccggaagcgacattagcatctcattagtgccattatgtgacttacttggcctctgagctagtctattttcttccctagaccccaaaatctcagtaatcctttggatccaagttatctccaagtgaaaataataggtttaggaaagcgtttttcttcaacttctatggagcacttgcttgctttgtcctatttgcatgtccgacggacggttctccagcaccactgctagtcgtcctccgcctgcctgggtacttgatcacaggatgcctctgacttctcctgcctttacccaagcaaaggattttccttgtcttcccacccaagagtgacggggctgacatgtgcccttgcctctaaatgatgaagctgaacctttgtctgggcaacttaacttaagaataagggagtcccaggcatgctctcccatcaataacaaattcagtgacatcagtttatgaatatatgaaatttgccaaagctctgtttagaccactgagtaactcacagctaggtttcaacttttcctttctaggttgtcttgggtttattgtaagagagcattatgaagaaaaaaatagatcataaagcttcttcaggaagctggtttcatatggtggtttagatttaaatagtgattgtctagcaccatttgaaatcagtgttcttgggggagaccagctgcgctgcactaccaacagcaaaagaagtgaatgggacagctctgaagtatttgaaagcaacagcaggatggctgtgagaacctgcctcacatgtagctgaccccttcctcacccctgccaacagtggtggcatatatcacaaatggcagtcaggtctctgcactggcggatccaactgtgatcgaaagttttccaaaaataagttgtgtctgtattgaacatgaacagactttcttcttgtcattattctctaacaatactgcataacaattatttgcatacatttgcattgcattaagtattctaagtaatctagagacgatttaaagtatacgggaggatgtgtgtaggttgtatgcaaatactacaccattttctatcagagacttgagcatctgtggattttggtatccaaggggctttctggaaccaatccctcaaggataccaagggatgaatgtaattgtacaggatatcgcattgttggaattttatacttctttgtggaataaacctatagcacttaatagatagtacagactcattccattgtgcctgggttaaagagcccaatgtatgctggatttagtaagatttgggccctcccaaccctcacgaccttctgtgaccccttagaggatgactgatttcttttggtgttcagagtcaatataattttctagcaccatctgaaatcggttataatgattggggaagagcaccatgatgctgactgctgagaggaaatgtattggtgaccgttggggccatggacaagaactaagaaaacaaatgcaaagcaataatgcaaaggtgatttttcttcttccagtttctaagttgaatttcactgacctgaattgcatgtggtataatactaacaaatggttcactattagcatatcatgaatggttatactttatagaaattccatagacttggtgggggttttgttttggtgacggatacctagaaacactcctggggaaaatcgatgactggcttagatgatgggaaaggagcagcgagggagtcaattctgttgttgatgagaagctgcaccagctatctctgaactctcctctcttagctggctgaggagttccctccatggttaaacaggtcattttcttacataaggaaaaatggtccagagaaactgggtttctatggctgagacagaactgtgctaatatgtgtcagttcagtttagaaggcaaagcaacaaaagcaggtgctacttgaactaggatcctagagtatacctttgatatggtttggatgtttgtcccctccaatctcatgttgaagtgtgatccccagtattgggggtggggcctggtgggggtggttgagtgatggggcggatcccttgtgaatggcttggtgatgtcctcacttaacgagttcctgctcagttagagggagccggttgtttaaaaaagctggcacctccttcctctctcttgctctttctttcttgctccctctctcgccatgtgacatgtctgctccctctccgccttccaccatgtataaaacatcctgaggcctcatcaggagccgagcaggtaccagtgccatgcttgtacggctgcaaaactgtaagccaaataaacctctttactatataaattacccagtctcgggtatgtctttatagcagcgcaaaacggactaacacaacctcactagaagtgaagtcattagaccaaaaacaatactgagtttttaggtccactaagaggcaaaaataaaaggctctttatagcagcatgatttataatcctttgggtataagcacatgtatgctagaacttaaagtataataataaaatataaaaataataaaataaaataaaaggctcaagaatgagccatctcggccgctcctcctccaggggcccctgtgtgcagctcccaacacaggtactgcacagaccggccacctgctgtccccatgtggcgtcctggagaactgcaggggcacctggtggaatttgggccttgtccttctgttttctttctggcctggaaaagccactggcctcacaatcaagagttaatattctatctaccctgcttaagctttcccgccctctcacactgcctccctccttttccctcctgcctgtcacttgtctggaaaaggccttttctcctcggggctaacacatgcaatttgtagccaaaacattctgtctaccacacaaatccaacgagaatcaggatcattccaccacatctttttctatcccatggttattattttcccaaaactcttgtcttttgctttcttacctattcgctgttttccctaagtaatcttatttactcccctggttccaaatacatccctatgtgttgatttccctcaaaccagaggtctggcctgggatattttttgagacatcttgctttttatatttttagtagctcctaagtataatagttcagcctgctcttctaaacttgtctcaatttaatccatttatgcttgaggttgcaattttttgaatttttgccatcagaccttggcaatgaccttgagcagtaggagacaactcccacatgcttagcattccaataatggaacactaggcatataccaaagccatccacattgccttctccccaaagccagctctccctgctctgccccatggtgaaagctgcgaacaccagaggtctgtgacctcttccctctcccttacccacatccatactcagcacaacttgttgctgttctctctcaattctctctttaggatccatccctttctctgctcttcctagacagctttctgggcccacccctcgccattagtctagctttgttatagccaccagttaatctttgtaaaatacatacgtgagctgggcacggtggctggtgcctgtaatcccagctactcaggaggctggggcaggaggttgcttgagcccaagaggttgaggctgcaatgagccatgattgcatcactgcactccagcctggacgacagaccaagatcccatctctttaaaaaataaaaataaaaattgtttaaccacatgaccttgctccttgtccctcacttttagctgaactcttataagtgtgtcaaccaggtaccagacccttcatgaccccatgcctacctgtgtcaccatcttctggctgtgcttcctggggcctgtgttctgtgctcacccagctgcttgctgctgagaacactcaggtatcttggcatttcttagcctgtgtctgtgctatttcctctgcccacatctcccctctcctcctcctcgtcagctagggacctccagcccctagggactgtaagctagggacctccaacctgcagcccctggctcaggtgtaaacttgtctaggaagccttcctctgccttctgcatcaaggcttagatccctgggcaaaggctttcttctctgtaccccttcagagcctgatggcacttcgcatgttatattctcatgattgattacctactaggcttctctgcaaggctggaaaatccttgctcctaggagggacggtagacaagaacaagacatactgtgtacactccattcttacattccacgtgagctatcctttgtgtaggtgtctgtggcaaatcttccttctaggctgcagcctctacttttcatagtttctctccattttcatctgccatgtgcatgaggaataaaaactacttccaatgatgaccaaaataagatggaagaaaataaccctgcttcaaagaacatttctgagaccataaatatgattttgatgtatcttccattatggattacccatgtggttttttttttccttctaaatgcataaacaggaattgtttaataggctagagagccatgaaaagcaaaataattaaggattgttgtcctcatttcatagaagagcaaacaaagacagaaaaacagatgtgccttaaccaaaaagggcagtaaatttatgcaaagcagactgaacccccattaaaagtaatcatttacagcgaccaagggccaagcacgtgatcaccaccctcctctcccaatagctctgaagggtgggtagtatttccccagcttatgcacgaggaaacagatgcacaggaaagtcaagtaacttgcctgcaatgccacggctagtcaacatgagagtttgaattacaaatcaggtggcggtggcggtggtgatgatgatgataatagcagcttatcatcatcattgagctcttattttgtgttagatacttggtaagtattttgcatgaattagctcatttagtctgactttcaaaacagccctttgaggtcagtgttattatcatcattcccattttgccatgaagaaacaaaagaattaaataatccaagatcgtctaactgacatttccaagcaggagcacaaacccagatctctgtgattccagagcctgtgctcttaaatcagcactgctcatttcagtgtgggagatggcgtaaatgggtgtgttcacttgctgggcagcctcatctgtggtccccttgatccccacctactggcattcatgccctgtgctattccctcccctggagtgtggactggacctagtaatttgcttctaatggatggaatatgtcaaaagtgatagatgtcacttctgagactaggttacaaatagactgtgattctgttgtcttcttcctcaggcttgcaccctcacgacatgctctctctctctccctctcctcttttctctcttctctcccttctctcctctctcttatctctctcttctctctctcgtacacatactctggaaagccagctaccatactgggaactgcccaatggagaggcccacattccacaaggaactgatgtcttcagccaatacctcgccaggacctgaggcctgctgagagtcacgagtggccttcgtgggtcctctctcaacgagcttgcaggtgaccactgcgccagccaaaaccttgatggcagcctatgagtgagtgaccctgagccagaagacccagttaagccatgcccaggatcctgacctgcagaaactatgacataataaatttattgttttatgccact

SEQ ID NO: 231 - DNA, 2062 bp, Homo sapiens
agctttctaaaatctctttaggggtgtgcgtaggctcctgtgtctatgcctgcttttgactgcccagttgaagcctcttcctatgccttttaaaatttcacgcactataaggaggaagagctcagggctcccaaaactttttatttagagggaagaatgctagggagatgggtatgcagagggttgaccaaattggaagaaaatatttattctgtagtttggtgttggaaaagggaattttccaatcagccacacctcagtgttgcggcaaaataattcttggctcccctggaaacgcatgggcaaggtagggcagagctgctgctgctgatactgccaccaccctgggcttcctgctgactctgggctactccctggggacaacagatttgcattgacgtccggggctgtccagaggccctcaagagccagttgtgagctgagcccagtatgggaaagatctaccttctggaagctactactacgtggtgcttggaaagaggactcaggagagtgcagcttgctctgtgagtgggtgacaacctcttggcgactcaggctcagctgaggatggtgccagtgtgccggagacagccgtcatactgccggatagagtggctcacttgcatgtatttggaacaaaaaaaggagatgcctggcagccccgctctctgcagtgctgttgagccaccaatttttgtggttttgtgaccacaagtgctgactgatgcgacatgaccccagtcttgtcagtgaatcatcaccaggctgcttactggaaactggatgcagcaaggaaataggatttaaccgctctctgcctcccaggagccctgaaatcagcattcccagaggaaagaagatggccatctgggcttggcttccggctccccccatctggctggaacacacatcagtcacccctgtgtaacctcctctgtgcctttcccatggagcactgtgtcatatcacaagtagaactacaagaagatatttctcctcagggcagaggctgggtcttccgattgaatctcccttctttcttcattgagatcctcttcttctggaagctggtttcacatggtggcttagatttttccatctttgtatctagcaccatttgaaatcagtgttttaggagtaagaattgcagcacagccaagggtggactgcagaggaactgctgctcatggaactggctcctctcctcttgccacttgagtctgttcgagaagtccagggaagaacttgaagagcaaaatacactcttgagtttgttgggttttgggagaggtgacagtagagaagggggttgtgtttaaaataaacacagtggcttgagcaggggcagaggttgtgatgctatttctgttgactcctagcagccatcaccagcatgaatgtgttcgtagggcctttgagtgtggcgattgtcatattctgttggataacaatgtattgggtgtcgattgtcatggggcaggggagagggcagtacacctggaggaccattttgtccacatcgacaccatcagtctgctcttagaggatgccctggagtattcggcgttgattgcggggcacccgaaatcagacttgccacctggactgtcgaggtgcagaccctgggagcaccactggcccatctcttacacaggctgaccgatttctcctggtgttcagagtctgtttttgtctagcaccatttgaaatcggttatgatgtagggggaaaagcagcagcctcgaagcctcatgccaactctgggcagcagcagcctgtggtttcctggaagatggatgggcagagaatagggaaggaagatcatgcttttccctactaacttctgtaactgcatgtatgatacattattgcagaggtaagagatagtttaatggatttttaaaaacaaattactataatttatctgatgttctctagttgcattttgctgaaatgtagtgctgttctaaattctgtaaattgattgctgttgaattatctttctgttgagaagagtctattcatgcatcctgaccttaataaatactatgttcagttt

SEQ ID NO: 232 - DNA, 2247 bp, Homo sapiens
agctttctaaaatctctttaggggtgtgcgtaggctcctgtgtctatgcctgcttttgactgcccagttgaagcctcttcctatgccttttaaaatttcacgcactataaggaggaagagctcagggctcccaaaactttttatttagagggaagaatgctagggagatgggtatgcagagggttgaccaaattggaagaaaatatttattctgtagtttggtgttggaaaagggaattttccaatcagccacacctcagtgttgcggcaaaataattcttggctcccctggaaacgcatgggcaaggtagggcagagctgctgctgctgatactgccaccaccctgggcttcctgctgactctgggctactccctggggacaacagatttgcattgacgtccggggctgtccagaggccctcaagagccagttgtgagctgagcccagtatgggaaagatctaccttctggaagctactactacgtggtgcttggaaagatgactcagtgtatcgggagccgggagatggagctctcagcagagtggccaggatgttctgtcaacatgaaagctacaaaaacagctcggggctctgagctgatccttagcctggggtcattttcagggagggctggacagagaaggcggccaccgcccgggcatcaggatgggagcgacaccagaggactcaggagagtgcagcttgctctgtgagtgggtgacaacctcttggcgactcaggctcagctgaggatggtgccagtgtgccggagacagccgtcatactgccggatagagtggctcacttgcatgtatttggaacaaaaaaaggagatgcctggcagccccgctctctgcagtgctgttgagccaccaatttttgtggttttgtgaccacaagtgctgactgatgcgacatgaccccagtcttgtcagtgaatcatcaccaggctgcttactggaaactggatgcagcaaggaaataggatttaaccgctctctgcctcccaggagccctgaaatcagcattcccagaggaaagaagatggccatctgggcttggcttccggctccccccatctggctggaacacacatcagtcacccctgtgtaacctcctctgtgcctttcccatggagcactgtgtcatatcacaagtagaactacaagaagatatttctcctcagggcagaggctgggtcttccgattgaatctcccttctttcttcattgagatcctcttcttctggaagctggtttcacatggtggcttagatttttccatctttgtatctagcaccatttgaaatcagtgttttaggagtaagaattgcagcacagccaagggtggactgcagaggaactgctgctcatggaactggctcctctcctcttgccacttgagtctgttcgagaagtccagggaagaacttgaagagcaaaatacactcttgagtttgttgggttttgggagaggtgacagtagagaagggggttgtgtttaaaataaacacagtggcttgagcaggggcagaggttgtgatgctatttctgttgactcctagcagccatcaccagcatgaatgtgttcgtagggcctttgagtgtggcgattgtcatattctgttggataacaatgtattgggtgtcgattgtcatggggcaggggagagggcagtacacctggaggaccattttgtccacatcgacaccatcagtctgctcttagaggatgccctggagtattcggcgttgattgcggggcacccgaaatcagacttgccacctggactgtcgaggtgcagaccctgggagcaccactggcccatctcttacacaggctgaccgatttctcctggtgttcagagtctgtttttgtctagcaccatttgaaatcggttatgatgtagggggaaaagcagcagcctcgaagcctcatgccaactctgggcagcagcagcctgtggtttcctggaagatggatgggcagagaatagggaaggaagatcatgcttttccctactaacttctgtaactgcatgtatgatacattattgcagaggtaagagatagtttaatggatttttaaaaacaaattactataatttatctgatgttctctagttgcattttgctgaaatgtagtgctgttctaaattctgtaaattgattgctgttgaattatctttctgttgagaagagtctattcatgcatcctgaccttaataaatactatgttcagttt

SEQ ID NO: 233 - DNA, 2329 bp, Homo sapiens
gaaagatgctctttctccaagacgcttgaccgctcttcctttcctggatggcaccagcagggccgattggagtggtaaaccctgggccggaaggcatgccaaagggtggacaggatggacaggagacagtagcacaacgaggagggggagaacagcggctgaattggaaatgataaaataaaatgaaattttaggagctcgctggctgggacaggcctggactgcaaggaggggtctttgcaccatctctgaaaagccgatgtgtatcctcagctttgagaactgaattccatgggttgtgtcagtgtcagacctctgaaattcagttcttcagctgggatatctctgtcatcgtgggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctactcgtttacatttattgattacttacgatgattcaggtactgttgtaagtgctttacatgctgttatacgagactcttgggagaaatcactttaatgaagcttgagacacatggcattgccatgcaatgatttttcccccctcttcacgggatcagagggaactaatagaatgtgacaatgattctttagcagggactgctgaggcttctggttcctttttaagatctgcagtgaaagaagatgagaaacatggatatgcccttcttttggtccccctcttcctttatttgatctctacttccttctataaatatattagggctacattgtccctttgtatttcaaacaaggcaaaaagaggttgtaattacactttactgcaatcctcagtttctccagggaacaggaatgcaaaggctttgaaggcctctctatttgctgacatggtcagctgggtgccatgggccaagtccttctgttgccctcctctgtcaccaagtaagctaggtcctttctgaggctcaggtttgctgtgatgatgatcacttttaggcagaaggttagaggcctcatgagtgctatatggactttattaggctttagatttgatggggaataagggatgtgatttgtcttttgggaactcatctttgattcatcattgtctcttggtatcttggaatttccatgtcattacagtctacagaatgaaagagtaacctgtcccagaggagaggcaggtgaaagactccacagcatgctcattctcattctgtcttctcagtgacaccgaggtttactgagtgcccactatgtgccaagcactgtgctcagggctttctttgtatgcatgatctcagtgaatctcaccaagcctcatctggaaaacggggacaaattaacaacaggatggcaaattgaaaaacacgtaaccatgttctacagatggaaaggggtgcttggttattatgaaggccccctcgcaagcgtgtgggacatgggtgtgttctctgggttgtactgatcagatcaaggacctcccccacccttctcacactctgcccacttccgccctttgcttatcagacccttagccagtgactcattccagaaccagaaccttggtgaaatctcaaccgacaccagagatcggtgtcttcagtcctagactgatggagaaaatccagaatatatactagaagctccaaatgctctgggtttcagctcctctgtgctgtggacactgactttggctcagaactccgatttagtacaaaaggctcatttttatttcaggggcactcttcctaaagcaaacctaataaatgaaatatggaattcacagatacacacacacattaaaaaattaacctagtgtatctgtgaggagtaggcagaaattcactgtataaaagaatgcttcatttcatagagaatttgtgttaagattccattagatagtacatttctcaaagatttttgaggttgtatttgctttaccaaaacttggtttatgtaagtggaaaaagcatgttgcaaaataacttggtgtctatgattcagtttatgtaaaataataaatgtatgtaggaatacgtgtgttgaaagatgtacatcaatttgctaacaatggttatctctgacgtggtgggatttgagatgtgtttttctttttggttgtatttttctctattgtttgacttaacacagaacatgtttggttacaacaataaagttattgaagac

(19) United States
(12) Patent Application Publication
(10) Pub. No.: US 2010/0298407 A1
(43) Pub. Date: Nov. 25, 2010

(54) COMPOSITIONS AND METHODS FEATURING MICRONAS FOR TREATING NEOPLASIA

(75) Inventors: Joshua T. Mendell, Baltimore, MD (US); Andrei Thomas-Tikhonenko, Philadelphia, PA (US); Tsung-Cheng Chang, Baltimore, MD (US); Duonan Yu, Philadelphia, PA (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, Baltimore, MD (US)

(21) Appl. No.: 12/523,431

(22) PCT Filed: Jan. 17, 2008
(86) PCT No.: PCT/US08/00656

§ 371 Date: Aug. 11, 2010

Related U.S. Application Data
(60) Provisional application No. 60/880,919, filed on Jan. 17, 2007.

(51) Int. Cl. C07H 21/02; C12N 15/63; A61K 31/7105; C12N 5/00; C12N 5/09; C12Q 1/68; C40B 40/06

(52) U.S. Cl. 514/44 A; 536/23.1; 435/320.1; 435/375; 435/366; 435/6; 506/16

(57) ABSTRACT

The invention provides compositions and methods for the treatment of a neoplasia. The methods of the invention involve expressing a microRNA usually repressed by Myc in a cell of a subject diagnosed as having a neoplasia.

### CROSS-REFERENCE TO RELATED APPLICATION

This application claims the benefit of the following U.S. Provisional Application No. 60/880,919, filed on Jan. 17, 2007, the entire contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: R01CA120185, R01CA122334, and R01CA102709. The government has certain rights in the invention.

### BACKGROUND OF THE INVENTION

Dysregulated expression or function of the Myc oncogenic transcription factor occurs frequently in human malignancies. Through the positive and negative regulation of an expansive network of target genes, Myc globally reprograms cells to drive proliferation and in some settings induce cell death. Myc utilizes distinct mechanisms for activating and repressing gene expression. When inducing transcription, Myc dimerizes with its binding partner Max and binds to genomic DNA directly upstream or within the first intron of target genes. When repressing transcription, Myc does not appear to contact DNA directly. Rather, Myc is recruited to core promoters via protein-protein interactions where it antagonizes the activity of positive regulators of transcription. For example, Myc can bind to and inhibit the activity of the transcription factor Myc-interacting zinc finger protein 1 (Miz1), thus preventing Miz1 from activating transcription of the CDKN1A (p21 WAF1/CIP1) and CDKN2B (p15INK4b) cell-cycle-inhibitory genes. Repression of other Myc targets is likely mediated through the ability of Myc to interact with and antagonize the activity of additional proteins including Sp1, Smad2, and NF—Y.

MicroRNAs (miRNAs) are a diverse family of ˜18-24 nucleotide RNA molecules that have recently emerged as a novel class of Myc-regulated transcripts. miRNAs regulate the stability and translational efficiency of partially-complementary target messenger RNAs (mRNAs). miRNAs are initially transcribed by RNA polymerase II (pol II) as long primary transcripts (pri-microRNAs) that are capped, polyadenylated, and frequently spliced. The mature microRNA sequences are located in introns or exons of pri-microRNAs, within regions that fold into ˜60-80 nucleotide hairpin structures. While the majority of pri-microRNAs are noncoding transcripts, a subset of microRNAs are located within introns of protein-coding genes. microRNA maturation requires a series of endonuclease reactions in which microRNA hairpins are excised from pri-miRNAs, the terminal loop of the hairpin is removed, and one strand of the resulting duplex is selectively loaded into the RNA-induced silencing complex (RISC). This microRNA-programmed RISC is the effector complex which carries out target mRNA regulation.

A large body of evidence has documented nearly ubiquitous dysregulation of miRNA expression in cancer cells. These miRNA expression changes are highly informative for cancer classification and prognosis. Moreover, altered expression of specific miRNAs has been demonstrated to promote tumorigenesis. For example, a group of six co-transcribed miRNAs known as the mir-17 cluster is amplified in lymphoma and solid tumors. These miRNAs are frequently overexpressed in tumors, promote proliferation in cell lines, and accelerate angiogenesis and tumorigenesis in mouse models of Myc-induced colon cancer and lymphoma. Although select miRNAs are upregulated in cancer cells, global miRNA abundance appears to be generally reduced in tumors. miRNA downregulation likely contributes to neoplastic transformation by allowing the increased expression of proteins with oncogenic potential. Recent evidence suggests that a block in the first step of miRNA processing may contribute to the reduced abundance of select miRNAs in cancer cells. Cancer causes one in every four US deaths and is the second leading cause of death among Americans. Additional mechanisms of miRNA downregulation, including direct transcriptional repression, have not yet been investigated. Improved compositions and methods for the treatment or prevention of neoplasia are required.

### SUMMARY OF THE INVENTION

As described below, the present invention provides compositions featuring microRNAs and methods of using them for the treatment of neoplasia.

In one aspect, the invention generally provides an isolated oligonucleotide containing a nucleobase sequence having at least 85%, 90%, 95%, 97%, 99% or 100% identity to the sequence of a microRNA that is any one or more of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a116-1, or any other nucleic acid molecule delineated herein, or a fragment thereof, where expression of the microRNA in a neoplastic cell reduces the survival of the cell or reduces cell division.

In another aspect, the invention provides an isolated nucleic acid molecule encoding an oligonucleotide delineated herein, where expression of the oligonucleotide in a neoplastic cell reduces the survival of the cell or reduces cell division.

In another aspect, the invention features an expression vector encoding a nucleic acid molecule delineated herein, where the nucleic acid molecule is positioned for expression in a mammalian cell (e.g., a human cell, such as a neoplastic cell). In one embodiment, the vector is a viral vector selected from the group consisting of a retroviral, adenoviral, lentiviral and adeno-associated viral vector.

In a related aspect, the invention features a host cell (e.g., a human cell, such as a neoplastic cell) containing the expression vector of a previous aspect or a nucleic acid molecule delineated herein.

In another aspect, the invention features a pharmaceutical composition for the treatment of a neoplasia (e.g., lymphoma), the composition containing an effective amount of an oligonucleotide having at least 85%, 90%, 95%, 97%, 99% or 100% identity to the sequence of a microRNA that is any one or more of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a116-1 and a pharmaceutically acceptable excipient, where expression of the microRNA in a neoplastic cell reduces the survival of the cell or reduces cell division. In one embodiment, the amount of microRNA is sufficient to reduce the survival or proliferation of a neoplastic cell by at least about 5%, 10%, 25%, 50%, 75%, or 100% relative to an untreated control cell. In one embodiment, the composition contains at least one of miR-22, miR-26a, miR-34a, miR-150, miR-195/497, or miR-15a/16-1.

In another aspect, the invention features a pharmaceutical composition for the treatment of a neoplasia, the composition containing an effective amount of an expression vector encoding a microRNA that is any one or more of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1 and a pharmaceutically acceptable excipient, where expression of the microRNA in a neoplastic cell reduces the survival of the cell or reduces cell division. In one embodiment, the amount of microRNA is sufficient to reduce expression of Myc in a neoplastic cell by at least about 5%, 10%, 25%, 50%, 75%, or 100% relative to an untreated control cell.

In another aspect, the invention provides a method of reducing the growth, survival or proliferation of a neoplastic cell, the method involving contacting the cell (e.g., human cell, such as a neoplastic cell) with an oligonucleotide containing a nucleobase sequence having at least 85%, 90%, 95%, 97%, 99% or 100% identity to a microRNA that is any one or more of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1, thereby reducing the growth, survival or proliferation of a neoplastic cell relative to an untreated control cell.

In another aspect, the invention features a method of reducing the growth, survival or proliferation of a neoplastic cell, the method involving contacting the cell with an expression vector encoding a microRNA that is any one or more of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1, thereby reducing the growth, survival or proliferation of a neoplastic cell relative to an untreated control cell.

In another aspect, the invention features a method of treating neoplasia (e.g., lymphoma) in a subject (e.g., a human or veterinary patient), the method involving administering to the subject an effective amount of an oligonucleotide containing a nucleobase sequence having at least 85%, 90%, 95%, 97%, 99% or 100% identity to a microRNA that is any one or more of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1, thereby treating a neoplasia in the subject.

In another aspect, the invention features a method of treating neoplasia in a subject (e.g., a human or veterinary patient), the method involving administering to the subject an effective amount of an expression vector encoding a microRNA that is any one or more of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3 7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1, thereby treating the neoplasia in the subject.

In another aspect, the invention features a method of characterizing a neoplasia, the method involving assaying the expression of a microRNA that is any one or more of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1. In one embodiment, the method involves assaying the expression of a combination of microRNAs, e.g., two, three, four, five, or more of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1. In one embodiment, the neoplasia is characterized as having Myc disregulation (e.g., having an increase in the expression of a microRNA that is repressed by Myc in a control cell).

In yet another aspect, the invention features method of identifying an agent for the treatment of a neoplasia, the method involving contacting a neoplastic cell with a candidate agent; and assaying the expression of a microRNA that is any one or more of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1, where an increase in the microRNA expression identifies the agent as useful for the treatment of a neoplasia. In one embodiment, the method further involves testing the agent in a functional assay (e.g., an assay that determines cell growth, proliferation, or survival relative to an untreated control cell).

In another aspect, the invention features a primer set containing at least two pairs of oligonucleotides, each of which pair binds to a microRNA that is any one or more of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1 or a fragment thereof.

In another aspect, the invention features a probe set containing at least two oligonucleotides that binds to at least two microRNAs that are any of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a116-1 or a fragment thereof.

In another aspect, the invention features a microarray containing a microRNA or nucleic acid molecule encoding a microRNA that is miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1 or a fragment thereof.

In various embodiments of any of the above aspects, the oligonucleotide contains the nucleobase sequence of the microRNA. In another embodiment, the oligonucleotide consists essentially of the nucleobase sequence of the microRNA. In various embodiments of any of the above aspects, the microRNA sequence is a pri-microRNA, mature or hairpin form. In other embodiments, the oligonucleotide contains at least one modified linkage (e.g., phosphorothioate, methylphosphonate, phosphotriester, phosphorodithioate, and phosphoselenate linkages), contains at least one modified sugar moiety or one modified nucleobase. In various embodiments of any method or composition described herein, the nucleic acid molecule consists essentially of the nucleotide sequence encoding a mature or hairpin form of a microRNA (e.g., miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a116-1) or a fragment or analog thereof. In other embodiments, the microRNA is any one or more of miR-22, miR-26a, miR-34a, miR-150, miR-195/497, and miR-15a/16-1. In still other embodiments of any of the above aspects, the composition contains two, three, four, five, or six microRNAs (e.g., miR-22, miR-26a, miR-34a, miR-150, miR-195/497, and miR-15a/16-1). In still other embodiments, the oligonucleotide contains a modification (e.g., a modification described herein, such as a modification that enhances nuclease resistance). In various embodiments of the invention, the cell is a mammalian cell (e.g., a human cell, a neoplastic cell, or a lymphoma cell). In various embodiments of the above aspects, the composition or method disrupts the cell cycle or induces apoptosis in a neoplastic cell. In various embodiments of the above aspects, the method reduces cell division, cell survival or increases expression of Myc in a neoplastic cell by at least about 5%, 10%, 25%, 50%, 75%, or 100% relative to an untreated control cell. In various embodiments, the subject is contacted with two, three, four, five, or six microRNAs (e.g., miR-22, miR-26a, miR-34a, miR-150, miR-195/497, and miR-15a/16-1).

The invention provides for the treatment of neoplasia by expressing microRNAs usually repressed by Myc. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The sequence of microRNAs referred to herein is known in the art. In particular, the sequence of microRNAs is publically available via miRBase (http://microrna.sanger.ac.uk/), which provides microRNA data. Each entry in the miRBase Sequence database represents a predicted hairpin portion of a miRNA transcript, with information on the location and sequence of the mature miRNA sequence. Both hairpin and mature sequences are available for searching using BLAST and SSEARCH, and entries can also be retrieved by name, keyword, references and annotation.

By “miR-15a microRNA” is meant a nucleic acid molecule comprising a nucleobase sequence that is substantially identical to the sequence of hsa-mir-15a, MirBase Reference No. MI0000069, MIMAT0000068, or a fragment thereof whose expression reduces the growth of a neoplasia. Exemplary miR-15a microRNA sequences follow:

CCUUGGAGUAAAGUAGCAGCACAUAAUGGUUUGUGGAUUUUGAAAAGGUG
CAGGCCAUAUUGUGCUGCCUCAAAAAUACAAGG (hairpin) and
14-uagcagcacauaaugguuugug-35 (mature).

By “miR-15a gene” is meant a polynucleotide that encodes a miR-15a microRNA or analog thereof.

By “mir16-1 microRNA” is meant a nucleic acid molecule comprising a nucleobase sequence that is substantially identical to the sequence of hsa-mir-16-1, MirBase Reference No. MI0000070, MIMAT0000069, or a fragment thereof whose expression reduces the growth of a neoplasia. Exemplary mir16-1 microRNA sequences follow:

GUCAGCAGUGCCUUAGCAGCACGUAAAUAUUGGCGUUAAGAUUCUAAAAU
UAUCUCCAGUAUUAACUGUGCUGCUGAAGUAAGGUUGAC (hairpin)
or 14-uagcagcacguaaauauuggcg-35 (mature).

Human miR-16 and miR-15a are clustered within 0.5 kb at 13q14. This region has been shown to be deleted in many B cell chronic lymphocytic leukemias (CLL). A second putative mir-16 hairpin precursor is located on chromosome 3 (MI0000738).

By “mir16-1 gene” is meant a polynucleotide that encodes a mir16-1 microRNA or fragment thereof.

By “mir-22 microRNA” is meant a nucleic acid molecule comprising a nucleobase sequence that is substantially identical to the sequence of NCBI Reference No. AJ421742, MirBase Reference No. MI0000078 or MIMAT0000077, or a fragment thereof whose expression reduces the growth of a neoplasia. The sequence of exemplary mir-22 microRNAs follows:

53-Aagcugccaguugaagaacugu-74 (mature)
GGCUGAGCCGCAGUAGUUCUUCAGUGGCAAGCUUUAUGUCCUGACCCAGC
UAAAGCUGCCAGUUGAAGAACUGUUGCCCUCUGCC (hairpin).

By “mir-22 gene” is meant a polynucleotide encoding a mir-22 microRNA. The sequence of an exemplary mir-22 gene is provided at NCBI Reference No. AF480525.

By “miR-26a-1 microRNA” is meant a nucleic acid molecule comprising a nucleobase sequence that is substantially identical to the sequence of hsa-mir-26a-1, MirBase Accession No. MI0000083, MIMAT0000082, or a fragment thereof whose expression reduces the growth of a neoplasia. The sequence of two exemplary mir-26a-1 microRNAs follow:

10-uucaaguaauccaggauaggcu-31 (mature); and
GUGGCCUCGUUCAAGUAAUCCAGGAUAGGCUGUGCAGGUCCCAAUGGGCC
UAUUCUUGGUUACUUGCACGGGGACGC (hairpin).

By “miR-26a-1 gene” is meant a polynucleotide encoding a mir-26a-1 microRNA or an analog thereof.

By “miR-26a-2 microRNA” is meant a nucleic acid molecule comprising a nucleobase sequence that is substantially identical to the sequence of hsa-mir-26a-2, MirBase Accession No. MI0000750, MIMAT0000082, or a fragment thereof whose expression reduces the growth of a neoplasia. The sequence of two exemplary miR-26a-2 microRNA follows:

14-uucaaguaauccaggauaggcu-35 (mature) or
GGCUGUGGCUGGAUUCAAGUAAUCCAGGAUAGGCUGUUUCCAUCUGUGAG
GCCUAUUCUUGAUUACUUGUUUCUGGAGGCAGCU (hairpin).

By “miR-26a-2 gene” is meant a polynucleotide encoding a miR-26a-2 microRNA or an analog thereof.

By “mir-29a microRNA” is meant a nucleic acid molecule comprising a nucleobase sequence that is substantially identical to the sequence of hsa-mir-29a. Exemplary mir-29a sequences are provided at Mirbase Accession No. MI0000087 and MIMAT0000086. The sequence of two exemplary mir-29a microRNAs follows:

AUGACUGAUUUCUUUUGGUGUUCAGAGUCAAUAUAAUUUUCUAGCACCAU
CUGAAAUCGGUUAU (hairpin) and UAGCACCAUCUGAAAUCGGU
UA (mature).

By “mir-29a gene” is meant a polynucleotide encoding a mir-29a microRNA.

By “miR-29b-1 microRNA” is meant a nucleic acid molecule comprising a nucleobase sequence that is substantially identical to the sequence of hsa-mir-29b-1. Exemplary mir-29b-1 sequences are provided at Mirbase Accession No. MI0000105, hsa-miR-29b MIMAT0000100, or a fragment thereof. The sequence of two exemplary miR-29b-1 microRNAs follows:

UAGCACCAUUUGAAAUCAGUGUU (mature), and
CUUCAGGAAGCUGGUUUCAUAUGGUGGUUUAGAUUUAAAUAGUGAUUGUC
UAGCACCAUUUGAAAUCAGUGUUCUUGGGGG hairpin.

By “miR-29b-2 microRNA” is meant a nucleic acid molecule comprising a nucleobase sequence that is substantially identical to the sequence of hsa-mir-29b-2, MirBase Accession No. MI0000107, MIMAT0000100, or a fragment thereof whose expression reduces the growth of a neoplasia. The sequence of two exemplary miR-29b-2 microRNAs follows:

52-uagcaccauuugaaaucaguguu-74 (mature) or
CUUCUGGAAGCUGGUUUCACAUGGUGGCUUAGAUUUUUCCAUCUUUGUAU
CUAGCACCAUUUGAAAUCAGUGUUUUAGGAG (hairpin).

By “miR-29b-2 gene” is meant a polynucleotide encoding a miR-29b-2 microRNA or an analog thereof.

By “miR-29c microRNA” is meant a nucleic acid molecule comprising a nucleobase sequence that is substantially identical to the sequence of hsa-miR-29c, MirBase Accession No. MI0000735, MIMAT0000681, or a fragment thereof whose expression reduces the growth of a neoplasia. The sequence of two exemplary miR-29c microRNAs follows:

54-uagcaccauuugaaaucgguua-75 (mature) or
AUCUCUUACACAGGCUGACCGAUUUCUCCUGGUGUUCAGAGUCUGUUUUU
GUCUAGCACCAUUUGAAAUCGGUUAUGAUGUAGGGGGA (hairpin).

By “miR-29c gene” is meant a polynucleotide encoding a mir-29c microRNA or analog thereof.

By “miR-30e microRNA” is meant a nucleic acid molecule comprising a nucleobase sequence that is substantially identical to the sequence of hsa-mir-30e, MirBase Accession No. MI0000749, MIMAT0000692, or a fragment thereof whose expression reduces the growth of a neoplasia. The sequence of two exemplary miR-30e microRNA follows:

17-uguaaacauccuugacuggaag-38 (mature)
or
GGGCAGUCUUUGCUACUGUAAACAUCCUUGACUGGAAGCUGUAAGGUG
UUCAGAGGAGCUUUCAGUCGGAUGUUUACAGCGGCAGGCUGCCA
(hairpin).

By “miR-30e gene” is meant a polynucleotide that encodes a miR-30e microRNA.

By “miR-30c-1 microRNA” is meant a nucleic acid molecule comprising a nucleobase sequence that is substantially identical to the sequence of hsa-mir-30c-1 MirBase Accession No. MI0000736, MIMAT0000244, or a fragment thereof whose expression reduces the growth of a neoplasia. The sequence of two exemplary miR-30c-1 microRNAs follows:

17-uguaaacauccuacacucucagc-39 (mature)
or
ACCAUGCUGUAGUGUGUGUAAACAUCCUACACUCUCAGCUGUGAGCUC
AAGGUGGCUGGGAGAGGGUUGUUUACUCCUUCUGCCAUGGA
(hairpin).

By “miR-30c-1 gene” is meant a polynucleotide that encodes a miR-30c-1 microRNA or an analog thereof.

By “miR-26b microRNA” is meant a nucleic acid molecule comprising a nucleobase sequence that is substantially identical to the sequence of hsa-mir-26b, MirBase Accession No. MI0000084, MIMAT0000083, or a fragment thereof whose expression reduces the growth of a neoplasia. The sequence of exemplary hsa-mir-26b microRNAs follows:

CCGGGACCCAGUUCAAGUAAUUCAGGAUAGGUUGUGUGCUGUCCAG
CCUGUUCUCCAUUACUUGGCUCGGGGACCGG (hairpin)
or
12-uucaaguaauucaggauaggu-32 (mature).

By “miR-26b gene” is meant a polynucleotide encoding a miR-26b microRNA or analog thereof.

By “miR-30c-2 microRNA” is meant a nucleic acid molecule comprising a nucleobase sequence that is substantially identical to the sequence of hsa-mir-30c-2, MirBase Accession No. MI0000254, MIMAT0000244, or a fragment thereof whose expression reduces the growth of a neoplasia. The sequence of an exemplary miR-30c-2 microRNA follows:

AGAUACUGUAAACAUCCUACACUCUCAGCUGUGGAAAGUAAGAAAGCUG
GGAGAAGGCUGUUUACUCUUUCU (hairpin), 7-
uguaaacauccuacacucucagc-29 (mature),
or
47-cugggagaaggcuguuuacucu-68
(minor alternative processing).

By “miR-30c gene” is meant a polynucleotide that encodes a miR-30c microRNA or analog thereof.

By “miR-34a microRNA” is meant a nucleic acid molecule comprising a nucleobase sequence that is substantially identical to the sequence of hsa-mir-34a MirBase Accession No. MI0000268, MIMAT0000255, or a fragment thereof whose expression reduces the growth of a neoplasia. Exemplary miR-34a microRNA sequences follow:

GGCCAGCUGUGAGUGUUUCUUUGGCAGUGUCUUAGCUGGUUGUUGUGA
GCAAUAGUAAGGAAGCAAUCAGCAAGUAUACUGCCCUAGAAGUGCUGC
ACGUUGUGGGGCCC (hairpin)
or
22-uggcagugucuuagcugguugu-43 (mature).

By “miR-34a gene” is meant a polynucleotide that encodes a miR-34a microRNA or analog thereof.

By “miR-146a microRNA” is meant a nucleic acid molecule comprising a nucleobase sequence that is substantially identical to the sequence of hsa-mir-146a, MirBase Accession No. MI0000477, MIMAT0000449, or a fragment thereof whose expression reduces the growth of a neoplasia. The sequence of two exemplary miR-146a microRNA follows:

21-ugagaacugaauuccauggguu-42 (mature)
or
CCGAUGUGUAUCCUCAGCUUUGAGAACUGAAUUCCAUGGGUUGUGUCAG
UGUCAGACCUCUGAAAUUCAGUUCUUCAGCUGGGAUAU
CUCUGUCAUCGU (hairpin).

By “miR-146a gene” is meant a polynucleotide encoding a miR-146a microRNA or analog thereof.

By “miR-150 microRNA” is meant a nucleic acid molecule comprising a nucleobase sequence that is substantially identical to the sequence of hsa-mir-150 MirBase Accession No. MI0000479, MIMAT0000451, or a fragment thereof whose expression reduces the growth of a neoplasia. The sequence of two exemplary miR-150 microRNAs follows:

16-ucucccaacccuuguaccagug-37 (mature)
or
CUCCCCAUGGCCCUGUCUCCCAACCCUUGUACCAGUGCUGGGCUCAG
ACCCUGGUACAGGCCUGGGGGACAGGGACCUGGGGAC (hairpin).

By “miR-150 gene” is meant a polynucleotide encoding a miR-150 microRNA or analog thereof.

By “miR-195 microRNA” is meant a nucleic acid molecule comprising a nucleobase sequence that is substantially identical to the sequence of hsa-mir-195, MirBase Accession No. MI0000489, MIMAT0000461, or a fragment thereof whose expression reduces the growth of a neoplasia. Exemplary miR-195 microRNA sequences follow:

AGCUUCCCUGGCUCUAGCAGCACAGAAAUAUUGGCACAGGGAAGCGAGU
CUGCCAAUAUUGGCUGUGCUGCUCCAGGCAGGGUGGUG (hairpin)
and
15-uagcagcacagaaauauuggc-35 (mature).

By “miR-195 gene” is meant a polynucleotide encoding a miR-195 microRNA or analog thereof.

By “miR-497 microRNA” is meant a nucleic acid molecule comprising a nucleobase sequence that is substantially identical to the sequence of hsa-mir-497, MirBase Accession No. MI0003138, MIMAT0002820, or a fragment thereof whose expression reduces the growth of a neoplasia. Exemplary miR-497 microRNA sequences follow:

CCACCCCGGUCCUGCUCCCGCCCCAGCAGCACACUGUGGUUUGUAC
GGCACUGUGGCCACGUCCAAACCACACUGUGGUGUUAGAGCGAGGGU
GGGGGAGGCACCGCCGAGG (hairpin)
and
24-cagcagcacacugugguuugu-44 (mature).

By “miR-497 gene” is meant a polynucleotide encoding a miR-497 microRNA or analog thereof.

By “let-7a-1 microRNA” is meant a nucleic acid molecule comprising a nucleobase sequence that is substantially identical to the sequence of hsa-let-7a-1, MirBase Accession No. MI0000060, MIMAT0000062, or a fragment thereof whose expression reduces the growth of a neoplasia. The sequence of two exemplary let-7a-1 microRNAs follow:

6-ugagguaguagguuguauaguu-27 (mature)
or
UGGGAUGAGGUAGUAGGUUGUAUAGUUUUAGGGUCACACCCACCACUGG
GAGAUAACUAUACAAUCUACUGUCUUUCCUA (hairpin).

By “let-7a-1 gene” is meant a polynucleotide encoding a let-7a-1 microRNA or analog thereof.

By “let-7f-1 microRNA” is meant a nucleic acid molecule comprising a nucleobase sequence that is substantially identical to the sequence of hsa-let-7f-1 MirBase Accession No. MI0000067, MIMAT0000067, or a fragment thereof whose expression reduces the growth of a neoplasia. The sequence of two exemplary let-7f-1 microRNAs follows:

7-ugagguaguagauuguauaguu-28 (mature)
or
UCAGAGUGAGGUAGUAGAUUGUAUAGUUGUGGGGUAGUGAUUUUACCCUG
UUCAGGAGAUAACUAUACAAUCUAUUGCCUUCCCUGA (hairpin).

By “let-7f-1 gene” is meant a polynucleotide encoding a let-7f-1 microRNA or analog thereof.

By “let-7d microRNA” is meant a nucleic acid molecule comprising a nucleobase sequence that is substantially identical to the sequence of hsa-let-7d, MirBase Accession No. MI0000065, MIMAT0000065, or a fragment thereof whose expression reduces the growth of a neoplasia. The sequence of two exemplary let-7d microRNAs follows:

AGAGGUAGUAGGUUGCAUAGUU (mature)
or
CCUAGGAAGAGGUAGUAGGUUGCAUAGUUUUAGGGCAGGGAUUUUGCCCA
CAAGGAGGUAACUAUACGACCUGCUGCCUUUCUUAGG (hairpin).

By “let-7d gene” is meant a polynucleotide encoding a let-7d microRNA or analog thereof.

By “miR-100 microRNA” is meant a nucleic acid molecule comprising a nucleobase sequence that is substantially identical to the sequence of hsa-mir-100, MirBase Accession No. MI0000102, MIMAT0000098, or a fragment thereof whose expression reduces the growth of a neoplasia. The sequence of two exemplary miR-100 microRNAs follows:

13-aacccguagauccgaacuugug-34 (mature)
CCUGUUGCCACAAACCCGUAGAUCCGAACUUGUGGUAUUAGUCCGCACA
AGCUUGUAUCUAUAGGUAUGUGUCUGUUAGG (hairpin).

By “miR-100 gene” is meant a polynucleotide encoding a miR-100 microRNA or analog thereof.

By “let-7a-2 microRNA” is meant a nucleic acid molecule comprising a nucleobase sequence that is substantially identical to the sequence of MirBase Accession No MI0000061, MIMAT0000062, or a fragment thereof whose expression reduces the growth of a neoplasia. The exemplary sequences of let-7a-2 microRNAs follow:

AGGUUGAGGUAGUAGGUUGUAUAGUUUAGAAUUACAUCAAGGGAGAUA
ACUGUACAGCCUCCUAGCUUUCCU (hairpin)
and
5-ugagguaguagguuguauaguu-26 (mature).

By “let-7a-2 gene” is meant a polynucleotide encoding a let-7a-2 microRNA or analog thereof.

By “miR-125b-1 microRNA” is meant a nucleic acid molecule comprising a nucleobase sequence that is substantially identical to the sequence of hsa-mir-125b-1, MirBase Accession No. MI0000446, MIMAT0000423, or a fragment thereof whose expression reduces the growth of a neoplasia. The exemplary sequences of hsa-mir-125b-1 microRNAs follow:

15-ucccugagacccuaacuuguga-36 (mature)
or
UGCGCUCCUCUCAGUCCCUGAGACCCUAACUUGUGAUGUUUACCGUUUAA
AUCCACGGGUUAGGCUCUUGGGAGCUGCGAGUCGUGCU (hairpin).

By “let-7a-3 microRNA” is meant a nucleic acid molecule comprising a nucleobase sequence that is substantially identical to the sequence of hsa-let-7a-3, MirBase Accession No. MI0000062, MIMAT0000062, or a fragment thereof whose expression reduces the growth of a neoplasia. The sequence of two exemplary let-7a-3 microRNA follows:

GGGUGAGGUAGUAGGUUGUAUAGUUUGGGGCUCUGCCCUGC
UAUGGGAUAACUAUACAAUCUACUGUCUUUCCU (hairpin)
or
4-ugagguaguagguuguauaguu-25.

By “let-7b microRNA” is meant a nucleic acid molecule comprising a nucleobase sequence that is substantially identical to the sequence of hsa-let-7b MirBase Accession No. MI0000063, MIMAT0000063, or a fragment thereof whose expression reduces the growth of a neoplasia. The sequence of two exemplary let-7b microRNAs follows:

6-ugagguaguagguugugugguu-27 (mature)
or
CGGGGUGAGGUAGUAGGUUGUGUGGUUUCAGGGCAGUGAUGUUGCCCCUC
GGAAGAUAACUAUACAACCUACUGCCUUCCCUG (hairpin).

By “let-7b gene” is meant a polynucleotide encoding a let-7b microRNA or analog thereof.

By “miR-99a microRNA” is meant a nucleic acid molecule comprising a nucleobase sequence that is substantially identical to the sequence of hsa-mir-99a, MirBase Accession No. MI0000101, MIMAT0000097, or a fragment thereof whose expression reduces the growth of a neoplasia. The sequence of exemplary miR-99a microRNAs follows:

CCCAUUGGCAUAAACCCGUAGAUCCGAUCUUGUGGUGAAGUGGACCGCAC
AAGCUCGCUUCUAUGGGUCUGUGUCAGUGUG (hairpin)
or
13-aacccguagauccgaucuugug-
34 (mature).

By “miR-99a gene” is meant a polynucleotide encoding a miR-99a microRNA or analog thereof.

By “let-7c microRNA” is meant a nucleic acid molecule comprising a nucleobase sequence that is substantially identical to the sequence of hsa-let-7c MirBase Accession No. MI0000064, MIMAT0000064, or a fragment thereof whose expression reduces the growth of a neoplasia. The sequences of exemplary let-7c microRNAs follows:

GCAUCCGGGUUGAGGUAGUAGGUUGUAUGGUUUAGAGUUACACCCUGGGA
GUUAACUGUACAACCUUCUAGCUUUCCUUGGAGC (hairpin)
or
11-ugagguaguagguuguaugguu-32 (mature).

By “let-7c gene” is meant a polynucleotide that encodes a let-7c microRNA or an analog thereof.

By “miR-125b-2 microRNA” is meant a nucleic acid molecule comprising a nucleobase sequence that is substantially identical to the sequence of hsa-mir-125b-2, MirBase Accession No. MI0000470, MIMAT0000423, or a fragment thereof, whose expression reduces the growth of a neoplasia. The sequences of exemplary miR-125b-2 microRNAs follow:

ACCAGACUUUUCCUAGUCCCUGAGACCCUAACUUGUGAGGUAUUUUAGUA
ACAUCACAAGUCAGGCUCUUGGGACCUAGGCGGAGGGGA (hairpin)
or
17-ucccugagacccuaacuuguga-38 (mature).

By “miR-99b microRNA” is meant a nucleic acid molecule comprising a nucleobase sequence that is substantially identical to the sequence of hsa-mir-99b, MirBase Accession No. MI0000746, MIMAT0000689, or a fragment thereof, whose expression reduces the growth of a neoplasia. The sequence of an exemplary miR-99b microRNA follows:

GGCACCCACCCGUAGAACCGACCUUGCGGGGCCUUCGCCGCACACAAGCU
CGUGUCUGUGGGUCCGUGUC (hairpin)
or
7-cacccguagaaccgaccuugcg-28 (mature).

By “miR-99b gene” is meant a polynucleotide that encodes a miR-99b microRNA.

By “let-7e microRNA” is meant a nucleic acid molecule comprising a nucleobase sequence that is substantially identical to the sequence of hsa-let-7e MI0000066, MIMAT0000066, or a fragment thereof, whose expression reduces the growth of a neoplasia. The sequence of exemplary let-7e microRNAs follows:

CCCGGGCUGAGGUAGGAGGUUGUAUAGUUGAGGAGGACACCCAAGGAGAU
CACUAUACGGCCUCCUAGCUUUCCCCAGG (hairpin)
or
8-Ugagguaggagguuguauaguu-29 (mature).

By “let-7e gene” is meant a polynucleotide encoding a let-7e microRNA or analog thereof.

By “miR-125a microRNA” is meant a nucleic acid molecule comprising a nucleobase sequence that is substantially identical to the sequence of hsa-mir-125a, MirBase Accession No. MI0000469, MIMAT0000443, MIMAT0004602, or a fragment thereof, whose expression reduces the growth of a neoplasia. The sequence of exemplary miR-125a microRNAs follows:

UGCCAGUCUCUAGGUCCCUGAGACCCUUUAACCUGUGAGGACAUCCAGGG
UCACAGGUGAGGUUCUUGGGAGCCUGGCGUCUGGCC (hairpin)
or
15-ucccugagacccuuuaaccuguga-38 (mature)
or
53-acaggugagguucuugggagcc-
74 (alternative processing of mature).

By “miR-125a gene” is meant a polynucleotide that encodes a miR-125a microRNA or analog thereof.

By “let-7f-2 microRNA” is meant a nucleic acid molecule comprising a nucleobase sequence that is substantially identical to the sequence of hsa-let-7f-2, MirBase Accession No. MI0000068, MIMAT0000067, or a fragment thereof, whose expression reduces the growth of a neoplasia. The sequence of exemplary let-7f-2 microRNAs follows:

UGUGGGAUGAGGUAGUAGAUUGUAUAGUUUUAGGGUCAUACCCCAUCUUG
GAGAUAACUAUACAGUCUACUGUCUUUCCCACG (hairpin)
or
8-ugagguaguagauuguauaguu-29 (mature).

By “let-7f-2 gene” is meant a polynucleotide that encodes a let-7f-2 microRNA or analog thereof.

By “miR-98 microRNA” is meant a nucleic acid molecule comprising a nucleobase sequence that is substantially identical to the sequence of hsa-mir-98, MirBase Accession No. MI0000100, MIMAT0000096, or a fragment thereof, whose expression reduces the growth of a neoplasia. The sequence of exemplary miR-98 microRNAs follows:

AGGAUUCUGCUCAUGCCAGGGUGAGGUAGUAAGUUGUAUUGUUGUGGGGU
AGGGAUAUUAGGCCCCAAUUAGAAGAUAACUAUACAACUUACUACUUUCC
CUGGUGUGUGGCAUAUUCA (hairpin)
or
22-ugagguaguaaguuguauuguu-43 (mature).

By “miR-98 gene” is meant a polynucleotide that encodes a miR-98 microRNA or analog thereof.

By “let-7g microRNA” is meant a nucleic acid molecule comprising a nucleobase sequence that is substantially identical to the sequence of hsa-let-7g MirBase Accession No. MI0000433, MIMAT0000414, or a fragment thereof, whose expression reduces the growth of a neoplasia. The sequence of exemplary let-7g microRNAs follows:

AGGCUGAGGUAGUAGUUUGUACAGUUUGAGGGUCUAUGAUACCACCCGGU
ACAGGAGAUAACUGUACAGGCCACUGCCUUGCCA. (hairpin),
5-ugagguaguaguuuguacaguu-26 (mature),
or
62-cuguacaggccacugccuugc-82 (minor).

By “let-7g gene” is meant a polynucleotide encoding a let-7g microRNA or analog thereof.

By “let-7i microRNA” is meant a nucleic acid molecule comprising a nucleobase sequence that is substantially identical to the sequence of hsa-let-7i MirBase Accession No. MI0000434, MIMAT0000415, or a fragment thereof whose expression reduces the growth of a neoplasia. The sequence of an exemplary let-7i microRNA follows:

CUGGCUGAGGUAGUAGUUUGUGCUGUUGGUCGGGUUGUGACAUUGCCCGC
UGUGGAGAUAACUGCGCAAGCUACUGCCUUGCUA (hairpin)
or
6-ugagguaguaguuugugcuguu-27.

By “let-7i gene” is meant a polynucleotide that encodes a let-7i microRNA or analog thereof.

By “agent” is meant a polypeptide, polynucleotide, or fragment, or analog thereof, small molecule, or other biologically active molecule.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and most preferably a 50% or greater change in expression levels.

In this disclosure, “comprises,” “comprising,” “containing” and “having” and the like can have the meaning ascribed to them in U.S. Patent law and can mean “ includes,” “including,” and the like; “consisting essentially” of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “control” is meant a standard or reference condition.

By “an effective amount” is meant the amount of an agent required to ameliorate the symptoms of a disease relative to an untreated patient. The effective amount of active agent(s) used to practice the present invention for therapeutic treatment of a neoplasia varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion (e.g., at least 10, 25, 50, 100, 125, 150, 200, 250, 300, 350, 400, or 500 amino acids or nucleic acids) of a protein or nucleic acid molecule that is substantially identical to a reference protein or nucleic acid and retains the biological activity of the reference protein or nucleic acid.

A “host cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

By “inhibits a neoplasia” is meant decreases the propensity of a cell to develop into a neoplasia or slows, decreases, or stabilizes the growth or proliferation of a neoplasia.

By “isolated nucleic acid molecule” is meant a nucleic acid (e.g., a DNA, RNA, microRNA or analog thereof) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes a microRNA or other RNA molecule which is transcribed from a DNA molecule, as well as a recombinant DNA which is part of a hybrid gene encoding additional polypeptide sequence.

By “marker” is meant any protein or polynucleotide having an alteration in expression level or activity that is associated with a disease or disorder.

The term “microarray” is meant to include a collection of nucleic acid molecules or polypeptides from one or more organisms arranged on a solid support (for example, a chip, plate, or bead).

By “modification” is meant any biochemical or other synthetic alteration of a nucleotide, amino acid, or other agent relative to a naturally occurring reference agent.

By “neoplasia” is meant any disease that is caused by or results in inappropriately high levels of cell division, inappropriately low levels of apoptosis, or both. For example, cancer is a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “mature form” is meant a microRNA that has, at least in part, been processed into a biologically active form that can participate in the regulation of a target mRNA.

By “hairpin form” is meant a microRNA that includes a double stranded portion.

By “microRNA” is meant a nucleobase sequence having biological activity that is independent of any polypeptide encoding activity. MicroRNAs may be synthetic or naturally occurring, and may include one or more modifications described herein. MicroRNAs include pri-microRNAs, hairpin microRNAs, and mature microRNAs.

By “Myc disregulation” is meant an alteration in the level of expression of one or more microRNAs usually repressed by Myc.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

By “obtaining” as in “obtaining the inhibitory nucleic acid molecule” is meant synthesizing, purchasing, or otherwise acquiring the inhibitory nucleic acid molecule.

By “oligonucleotide” is meant any molecule comprising a nucleobase sequence. An oligonucleotide may, for example, include one or more modified bases, linkages, sugar moieties, or other modifications.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant microRNA molecule described herein).

“Primer set” or “probe set” means a set of oligonucleotides. A primer set may be used, for example, for the amplification of a polynucleotide of interest. A probe set may be used, for example, to hybridize with a polynucleotide of interest. A primer set would consist of at least 2, 4, 6, 8, 10, 12, 14, 16, 18, 20, 30, 40, 50, 60, 80, 100, or more primers or probes.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 6, 7, 8, 9, 10, 11, 12, 13, 14, 15, 16, 17, 18, 19, 20, or 21 nucleotides.

By “reduces” is meant a negative alteration. A reduction includes, for example, a 5%, 10%, 25%, 50%, 75% or even 100% reduction.

By “reduces the survival” is meant increases the probability of cell death in a cell or population of cells relative to a reference. For example, a reduction in survival is measured in a cell treated with a microRNA of the invention relative to an untreated control cell. Cell death may be by any means, including apoptotic or necrotic cell death.

By “reduces cell division” is meant interferes with the cell cycle or otherwise reduces the growth or proliferation of a cell, tissue, or organ relative to a reference. For example, a reduction in cell division is measured in a cell treated with a microRNA of the invention relative to an untreated control cell.

By “reference” is meant a standard or control condition.

By “reporter gene” is meant a gene encoding a polypeptide whose expression may be assayed; such polypeptides include, without limitation, glucuronidase (GUS), luciferase, chloramphenicol transacetylase (CAT), and beta-galactosidase.

The term “subject” is intended to include vertebrates, preferably a mammal. Mammals include, but are not limited to, humans.

The term “pharmaceutically-acceptable excipient” as used herein means one or more compatible solid or liquid filler, diluents or encapsulating substances that are suitable for administration into a human.

By “transformed cell” is meant a cell into which (or into an ancestor of which) has been introduced, by means of recombinant DNA techniques, a polynucleotide molecule encoding (as used herein) a protein of the invention.

By “vector” is meant a nucleic acid molecule, for example, a plasmid, cosmid, or bacteriophage, that is capable of replication in a host cell. In one embodiment, a vector is an expression vector that is a nucleic acid construct, generated recombinantly or synthetically, bearing a series of specified nucleic acid elements that enable transcription of a nucleic acid molecule in a host cell. Typically, expression is placed under the control of certain regulatory elements, including constitutive or inducible promoters, tissue-preferred regulatory elements, and enhancers.

In one embodiment, nucleic acid molecules useful in the methods of the invention include any nucleic acid molecule that encodes a polypeptide of the invention or a fragment thereof. In another embodiment, nucleic acid molecules useful in the methods of the invention include any nucleic acid molecule that encodes a polynucleotide (e.g., a microRNA) that has biologic activity independent of providing a polypeptide sequence. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42.degree. C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least. 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

### BRIEF DESCRIPTION OF THE DRAWINGS

FIGS. 1A-1D show repression of miRNA expression by Myc. FIG. 1A shows the results of a Northern blot analysis of miRNAs in P493-6 cells with high Myc or low Myc expression. U6 snRNA served as a loading control for this and all subsequent experiments (representative blot shown). ‘Expression ratio’ in this and subsequent figures indicates the expression level of the miRNA in the high Myc state relative to the low Myc state. “ND” denotes not detectable. FIG. 1B is a table showing the organization of the human miR-30 clusters. miRNA clusters downregulated by Myc, as determined in c, are shown in bold. FIG. 1C shows the results for Northern blots demonstrating repression of miR-30 family members by Myc. Synthetic RNA oligonucleotides identical in sequence to each miR-30 family member and total RNA from P493-6 cells were hybridized with probes specific for each miRNA. FIG. 1D shows repression of miRNAs in MycER tumors. FIG. 1D shows the results of a Northern blot analysis of miRNAs in MycER tumors. ‘Expression Ratio’ indicates the level of miRNA expression in the MycON state relative to the MycOFF state. Specific hybridization conditions, as shown in FIGS. 1C and 4B, were used for miR-30b and let-7a. tRNALys served as a loading control (representative blot shown).

FIGS. 2A-2C show that Myc represses miRNAs in Burkitt's lymphoma cells. FIG. 2A shows an analysis of previously published miRNA expression profiling data (He et al., Nature, 2005), which demonstrates that most Myc repressed miRNAs are expressed at lower levels in Burkitt's lymphoma cells compared to normal B cells. FIG. 2B provides the results of a Western blot showing Myc knockdown by lentivirally-expressed shRNA in EW36 Burkitt's lymphoma cells. shRNA directed against luciferase (Luc) served as a negative control. FIG. 2C shows that Myc knockdown results in upregulation of miRNAs in EW36 cells. miR-29a was not upregulated by Myc shRNA under these conditions and miR-34a and miR-150 were not expressed at detectable levels in this cell line (not shown).

FIGS. 3A-3B show that Myc associates with repressed pri-miRNA promoters. FIG. 3A provides schematic representations of repressed pri-miRNAs of known structure. FIG. 3B shows that real-time PCR amplicons for ChIP were designed within 250 bp windows immediately upstream of the transcription start site (amplicon S), 500 bp upstream of amplicon S (amplicon U), or 500 bp downstream of amplicon S (amplicon D). FIG. 3C is a graph showing the results of a real-time PCR analysis of Myc chromatin immunoprecipitates. Fold enrichment for this and subsequent ChIP experiments represents signal obtained following Myc immunoprecipitation relative to signal obtained with irrelevant antibody. A validated Myc-bound amplicon in the promoter region of CDKN1A (p21^{WAF1/CIP1}) served as a positive control. The 50-fold enrichment threshold for positive Myc binding is indicated as a dashed line. Error bars represent standard deviations derived from three independent measurements.

FIGS. 4A-4C show that Myc associates with conserved regions upstream of repressed miRNAs. FIG. 4A illustrates the phylogenetic conservation of the intergenic region containing the miR-29b-2/29c cluster. VISTA was used to generate pairwise alignments between genomic sequence from human (May 2004 assembly) and the species listed on the left. The graph is a plot of nucleotide identity for a 100 base-pair sliding window centered at a given position. Annotated transcripts produced from this locus are shown at the top of the panel. Note that the 5′ end of miR-29b-2/29c is towards the right. Locations of real-time PCR amplicons used for ChIP experiments are indicated as arrows below the graph. “C” denotes conserved amplicon; “N” denotes a negative control amplicon. FIG. 4B is a graph showing the results of the Real-time PCR analysis of Myc chromatin immunoprecipitates as described in FIG. 3C. The conserved amplicon that exhibited maximal Myc binding (C) and a representative negative control amplicon (N) are shown for each miRNA. Locations of these and additional amplicons for the miR-29b-1/29a cluster, the miR-30d/30b cluster, miR-34a, miR-146a, the miR-195/497 cluster, and miR-150 are shown in FIGS. 5-8. (c) Conserved Myc binding sites correspond to pri-miRNA promoters. The structure of pri-miRNA transcripts as defined by 5′ and 3′ RACE are depicted. In some cases, alternative splicing was observed giving rise to major and minor transcript isoforms. Plots representing evolutionary conservation, below each transcript, were taken from the UCSC genome browser (human genome May 2004 assembly). The locations of ChIP amplicons that yielded highest Myc binding signals are indicated with arrows.

FIGS. 5A-5B shows that Myc associates with a conserved region upstream of the miR-29b-1/29a cluster. FIG. 5A shows a VISTA analysis of phylogenetic conservation encompassing the miR-29b-1/29a cluster as described in FIG. 4A. Amplicons shown in FIG. 4B are bolded and underlined. FIG. 5B shows a Real-time PCR analysis of Myc chromatin immunoprecipitates as described in FIG. 3C.

FIGS. 6A and 6B shows that Myc associates with a conserved region upstream of the miR-30d/30b cluster. FIG. 6A shows a VISTA analysis of phylogenetic conservation encompassing the miR-30d/30b cluster as described in FIG. 4A. Amplicons shown in FIG. 4B are bolded and underlined. FIG. 6B shows a real-time PCR analysis of Myc chromatin immunoprecipitates as described in FIG. 3C.

FIGS. 7A and 7B show that Myc associates with a conserved region upstream of miR-34a. FIG. 7A shows a VISTA analysis of phylogenetic conservation encompassing miR-34a as described in FIG. 4a. Amplicons shown in FIG. 4B are bolded and underlined. FIG. 7B shows a real-time PCR analysis of Myc chromatin immunoprecipitates as described in FIG. 3C.

FIGS. 8A and 8B show that Myc associates with a conserved region upstream of miR-146a. FIG. 8A shows a VISTA analysis of phylogenetic conservation encompassing miR-146a as described in FIG. 4A. Amplicons shown in FIG. 4B are bolded and underlined. FIG. 8B shows a real-time PCR analysis of Myc chromatin immunoprecipitates as described in FIG. 3C.

FIGS. 9A and 9B show that Myc associates with a conserved region upstream of the miR-195/497 cluster. FIG. 9A shows a VISTA analysis of phylogenetic conservation encompassing the miR-195/497 cluster as described in FIG. 4A. Amplicons shown in FIG. 4B are bolded and underlined. FIG. 9B shows a Real-time PCR analysis of Myc chromatin immunoprecipitates as described in FIG. 3C.

FIGS. 10A and 10B show that Myc does not associate with conserved regions upstream of miR-150. FIG. 10A shows a VISTA analysis of phylogenetic conservation encompassing miR-150 as described in FIG. 3a. Amplicons shown in FIG. 4B are bolded and underlined. FIG. 10B shows a real-time PCR analysis of Myc chromatin immunoprecipitates as described in FIG. 3C.

FIGS. 11A and 11B show that Myc does not associate with conserved regions upstream of the miR-30a/30c-2 cluster. FIG. 11A shows a VISTA analysis of phylogenetic conservation encompassing the miR-30a/30c-2 cluster as described in FIG. 3A. FIG. 11B shows a real-time PCR analysis of Myc chromatin immunoprecipitates as described in FIG. 3C.

FIGS. 12A-12D show that let-7 miRNAs are downregulated by Myc. FIG. 12A shows the organization of the human let-7 clusters. miRNA clusters downregulated by Myc, as determined in FIGS. 12B-D, are shown in bold. Northern blot analysis of synthetic RNA oligonucleotides or total RNA from P493-6 cells was performed with probes specific for each member of the let-7 family. FIGS. 12B and 12C show results for the miR-99/100 family. FIG. 12D shows results for the miR-125 family. “ND” denotes not detectable.

FIGS. 13A and 13B show that Myc binds to conserved regions upstream of let-7 miRNAs. FIG. 13A shows a VISTA analysis of phylogenetic conservation encompassing the let-7a-1/let-7f-1/let-7d cluster, let-7g, and the miR-99a/let-7c/miR-125b-2 cluster as described in FIG. 4A. FIG. 13B shows a real-time PCR analysis of Myc chromatin immunoprecipitates as described in FIG. 3C.

FIGS. 14A and 14B show that expression of Myc-repressed miRNAs disadvantages lymphoma cell growth in vivo. FIG. 14A is a schematic diagram illustrating the infection of Myc3 or 38B9 lymphoma cells with a retrovirus that expresses a miRNA and GFP. The fraction of GFP positive cells was measured before and after tumor formation. FIG. 14B is a graph showing that cells expressing select miRNAs are eliminated from tumors. Standard deviations of measurements from three independent trials are shown. All cultures were at least 30% GFP positive prior to injection into recipient mice.

FIGS. 15A and 15B are Northern blots showing retroviral miRNA expression levels in Myc3 and 38B9 cells. Numbers below blots represent the expression level of each miRNA relative to the non-transformed B cell line YSPB11. All quantifications were normalized to to loading control (tRNALys, not shown) and to P493 (low Myc) RNA which was loaded on each gel to allow direct comparison of miRNA levels across blots. In FIG. 15B retroviral miR-150 expression was compared to MycOFF tumors since this miRNA was not expressed in YS-PB11 cells.

FIGS. 16A and 16B show the kinetics of miRNA repression following Myc-induction in P493-6 cells. FIG. 16A shows results of a Western blot demonstrating Myc induction following removal of tetracycline (tet). Leftmost tet (+) or tet (−) lanes represent cells grown with or without tet for 72 hours. FIG. 16B shows the results of Northern blots demonstrating miRNA repression following tet release. Numbers below blots represent expression level of each miRNA relative to tet (+) level, normalized to loading control (tRNALys, not shown). Under these conditions, P493-6 cells do not begin proliferating until 48 hours after tet removal and do not reach maximal growth rates until at least 72 hours after tet removal (our unpublished observations and O'Donnell et al., Mol Cell Bio, 2006).

FIGS. 17A-17D shows sequences of microRNAs described herein. FIG. 17A corresponds to microRNA 29b-1/29a, microRNA 29b-1, and microRNA 29a genes (GenBank Accession No. EU154353). FIG. 17B shows Homo sapiens microRNA 29b-2/29c, precursor RNA, microRNA 29b-2 and microRNA 29c, (GenBank Accession Nos. EU154351). FIG. 17C provides the sequence of microRNA 29b-2/29c, precursor RNA, microRNA 29b-2 and microRNA 29c (GenBank Accession No. EU154352). FIG. 17D provides the sequence of miR-146a (GenBank Accession No. EU147785).

### DETAILED DESCRIPTION OF THE INVENTION

The invention provides compositions and methods featuring microRNAs that are useful for treating or preventing a neoplasia. Myc directly activates transcription of the mir-17 cluster (O'Donnell et al., Nature 435, 839-43 (2005)). To identify Myc-regulated miRNAS an analysis of human and mouse models of Myc-mediated lymphomagenesis was undertaken. This analysis led to the discovery of a large set of Myc-regulated miRNAs. Remarkably, induction of Myc resulted primarily in widespread downregulation of miRNA expression. Chromatin immunoprecipitation (ChIP) revealed that Myc binds directly to promoters or conserved regions upstream of the miRNAs that it represses. The invention is based, at least in part, on the discovery that the expression of Myc-repressed miRNAs dramatically impeded lymphoma cell growth in vivo. These observations indicate that repression of tumor-suppressing miRNAs is a fundamental component of the Myc tumorigenic program. Accordingly, the invention provides compositions and methods featuring miRNAs whose expression is useful for the treatment or prevention of neoplasia.

As reported in more detail below, Myc repressed expression of the following microRNAs by at least about 2-fold: miR-22, miR-26a-1, miR-26a-2, miR-29b-2, miR-29c, miR-30e, miR-30c-1, miR-146a, miR-150. Myc repressed expression of let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-15a, miR-16-1, miR-29b-1, miR-29a, miR-34a, miR-195, miR-26b, and miR-30c by at least about 1.5 fold in two models of neoplasia. Therefore, the expression of one or more of these Myc-repressed microRNAs or a fragment thereof, is expected to be useful for the treatment or prevention of a neoplasia.

Significantly, when miR-34a, miR-150, miR-195/497, and miR-15a/16-1 were expressed in neoplastic cells within tumors, cells expressing these microRNAs were virtually eliminated from the tumors. This indicates that these miRNAs possess anti-tumorigenic properties in the setting of both Myc- and v-Abl-mediated transformation. miR-26a suppressed tumorigenesis in the setting of Myc-mediated transformation and miR-22 suppressed tumorigenesis in the setting of v-Abl-mediated transformation. In view of these findings, agents that increase the expression of a microRNA described herein within a neoplastic cell are expected to be useful for the treatment or prevention of a variety of neoplasias.

### MicroRNAs

MicroRNAs are small noncoding RNA molecules that are capable of causing post-transcriptional silencing of specific genes in cells by the inhibition of translation or through degradation of the targeted mRNA. A microRNA can be completely complementary or can have a region of noncomplementarity with a target nucleic acid, consequently resulting in a “bulge” at the region of non-complementarity. A microRNA can inhibit gene expression by repressing translation, such as when the microRNA is not completely complementary to the target nucleic acid, or by causing target RNA degradation, which is believed to occur only when the microRNA binds its target with perfect complementarity. The invention also can include double-stranded precursors of microRNA.

A microRNA or pre-microRNA can be 18-100 nucleotides in length, and more preferably from 18-80 nucleotides in length. Mature miRNAs can have a length of 19-30 nucleotides, preferably 21-25 nucleotides, particularly 21, 22, 23, 24, or 25 nucleotides. MicroRNA precursors typically have a length of about 70-100 nucleotides and have a hairpin conformation. MicroRNAs are generated in vivo from pre-miRNAs by the enzymes Dicer and Drosha, which specifically process long pre-miRNA into functional miRNA. The hairpin or mature microRNAs, or pre-microRNA agents featured in the invention can be synthesized in vivo by a cell-based system or in vitro by chemical synthesis.

The invention provides isolated microRNAs and polynucleotides encoding such sequences. A recombinant microRNA of the invention (e.g., miR-22, miR-26a-1, miR-26a-2, mir-26b, mir-29b-1, mir-29a, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1) or a polynucleotide encoding such a microRNA may be administered to reduce the growth, survival, or proliferation of a neoplastic cell in a subject in need thereof. In one approach, the microRNA is administered as a naked RNA molecule. In another approach, it is administered in an expression vector suitable for expression in a mammalian cell.

One exemplary approach provided by the invention involves administration of a recombinant therapeutic, such as a recombinant microRNA molecule, variant, or fragment thereof, either directly to the site of a potential or actual disease-affected tissue or systemically (for example, by any conventional recombinant administration technique). The dosage of the administered microRNA depends on a number of factors, including the size and health of the individual patient. For any particular subject, the specific dosage regimes should be adjusted over time according to the individual need and the professional judgment of the person administering or supervising the administration of the compositions.

For example, a microRNA of the invention (e.g., (e.g., miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1) may be administered in dosages between about 1 and 100 mg/kg (e.g., 1, 5, 10, 20, 25, 50, 75, and 100 mg/kg). In other embodiments, the dosage ranges from between about 25 and 500 mg/m²/day. Desirably, a human patient having a neoplasia receives a dosage between about 50 and 300 mg/m²/day (e.g., 50, 75, 100, 125, 150, 175, 200, 250, 275, and 300).

MicroRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism. Modifications can also increase sequence specificity, and consequently decrease off-site targeting. Methods of synthesis and chemical modifications are described in greater detail below.

The invention further provides solid supports, including microarrays, comprising one, two, three, four, five, six or more microRNAs, oligonucleotides comprising such microRNAs, or nucleic acid sequences encoding or binding to such microRNAs. In addition, the invention provides probes that hybridize to and/or that may be used to amplify a microRNA of the invention. In particular embodiments, the invention provides collections of such probes that include one, two, three, four, or more microRNAs or probes described herein.

### MicroRNA Analogs

If desired, microRNA molecules may be modified to stabilize the microRNAs against degradation, to enhance half-life, or to otherwise improve efficacy. Desirable modifications are described, for example, in U.S. Patent Publication Nos. 20070213292, 20060287260, 20060035254, 20060008822, and 20050288244, each of which is hereby incorporated by reference in its entirety.

For increased nuclease resistance and/or binding affinity to the target, the single-stranded oligonucleotide agents featured in the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications can also increase binding affinity to the target. The inclusion of pyranose sugars in the oligonucleotide backbone can also decrease endonucleolytic cleavage. An antagomir can be further modified by including a 3′ cationic group, or by inverting the nucleoside at the 3′-terminus with a 3′-3′ linkage. In another alternative, the 3′-terminus can be blocked with an aminoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In one embodiment, the microRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed to enhance the stability or efficacy of an inhibitory nucleic acid molecule.

MicroRNA molecules include nucleobase oligomers containing modified backbones or non-natural internucleoside linkages. Oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers. Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Nucleobase oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleobase oligomer, particularly the 3′ position of the sugar on the 3′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In other nucleobase oligomers, both the sugar and the internucleoside linkage, i.e., the backbone, are replaced with novel groups. The nucleobase units are maintained for hybridization with a nucleic acid molecule of the miR-17-92 cluster. Methods for making and using these nucleobase oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In other embodiments, a single stranded modified nucleic acid molecule (e.g., a nucleic acid molecule comprising a phosphorothioate backbone and 2′-O-Me sugar modifications is conjugated to cholesterol.

### Delivery of Nucleobase Oligomers

A microRNA of the invention, which may be in the mature or hairpin form, may be provided as a naked oligonucleotide that is capable of entering a tumor cell. In some cases, it may be desirable to utilize a formulation that aids in the delivery of a microRNA or other nucleobase oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the microRNA composition is at least partially crystalline, uniformly crystalline, and/or anhydrous (e.g., less than 80, 50, 30, 20, or 10% water). In another example, the microRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the microRNA composition is formulated in a manner that is compatible with the intended method of administration.

A microRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg²⁺), salts, and RNAse inhibitors (e.g., a broad specificity RNAse inhibitor, such as RNAsin).

In one embodiment, the microRNA composition includes another microRNA, e.g., a second microRNA composition (e.g., a microRNA that is distinct from the first). Still other preparations can include at least three, five, ten, twenty, fifty, or a hundred or more different oligonucleotide species.

### Polynucleotide Therapy

Polynucleotide therapy featuring a polynucleotide encoding a microRNA is another therapeutic approach for inhibiting neoplasia in a subject. Expression vectors encoding the microRNAs can be delivered to cells of a subject for the treatment or prevention of a neoplasia. The nucleic acid molecules must be delivered to the cells of a subject in a form in which they can be taken up and are advantageously expressed so that therapeutically effective levels can be achieved.

Methods for delivery of the polynucleotides to the cell according to the invention include using a delivery system, such as liposomes, polymers, microspheres, gene therapy vectors, and naked DNA vectors.

Transducing viral (e.g., retroviral, adenoviral, lentiviral and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a microRNA molecule can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No.5,399,346).

Non-viral approaches can also be employed for the introduction of a microRNA therapeutic to a cell of a patient diagnosed as having a neoplasia. For example, a microRNA can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the microRNA molecules are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell.

Microrna expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers.

For any particular subject, the specific dosage regimes should be adjusted over time according to the individual need and the professional judgment of the person administering or supervising the administration of the compositions.

### Pharmaceutical Compositions

As reported herein, a reduction in the expression of specific microRNAs regulated by Myc is associated with neoplasia or tumorigenesis. Accordingly, the invention provides therapeutic compositions that increase the expression of a microRNAs described herein for the treatment or prevention of a neoplasm. In one embodiment, the present invention provides a pharmaceutical composition comprising a microRNA of the invention or a nucleic acid molecule encoding a microRNA of the invention. If desired, the nucleic acid molecule is administered in combination with a chemotherapeutic agent. In another embodiment, a recombinant microRNA or a polynucleotide encoding such a microRNA, is administered to reduce the growth, survival or proliferation of a neoplastic cell or to increase apoptosis of a neoplastic cell. Polynucleotides of the invention may be administered as part of a pharmaceutical composition. The compositions should be sterile and contain a therapeutically effective amount of a microRNA or nucleic acid molecule encoding a microRNA in a unit of weight or volume suitable for administration to a subject.

A recombinant microRNA or a nucleic acid molecule encoding a microRNA described herein may be administered within a pharmaceutically-acceptable diluent, carrier, or excipient, in unit dosage form. Conventional pharmaceutical practice may be employed to provide suitable formulations or compositions to administer the compounds to patients suffering from a neoplasia. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for inhibitory nucleic acid molecules include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a neoplastic disease or condition. The preferred dosage of a nucleobase oligomer of the invention is likely to depend on such variables as the type and extent of the disorder, the overall health status of the particular patient, the formulation of the compound excipients, and its route of administration.

With respect to a subject having a neoplastic disease or disorder, an effective amount is sufficient to stabilize, slow, or reduce the proliferation of the neoplasm. Generally, doses of active polynucleotide compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels a microRNA of the invention or of a polynucleotide encoding such a microRNA.

Accordingly, the present invention provides methods of treating disease and/or disorders or symptoms thereof which comprise administering a therapeutically effective amount of a composition comprising a microRNA described herein to a subject (e.g., a mammal, such as a human). Thus, one embodiment is a method of treating a subject suffering from or susceptible to a neoplastic disease or disorder or symptom thereof. The method includes the step of administering to the mammal a therapeutic amount of a microRNA or nucleic acid encoding such a microRNA herein sufficient to treat the neoplastic disease or disorder or symptom thereof, under conditions such that the disease or disorder is treated.

The methods herein include administering to the subject (including a subject identified as in need of such treatment) an effective amount of a compound described herein, or a composition described herein to prevent, treat, stabilize, or reduce the growth or survival of a neoplasia in a subject in need thereof. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the agents herein, such as a microRNA or a nucleic acid encoding such a microRNA herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (e.g., increased Myc expression or a neoplasia associated with an alteration in Myc regulation, or as defined herein), family history, and the like). The compounds herein may be also used in the treatment of any other disorders in which Myc dysregulation may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with Myc disregulation, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Therapy

Therapy may be provided wherever cancer therapy is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of neoplasia being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of cancer and its stage of development, the therapy can be used to slow the spreading of the cancer, to slow the cancer's growth, to kill or arrest cancer cells that may have spread to other parts of the body from the original tumor, to relieve symptoms caused by the cancer, or to prevent cancer in the first place. As described above, if desired, treatment with a microRNA or a polynucleotide encoding such a microRNA may be combined with therapies for the treatment of proliferative disease (e.g., radiotherapy, surgery, or chemotherapy). For any of the methods of application described above, microRNA of the invention is desirably administered intravenously or is applied to the site of neoplasia (e.g., by injection).

### Diagnostics

As described in more detail below, the present invention has identified reductions in the expression of Myc regulated microRNAs (e.g., miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1) that are associated with neoplasia. Reductions in the expression level of one or more of these markers is used to diagnose a subject as having a neoplasia associated with Myc disregulation. In one embodiment, the method identifies a neoplasia as amenable to treatment using a method of the invention by assaying a decrease in the level of any one or more of the following markers: miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1.

In one embodiment, a subject is diagnosed as having or having a propensity to develop a neoplasia, the method comprising measuring markers in a biological sample from a patient, and detecting an alteration in the expression of one or more marker molecules relative to the sequence or expression of a reference molecule. The markers typically include a microRNA.

Reduced expression of a microRNA of the invention (e.g., miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1) is used to identify a neoplasia that is amenable to treatment using a composition or method described herein. Accordingly, the invention provides compositions and methods for identifying such neoplasias in a subject. Alterations in gene expression are detected using methods known to the skilled artisan and described herein. Such information can be used to diagnose a neoplasia or to identify a neoplasia as being amenable to a therapeutic method of the invention.

In one approach, diagnostic methods of the invention are used to assay the expression of a microRNA (e.g., miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1) in a biological sample relative to a reference (e.g., the level of microRNA present in a corresponding control tissue, such as a healthy tissue). Exemplary nucleic acid probes that specifically bind a microRNA of the invention are described herein. By “nucleic acid probe” is meant any nucleic acid molecule, or fragment thereof, that binds or amplifies a microRNA of the invention. Such nucleic acid probes are useful for the diagnosis of a neoplasia.

In one approach, quantitative PCR methods are used to identify a reduction in the expression of a microRNA of the invention. In another approach, a probe that hybridizes to a microRNA of the invention is used. The specificity of the probe determines whether the probe hybridizes to a naturally occurring sequence, allelic variants, or other related sequences. Hybridization techniques may be used to identify mutations indicative of a neoplasia or may be used to monitor expression levels of these genes (for example, by Northern analysis (Ausubel et al., supra).

In general, the measurement of a nucleic acid molecule or a protein in a subject sample is compared with a diagnostic amount present in a reference. A diagnostic amount distinguishes between a neoplastic tissue and a control tissue. The skilled artisan appreciates that the particular diagnostic amount used can be adjusted to increase sensitivity or specificity of the diagnostic assay depending on the preference of the diagnostician. In general, any significant increase or decrease (e.g., at least about 10%, 15%, 30%, 50%, 60%, 75%, 80%, or 90%) in the level of test nucleic acid molecule or polypeptide in the subject sample relative to a reference may be used to diagnose or characterize a neoplasia. Test molecules include any one or more of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1. In one embodiment, the reference is the level of test polypeptide or nucleic acid molecule present in a control sample obtained from a patient that does not have a neoplasia. In another embodiment, the reference is a baseline level of test molecule present in a biologic sample derived from a patient prior to, during, or after treatment for a neoplasia. In yet another embodiment, the reference can be a standardized curve.

### Types of Biological Samples

The level of markers in a biological sample from a patient having or at risk for developing a neoplasia can be measured, and an alteration in the expression of marker molecule relative to the sequence or expression of a reference molecule, can be determined in different types of biologic samples. Test markers include any one or all of the following: miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1. The biological samples are generally derived from a patient, preferably as a bodily fluid (such as blood, cerebrospinal fluid, phlegm, saliva, or urine) or tissue sample (e.g. a tissue sample obtained by biopsy).

### Kits

The invention provides kits for the prevention, treatment, diagnosis or monitoring of a neoplasia. In one embodiment, the kit provides a microRNA molecule for administration to a subject. In another embodiment, the kit detects an alteration in the sequence or expression of a miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1 derived from a subject relative to a reference sequence or reference level of expression. In related embodiments, the kit includes reagents for monitoring the expression of a miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a116-1 nucleic acid molecule, such as primers or probes that hybridize to a miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1 nucleic acid molecule.

Optionally, the kit includes directions for monitoring the nucleic acid molecule levels of a Marker in a biological sample derived from a subject. In other embodiments, the kit comprises a sterile container which contains the primer, probe, antibody, or other detection regents; such containers can be boxes, ampoules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in diagnosing a neoplasia. Preferably, the kit further comprises any one or more of the reagents described in the diagnostic assays described herein. In other embodiments, the instructions include at least one of the following: description of the primer or probe; methods for using the enclosed materials for the diagnosis of a neoplasia; precautions; warnings; indications; clinical or research studies; and/or references. The instructions may be printed directly on the container (when present), or as a label applied to the container, or as a separate sheet, pamphlet, card, or folder supplied in or with the container.

### Screening Assays

One embodiment of the invention encompasses a method of identifying an agent that increases the expression or activity of a miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, or miR-15a/16-1 microRNA. Accordingly, compounds that increase the expression or activity of a microRNA of the invention or a variant, or portion thereof are useful in the methods of the invention for the treatment or prevention of a neoplasm. The method of the invention may measure an increase in transcription of one or more microRNAs of the invention. Any number of methods are available for carrying out screening assays to identify such compounds. In one approach, the method comprises contacting a cell that expresses a microRNA of the invention (e.g., miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1) with an agent and comparing the level of expression in the cell contacted by the agent with the level of expression in a control cell, wherein an agent that increases the expression of a microRNA of the invention thereby inhibits a neoplasia.

In other embodiments, the agent acts as a microRNA mimetic, which substantially fulfills the function of an microRNA of the invention. Candidate mimetics include organic molecules, peptides, polypeptides, nucleic acid molecules. Small molecules of the invention preferably have a molecular weight below 2,000 daltons, more preferably between 300 and 1,000 daltons, and still more preferably between 400 and 700 daltons. It is preferred that these small molecules are organic molecules. Compounds isolated by any approach described herein may be used as therapeutics to treat a neoplasia in a human patient.

In addition, compounds that increase the expression of a microRNA of the invention are also useful in the methods of the invention. Any number of methods are available for carrying out screening assays to identify new candidate compounds that increase the expression of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, or miR-15a/16-1. The invention also includes novel compounds identified by the above-described screening assays. Optionally, such compounds are characterized in one or more appropriate animal models to determine the efficacy of the compound for the treatment of a neoplasia. Desirably, characterization in an animal model can also be used to determine the toxicity, side effects, or mechanism of action of treatment with such a compound. Furthermore, novel compounds identified in any of the above-described screening assays may be used for the treatment of a neoplasia in a subject. Such compounds are useful alone or in combination with other conventional therapies known in the art.

### Test Compounds and Extracts

In general, compounds capable of inhibiting the growth or proliferation of a neoplasia by increasing the expression or biological activity of a microRNA (e.g., miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1) are identified from large libraries of either natural product or synthetic (or semi-synthetic) extracts or chemical libraries according to methods known in the art. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.).

In one embodiment, test compounds of the invention are present in any combinatorial library known in the art, including: biological libraries; peptide libraries (libraries of molecules having the functionalities of peptides, but with a novel, non-peptide backbone which are resistant to enzymatic degradation but which nevertheless remain bioactive; see, e.g., Zuckermann, R. N. et al., J. Med. Chem. 37:2678-85, 1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des. 12:145, 1997).

Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-neoplastic activity should be employed whenever possible.

In an embodiment of the invention, a high thoroughput approach can be used to screen different chemicals for their potency to enhance the activity of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, or miR-15a/16-1.

Those skilled in the field of drug discovery and development will understand that the precise source of a compound or test extract is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

When a crude extract is found to enhance the biological activity of a miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1, variant, or fragment thereof, further fractionation of the positive lead extract is necessary to isolate chemical constituents responsible for the observed effect. Thus, the goal of the extraction, fractionation, and purification process is the careful characterization and identification of a chemical entity within the crude extract having anti-neoplastic activity. Methods of fractionation and purification of such heterogeneous extracts are known in the art. If desired, compounds shown to be useful agents for the treatment of a neoplasm are chemically modified according to methods known in the art.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Examples

### Example 1

### Identification of Myc-Repressed miRNAs

A spotted oligonucleotide array was used to identify the mir-17 cluster as a direct transcriptional target of Myc (O'Donnell et al., Nature 435, 839-43 (2005)). In order to determine whether Myc regulates additional miRNAs, custom microarrays were produced with an expanded set of probes capable of assaying the expression of 313 human miRNAs and 233 mouse miRNAs. Two models of Myc-mediated tumorigenesis were chosen for analysis. P493-6 cells, which are Epstein-Barr virus-immortalized human B cells that harbor a tetracycline (tet)-repressible allele of Myc (Pajic et al., Int J Cancer 87, 787-93 (2000)) were used. These cells are tumorigenic in immunocompromised mice and represent a model of human B cell lymphoma (Gao et al., Cancer Cell 12, 230-8 (2007)). miRNA expression profiles were examined in the high Myc (−tet) and low Myc (+tet) state. miRNA expression was also assayed in a murine model of Myc-induced B cell lymphoma. In this system, bone marrow from p53^{-/-} mice was infected with a retrovirus that produces a Myc-estrogen receptor fusion protein (MycER). Infected cells form polyclonal B cell lymphomas in the presence of 4-hydroxytamoxifen (4-OHT), which activates the MycER fusion protein (Yu et al., Cancer Research 65, 5454-5461 (2005), Yu et al., Oncogene 21, 1922-7 (2002)). RNA from subcutaneous tumors with high Myc activity (animals treated continuously with 4-OHT) and low Myc activity (animals in which 4-OHT was withdrawn after tumor formation) was analyzed. Complete expression profiling data for both models is provided in Tables 1 and 2 (below).

TABLE 1
Expression Profile

| | | | fold change high/ |
|---|---|---|---|
| Name | P493 low Myc | P493 high Myc | low Myc |
| hsa\|miR-128b\|as\| | 0 | 526 | #DIV/0! |
| hsa\|miR-213\|as\| | 0 | 577 | #DIV/0! |
| hsa\|miR-7\|as\| | 0 | 724 | #DIV/0! |
| hsa\|miR-19a\|as\| | 2275 | 5844 | 2.5686 |
| hsa\|miR-17-3p\|as\| | 1068 | 2576 | 2.4118 |
| hsa\|miR-106a\|as\| | 22755 | 54441 | 2.3925 |
| hsa\|miR-17-5p\|as\| | 21034 | 47917 | 2.2781 |
| hsa\|miR-20a\|as\| | 21685 | 47513 | 2.1910 |
| hsa\|miR-20b\|as\| | 13081 | 27789 | 2.1245 |
| hsa\|miR-92\|as\| | 1801 | 3704 | 2.0569 |
| hsa\|miR-422a\|as\| | 879 | 1769 | 2.0136 |
| hsa\|miR-19b\|as\| | 12994 | 24676 | 1.8990 |
| hsa\|miR-18a\|as\| | 2903 | 5347 | 1.8423 |
| hsa\|miR-18b\|as\| | 2083 | 3356 | 1.6118 |
| hsa\|miR-422b\|as\| | 2260 | 3493 | 1.5459 |
| hsa\|miR-324-5p\|as\| | 804 | 1127 | 1.4010 |
| hsa\|miR-301\|as\| | 479 | 661 | 1.3798 |
| hsa\|miR-106b\|as\| | 5224 | 7177 | 1.3739 |
| hsa\|miR-101\|as\| | 1633 | 2238 | 1.3703 |
| hsa\|miR-93\|as\| | 3499 | 4620 | 1.3202 |
| hsa\|miR-185\|as\| | 589 | 727 | 1.2346 |
| hsa\|miR-188\|as\| | 839 | 1021 | 1.2178 |
| hsa\|miR-345\|as\| | 416 | 505 | 1.2135 |
| hsa\|miR-320\|as\| | 1205 | 1453 | 1.2056 |
| hsa\|miR-25\|as\| | 2362 | 2807 | 1.1884 |
| hsa\|miR-199a*\|as\| | 858 | 1010 | 1.1772 |
| hsa\|miR-214\|as\| | 947 | 1092 | 1.1530 |
| hsa\|miR-383\|as\| | 506 | 530 | 1.0477 |
| hsa\|miR-339\|as\| | 1540 | 1610 | 1.0456 |
| hsa\|miR-130b\|as\| | 787 | 822 | 1.0454 |
| hsa\|miR-181d\|as\| | 1514 | 1574 | 1.0402 |
| hsa\|miR-15b\|as\| | 2684 | 2777 | 1.0345 |
| hsa\|miR-148a\|as\| | 2173 | 2157 | 0.9926 |
| hsa\|miR-199b\|as\| | 1174 | 1159 | 0.9866 |
| hsa\|miR-181b\|as\| | 1482 | 1434 | 0.9677 |
| hsa\|miR-494\|as\| | 5115 | 4782 | 0.9350 |
| hsa\|miR-324-3p\|as\| | 602 | 559 | 0.9296 |
| hsa\|miR-186\|as\| | 1009 | 912 | 0.9043 |
| hsa\|miR-107\|as\| | 12452 | 11189 | 0.8985 |
| hsa\|miR-103\|as\| | 11659 | 10428 | 0.8944 |
| hsa\|miR-142-5p\|as\| | 1843 | 1529 | 0.8295 |
| hsa\|miR-30d\|as\| | 2379 | 1956 | 0.8220 |
| hsa\|let-7a\|as\| | 5339 | 4355 | 0.8158 |
| hsa\|let-7d\|as\| | 5689 | 4530 | 0.7963 |
| hsa\|miR-193b\|as\| | 1702 | 1346 | 0.7909 |
| hsa\|miR-30a-5p\|as\| | 1163 | 918 | 0.7887 |
| hsa\|miR-365\|as\| | 1870 | 1471 | 0.7867 |
| hsa\|let-7g\|as\| | 1876 | 1460 | 0.7786 |
| hsa\|miR-30b\|as\| | 7701 | 5946 | 0.7721 |
| hsa\|let-7f\|as\| | 5633 | 4324 | 0.7677 |
| hsa\|miR-191\|as\| | 8623 | 6485 | 0.7520 |
| hsa\|miR-342\|as\| | 2239 | 1667 | 0.7445 |
| hsa\|miR-206\|as\| | 836 | 614 | 0.7342 |
| hsa\|miR-27b\|as\| | 688 | 504 | 0.7331 |
| hsa\|miR-142-3p\|as\| | 3797 | 2768 | 0.7290 |
| hsa\|miR-361\|as\| | 688 | 499 | 0.7245 |
| hsa\|miR-99a\|as\| | 3075 | 2078 | 0.6759 |
| hsa\|miR-130a\|as\| | 1023 | 686 | 0.6714 |
| hsa\|let-7c\|as\| | 3074 | 2039 | 0.6633 |
| hsa\|miR-100\|as\| | 2194 | 1452 | 0.6618 |
| hsa\|miR-34a\|as\| | 1859 | 1222 | 0.6572 |
| hsa\|let-7b\|as\| | 1501 | 922 | 0.6144 |
| hsa\|miR-16\|as\| | 61194 | 36630 | 0.5986 |
| hsa\|miR-29b\|as\| | 16774 | 9985 | 0.5952 |
| hsa\|miR-26b\|as\| | 6293 | 3667 | 0.5828 |
| hsa\|miR-181c\|as\| | 1826 | 1055 | 0.5779 |
| hsa\|miR-181a\|as\| | 3903 | 2214 | 0.5673 |
| hsa\|miR-21\|as\| | 6177 | 3451 | 0.5587 |
| hsa\|miR-30c\|as\| | 8417 | 4671 | 0.5550 |
| hsa\|miR-155\|as\| | 5814 | 3165 | 0.5443 |
| hsa\|miR-27a\|as\| | 1621 | 870 | 0.5368 |
| hsa\|miR-125b\|as\| | 3177 | 1639 | 0.5160 |
| hsa\|miR-23b\|as\| | 4609 | 2341 | 0.5079 |
| hsa\|miR-24\|as\| | 2041 | 1016 | 0.4978 |
| hsa\|miR-26a\|as\| | 14901 | 7315 | 0.4909 |
| hsa\|miR-29a\|as\| | 15997 | 7787 | 0.4868 |
| hsa\|miR-195\|as\| | 4875 | 2276 | 0.4668 |
| hsa\|miR-23a\|as\| | 4495 | 2043 | 0.4546 |
| hsa\|miR-15a\|as\| | 8820 | 3570 | 0.4047 |
| hsa\|miR-29c\|as\| | 5703 | 1923 | 0.3372 |
| hsa\|let-7e\|as\| | 492 | 0 | 0.0000 |
| hsa\|miR-146a\|as\| | 1462 | 0 | 0.0000 |
| hsa\|miR-150\|as\| | 2017 | 0 | 0.0000 |
| hsa\|miR-210\|as\| | 597 | 0 | 0.0000 |
| hsa\|miR-22\|as\| | 872 | 0 | 0.0000 |
| hsa\|miR-223\|as\| | 1333 | 0 | 0.0000 |
| hsa\|miR-30e-3p\|as\| | 455 | 0 | 0.0000 |
| hsa\|miR-30e-5p\|as\| | 574 | 0 | 0.0000 |
| hsa\|miR-451\|as\| | 440 | 0 | 0.0000 |
| hsa\|miR-99b\|as\| | 517 | 0 | 0.0000 |

TABLE 2

| | 118 (high Myc | 119 (high | 120 (low | 121 (low | | | | | mean fold |
|---|---|---|---|---|---|---|---|---|---|
| Name | tumor) | Myc tumor) | Myc tumor) | Myc tumor) | 118/120 | 118/121 | 119/120 | 119/121 | change |
| mmu\|miR-297\|as\| | 1646 | 506 | 443 | 0 | 3.716 | #DIV/0! | 1.143 | #DIV/0! | #DIV/0! |
| mmu\|miR-298\|as\| | 992 | 913 | 626 | 0 | 1.584 | #DIV/0! | 1.458 | #DIV/0! | #DIV/0! |
| mmu\|miR-324-3p\|as\| | 940 | 622 | 589 | 0 | 1.595 | #DIV/0! | 1.055 | #DIV/0! | #DIV/0! |
| mmu\|miR-351\|as\| | 489 | 477 | 0 | 0 | #DIV/0! | #DIV/0! | #DIV/0! | #DIV/0! | #DIV/0! |
| mmu\|miR-7\|as\| | 1316 | 941 | 516 | 0 | 2.552 | #DIV/0! | 1.826 | #DIV/0! | #DIV/0! |
| mmu\|miR-468\|as\| | 3184 | 1287 | 1020 | 982 | 3.123 | 3.243 | 1.262 | 1.311 | 2.235 |
| mmu\|miR-206\|as\| | 3776 | 1231 | 1350 | 988 | 2.797 | 3.820 | 0.912 | 1.245 | 2.194 |
| mmu\|miR-20\|as\| | 30825 | 29701 | 18475 | 12051 | 1.668 | 2.558 | 1.608 | 2.465 | 2.075 |
| mmu\|miR-370\|as\| | 1205 | 1280 | 664 | 562 | 1.815 | 2.145 | 1.929 | 2.280 | 2.042 |
| mmu\|miR-17-5p\|as\| | 23537 | 23423 | 15248 | 10310 | 1.544 | 2.283 | 1.536 | 2.272 | 1.909 |
| mmu\|miR-290\|as\| | 2097 | 2107 | 1400 | 1024 | 1.497 | 2.048 | 1.505 | 2.058 | 1.777 |
| mmu\|miR-292-5p\|as\| | 1229 | 1160 | 838 | 571 | 1.465 | 2.154 | 1.383 | 2.033 | 1.759 |
| mmu\|miR-346\|as\| | 713 | 1343 | 1082 | 416 | 0.658 | 1.711 | 1.241 | 3.226 | 1.709 |
| mmu\|miR-18\|as\| | 2207 | 2413 | 1374 | 1377 | 1.607 | 1.604 | 1.757 | 1.753 | 1.680 |
| mmu\|let-7b\|as\| | 3417 | 1863 | 1910 | 1558 | 1.789 | 2.193 | 0.975 | 1.195 | 1.538 |
| mmu\|miR-19a\|as\| | 4587 | 4783 | 3955 | 2593 | 1.160 | 1.769 | 1.209 | 1.845 | 1.496 |
| mmu\|miR-17-3p\|as\| | 1243 | 1421 | 968 | 829 | 1.284 | 1.499 | 1.467 | 1.713 | 1.491 |
| mmu\|miR-15b\|as\| | 2054 | 1335 | 1973 | 828 | 1.041 | 2.481 | 0.676 | 1.613 | 1.453 |
| mmu\|miR-320\|as\| | 2028 | 2293 | 1248 | 1931 | 1.625 | 1.050 | 1.837 | 1.187 | 1.425 |
| mmu\|miR-106a\|as\| | 17817 | 18611 | 14213 | 12141 | 1.254 | 1.468 | 1.309 | 1.533 | 1.391 |
| mmu\|miR-219\|as\| | 815 | 743 | 680 | 494 | 1.199 | 1.649 | 1.092 | 1.503 | 1.361 |
| mmu\|miR-19b\|as\| | 16634 | 17123 | 17340 | 10442 | 0.959 | 1.593 | 0.987 | 1.640 | 1.295 |
| mmu\|miR-188\|as\| | 1227 | 1042 | 784 | 1074 | 1.566 | 1.143 | 1.330 | 0.970 | 1.252 |
| mmu\|miR-134\|as\| | 492 | 607 | 443 | 473 | 1.110 | 1.041 | 1.370 | 1.284 | 1.201 |
| mmu\|miR-181b\|as\| | 1367 | 1146 | 1548 | 789 | 0.883 | 1.731 | 0.740 | 1.452 | 1.201 |
| mmu\|miR-301\|as\| | 562 | 542 | 669 | 357 | 0.841 | 1.573 | 0.810 | 1.516 | 1.185 |
| mmu\|miR-452\|as\| | 926 | 1024 | 805 | 903 | 1.150 | 1.026 | 1.272 | 1.134 | 1.146 |
| mmu\|miR-345\|as\| | 492 | 700 | 592 | 479 | 0.831 | 1.027 | 1.182 | 1.462 | 1.126 |
| mmu\|miR-98\|as\| | 1066 | 942 | 1255 | 728 | 0.849 | 1.464 | 0.750 | 1.294 | 1.089 |
| mmu\|miR-93\|as\| | 4584 | 5079 | 5900 | 3905 | 0.777 | 1.174 | 0.861 | 1.301 | 1.028 |
| mmu\|miR-24\|as\| | 2275 | 2757 | 2831 | 2223 | 0.804 | 1.023 | 0.974 | 1.240 | 1.010 |
| mmu\|miR-130b\|as\| | 981 | 1134 | 1166 | 951 | 0.842 | 1.032 | 0.973 | 1.193 | 1.010 |
| mmu\|miR-130a\|as\| | 707 | 751 | 812 | 654 | 0.871 | 1.082 | 0.925 | 1.150 | 1.007 |
| mmu\|miR-431\|as\| | 1877 | 3743 | 2811 | 2801 | 0.668 | 0.670 | 1.332 | 1.337 | 1.001 |
| mmu\|miR-27b\|as\| | 1166 | 1180 | 1449 | 998 | 0.805 | 1.168 | 0.815 | 1.183 | 0.993 |
| mmu\|miR-16\|as\| | 22732 | 21763 | 30645 | 17761 | 0.742 | 1.280 | 0.710 | 1.225 | 0.989 |
| mmu\|let-7d\|as\| | 3927 | 4379 | 5981 | 3300 | 0.657 | 1.190 | 0.732 | 1.327 | 0.976 |
| mmu\|miR-27a\|as\| | 1249 | 1203 | 1462 | 1120 | 0.854 | 1.115 | 0.823 | 1.074 | 0.966 |
| mmu\|let-7c\|as\| | 3544 | 2989 | 4613 | 2716 | 0.768 | 1.305 | 0.648 | 1.101 | 0.955 |
| mmu\|miR-23a\|as\| | 2231 | 1959 | 3648 | 1649 | 0.611 | 1.352 | 0.537 | 1.188 | 0.922 |
| mmu\|miR-92\|as\| | 1433 | 1378 | 2068 | 1212 | 0.693 | 1.183 | 0.666 | 1.137 | 0.920 |
| mmu\|let-7i\|as\| | 6872 | 6690 | 9882 | 6162 | 0.695 | 1.115 | 0.677 | 1.086 | 0.893 |
| mmu\|let-7a\|as\| | 5669 | 5112 | 8235 | 4858 | 0.688 | 1.167 | 0.621 | 1.052 | 0.882 |
| mmu\|miR-214\|as\| | 1642 | 2085 | 2032 | 2250 | 0.808 | 0.730 | 1.026 | 0.927 | 0.873 |
| mmu\|miR-25\|as\| | 2205 | 1696 | 3488 | 1653 | 0.632 | 1.334 | 0.486 | 1.026 | 0.870 |
| mmu\|let-7e\|as\| | 1234 | 944 | 1240 | 1306 | 0.995 | 0.945 | 0.762 | 0.723 | 0.856 |
| mmu\|let-7f\|as\| | 14037 | 13641 | 21840 | 13515 | 0.643 | 1.039 | 0.625 | 1.009 | 0.829 |
| mmu\|miR-23b\|as\| | 2053 | 2130 | 3895 | 1873 | 0.527 | 1.096 | 0.547 | 1.137 | 0.827 |
| mmu\|miR-106b\|as\| | 3959 | 3364 | 5555 | 3849 | 0.713 | 1.029 | 0.606 | 0.874 | 0.805 |
| mmu\|miR-103\|as\| | 1121 | 1037 | 1732 | 1143 | 0.648 | 0.981 | 0.599 | 0.908 | 0.784 |
| mmu\|miR-21\|as\| | 2390 | 1755 | 4003 | 2001 | 0.597 | 1.194 | 0.438 | 0.877 | 0.777 |
| mmu\|miR-101b\|as\| | 1137 | 1105 | 1774 | 1236 | 0.641 | 0.920 | 0.623 | 0.894 | 0.769 |
| mmu\|miR-29b\|as\| | 6449 | 4749 | 10370 | 5959 | 0.622 | 1.082 | 0.458 | 0.797 | 0.740 |
| mmu\|miR-451\|as\| | 32852 | 33705 | 66300 | 36046 | 0.496 | 0.911 | 0.508 | 0.935 | 0.713 |
| mmu\|miR-107\|as\| | 962 | 1070 | 1759 | 1232 | 0.547 | 0.781 | 0.608 | 0.868 | 0.701 |
| mmu\|miR-181a\|as\| | 1530 | 1564 | 3790 | 1568 | 0.404 | 0.976 | 0.413 | 0.997 | 0.697 |
| mmu\|miR-30d\|as\| | 1211 | 1326 | 2675 | 1425 | 0.453 | 0.850 | 0.496 | 0.931 | 0.682 |
| mmu\|miR-26b\|as\| | 1574 | 1206 | 2995 | 1554 | 0.526 | 1.013 | 0.403 | 0.776 | 0.679 |
| mmu\|miR-195\|as\| | 4183 | 4821 | 6432 | 7038 | 0.650 | 0.594 | 0.750 | 0.685 | 0.670 |
| mmu\|miR-140*\|as\| | 1356 | 1120 | 3128 | 1315 | 0.433 | 1.031 | 0.358 | 0.852 | 0.669 |
| mmu\|miR-29a\|as\| | 4278 | 3809 | 8677 | 4730 | 0.493 | 0.904 | 0.439 | 0.805 | 0.660 |
| mmu\|miR-350\|as\| | 497 | 541 | 1179 | 606 | 0.422 | 0.820 | 0.459 | 0.893 | 0.648 |
| mmu\|miR-15a\|as\| | 2592 | 2423 | 4102 | 3792 | 0.632 | 0.684 | 0.591 | 0.639 | 0.636 |
| mmu\|miR-30a-5p\|as\| | 1383 | 1429 | 3003 | 1889 | 0.460 | 0.732 | 0.476 | 0.756 | 0.606 |
| mmu\|let-7g\|as\| | 5684 | 5687 | 11740 | 7831 | 0.484 | 0.726 | 0.484 | 0.726 | 0.605 |
| mmu\|miR-30c\|as\| | 3567 | 3326 | 8567 | 4721 | 0.416 | 0.755 | 0.388 | 0.704 | 0.566 |
| mmu\|miR-191\|as\| | 8290 | 5983 | 22059 | 9055 | 0.376 | 0.915 | 0.271 | 0.661 | 0.556 |
| mmu\|miR-142-5p\|as\| | 4509 | 4511 | 9861 | 6955 | 0.457 | 0.648 | 0.457 | 0.649 | 0.553 |
| mmu\|miR-30b\|as\| | 4027 | 3615 | 9673 | 5519 | 0.416 | 0.730 | 0.374 | 0.655 | 0.544 |
| mmu\|miR-210\|as\| | 499 | 0 | 578 | 399 | 0.863 | 1.250 | 0.000 | 0.000 | 0.528 |
| mmu\|miR-424\|as\| | 1416 | 1234 | 3403 | 2006 | 0.416 | 0.706 | 0.363 | 0.615 | 0.525 |
| mmu\|miR-30e\|as\| | 618 | 627 | 1385 | 1052 | 0.446 | 0.587 | 0.452 | 0.596 | 0.520 |
| mmu\|miR-181c\|as\| | 754 | 698 | 2136 | 1057 | 0.353 | 0.713 | 0.327 | 0.661 | 0.514 |
| mmu\|miR-26a\|as\| | 2000 | 1812 | 6350 | 3435 | 0.315 | 0.582 | 0.285 | 0.527 | 0.427 |
| mmu\|miR-142-3p\|as\| | 6512 | 7090 | 16160 | 17193 | 0.403 | 0.379 | 0.439 | 0.412 | 0.408 |
| mmu\|miR-146\|as\| | 1810 | 1484 | 6089 | 3818 | 0.297 | 0.474 | 0.244 | 0.389 | 0.351 |
| mmu\|miR-467\|as\| | 917 | 1735 | 3781 | 5094 | 0.243 | 0.180 | 0.459 | 0.341 | 0.305 |
| mmu\|miR-34a\|as\| | 426 | 0 | 865 | 591 | 0.492 | 0.721 | 0.000 | 0.000 | 0.303 |
| mmu\|miR-140\|as\| | 417 | 0 | 1257 | 1025 | 0.332 | 0.407 | 0.000 | 0.000 | 0.185 |
| mmu\|miR-150\|as\| | 743 | 451 | 5502 | 3118 | 0.135 | 0.238 | 0.082 | 0.145 | 0.150 |
| mmu\|miR-101a\|as\| | 0 | 0 | 767 | 667 | 0.000 | 0.000 | 0.000 | 0.000 | 0.000 |
| mmu\|miR-139\|as\| | 0 | 0 | 418 | 375 | 0.000 | 0.000 | 0.000 | 0.000 | 0.000 |
| mmu\|miR-144\|as\| | 0 | 0 | 741 | 391 | 0.000 | 0.000 | 0.000 | 0.000 | 0.000 |
| mmu\|miR-215\|as\| | 0 | 0 | 509 | 449 | 0.000 | 0.000 | 0.000 | 0.000 | 0.000 |
| mmu\|miR-22\|as\| | 0 | 0 | 523 | 397 | 0.000 | 0.000 | 0.000 | 0.000 | 0.000 |
| mmu\|miR-29c\|as\| | 0 | 0 | 578 | 500 | 0.000 | 0.000 | 0.000 | 0.000 | 0.000 |
| mmu\|miR-342\|as\| | 0 | 0 | 563 | 398 | 0.000 | 0.000 | 0.000 | 0.000 | 0.000 |
| mmu\|miR-466\|as\| | 0 | 0 | 1031 | 1022 | 0.000 | 0.000 | 0.000 | 0.000 | 0.000 |
| mmu\|let-7d*\|as\| | 0 | 762 | 694 | 0 | | | | | |
| mmu\|miR-1\|as\| | 0 | 0 | 866 | 0 | | | | | |
| mmu\|miR-100\|as\| | 0 | 0 | 0 | 0 | | | | | |
| mmu\|miR-10a\|as\| | 0 | 0 | 0 | 0 | | | | | |
| mmu\|miR-10b\|as\| | 0 | 0 | 0 | 0 | | | | | |
| mmu\|miR-122a\|as\| | 429 | 0 | 0 | 0 | | | | | |
| mmu\|miR-124a\|as\| | 0 | 0 | 0 | 0 | | | | | |
| mmu\|miR-125a\|as\| | 0 | 0 | 0 | 0 | | | | | |
| mmu\|miR-125b\|as\| | 0 | 0 | 0 | 0 | | | | | |
| mmu\|miR-126-3p\|as\| | 740 | 0 | 559 | 0 | | | | | |
| mmu\|miR-126-5p\|as\| | 0 | 0 | 0 | 0 | | | | | |
| mmu\|miR-127\|as\| | 0 | 0 | 0 | 0 | | | | | |
| mmu\|miR-128a\|as\| | 0 | 0 | 0 | 0 | | | | | |
| mmu\|miR-128b\|as\| | 0 | 0 | 0 | 0 | | | | | |
| mmu\|miR-129-3p\|as\| | 0 | 0 | 0 | 0 | | | | | |
| mmu\|miR-129-5p\|as\| | 0 | 548 | 450 | 0 | | | | | |
| mmu\|miR-132\|as\| | 0 | 0 | 0 | 0 | | | | | |
| mmu\|miR-133a\|as\| | 0 | 0 | 0 | 0 | | | | | |
| mmu\|miR-133b\|as\| | 0 | 0 | 0 | 0 | | | | | |
| mmu\|miR-135a\|as\| | 0 | 0 | 0 | 0 | | | | | |
| mmu\|miR-135b\|as\| | 0 | 0 | 0 | 0 | | | | | |
| mmu\|miR-136\|as\| | 0 | 0 | 0 | 0 | | | | | |
| mmu\|miR-137\|as\| | 0 | 0 | 0 | 0 | | | | | |
| mmu\|miR-138\|as\| | 0 | 0 | 0 | 0 | | | | | |
| mmu\|miR-141\|as\| | 0 | 0 | 0 | 0 | | | | | |
| mmu\|miR-143\|as\| | 0 | 0 | 0 | 0 | | | | | |
| mmu\|miR-145\|as\| | 0 | 0 | 0 | 0 | | | | | |
| mmu\|miR-148a\|as\| | 0 | 0 | 0 | 0 | | | | | |
| mmu\|miR-148b\|as\| | 0 | 0 | 0 | 0 | | | | | |
| mmu\|miR-149\|as\| | 0 | 0 | 0 | 0 | | | | | |
| mmu\|miR-151\|as\| | 0 | 0 | 0 | 0 | | | | | |
| mmu\|miR-152\|as\| | 0 | 0 | 0 | 0 | | | | | |
| mmu\|miR-153\|as\| | 0 | 0 | 0 | 0 | | | | | |
| mmu\|miR-154\|as\| | 0 | 0 | 0 | 0 | | | | | |
| mmu\|miR-155\|as\| | 0 | 0 | 0 | 0 | | | | | |
| mmu\|miR-182\|as\| | 0 | 0 | 0 | 0 | | | | | |
| mmu\|miR-183\|as\| | 0 | 0 | 0 | 0 | | | | | |
| mmu\|miR-184\|as\| | 0 | 0 | 0 | 0 | | | | | |
| mmu\|miR-185\|as\| | 0 | 0 | 373 | 0 | | | | | |
| mmu\|miR-186\|as\| | 498 | 0 | 686 | 0 | | | | | |
| mmu\|miR-187\|as\| | 0 | 0 | 0 | 0 | | | | | |
| mmu\|miR-189\|as\| | 0 | 0 | 0 | 0 | | | | | |
| mmu\|miR-190\|as\| | 0 | 0 | 0 | 0 | | | | | |
| mmu\|miR-192\|as\| | 0 | 0 | 0 | 0 | | | | | |
| mmu\|miR-193\|as\| | 0 | 0 | 0 | 0 | | | | | |
| mmu\|miR-194\|as\| | 0 | 0 | 596 | 0 | | | | | |
| mmu\|miR-196a\|as\| | 0 | 0 | 0 | 0 | | | | | |
| mmu\|miR-196b\|as\| | 0 | 0 | 0 | 0 | | | | | |
| mmu\|miR-199a*\|as\| | 0 | 0 | 0 | 0 | | | | | |
| mmu\|miR-199a\|as\| | 0 | 0 | 0 | 0 | | | | | |
| mmu\|miR-199b\|as\| | 0 | 0 | 0 | 0 | | | | | |
| mmu\|miR-200a\|as\| | 0 | 0 | 0 | 0 | | | | | |
| mmu\|miR-200b\|as\| | 0 | 0 | 311 | 0 | | | | | |
| mmu\|miR-200c\|as\| | 0 | 0 | 0 | 0 | | | | | |
| mmu\|miR-201\|as\| | 0 | 0 | 0 | 0 | | | | | |
| mmu\|miR-202\|as\| | 0 | 0 | 0 | 0 | | | | | |
| mmu\|miR-203\|as\| | 0 | 0 | 0 | 0 | | | | | |
| mmu\|miR-204\|as\| | 0 | 0 | 0 | 0 | | | | | |
| mmu\|miR-205\|as\| | 0 | 0 | 0 | 0 | | | | | |
| mmu\|miR-207\|as\| | 0 | 488 | 423 | 0 | | | | | |
| mmu\|miR-208\|as\| | 0 | 0 | 0 | 0 | | | | | |
| mmu\|miR-211\|as\| | 0 | 0 | 0 | 0 | | | | | |
| mmu\|miR-212\|as\| | 0 | 0 | 0 | 0 | | | | | |
| mmu\|miR-213\|as\| | 0 | 0 | 399 | 0 | | | | | |
| mmu\|miR-216\|as\| | 0 | 0 | 0 | 0 | | | | | |
| mmu\|miR-217\|as\| | 0 | 0 | 0 | 0 | | | | | |
| mmu\|miR-218\|as\| | 0 | 0 | 0 | 0 | | | | | |
| mmu\|miR-221\|as\| | 0 | 0 | 0 | 0 | | | | | |
| mmu\|miR-222\|as\| | 0 | 0 | 0 | 0 | | | | | |
| mmu\|miR-223\|as\| | 0 | 0 | 0 | 0 | | | | | |
| mmu\|miR-224\|as\| | 0 | 0 | 0 | 0 | | | | | |
| mmu\|miR-28\|as\| | 0 | 0 | 0 | 0 | | | | | |
| mmu\|miR-291-3p\|as\| | 0 | 0 | 0 | 0 | | | | | |
| mmu\|miR-291-5p\|as\| | 0 | 0 | 0 | 0 | | | | | |
| mmu\|miR-292-3p\|as\| | 0 | 0 | 0 | 0 | | | | | |
| mmu\|miR-293\|as\| | 0 | 0 | 0 | 0 | | | | | |
| mmu\|miR-294\|as\| | 1651 | 0 | 330 | 0 | | | | | |
| mmu\|miR-295\|as\| | 0 | 0 | 0 | 0 | | | | | |
| mmu\|miR-296\|as\| | 0 | 0 | 0 | 0 | | | | | |
| mmu\|miR-299\|as\| | 0 | 0 | 0 | 0 | | | | | |
| mmu\|miR-300\|as\| | 0 | 0 | 0 | 0 | | | | | |
| mmu\|miR-302\|as\| | 0 | 0 | 0 | 0 | | | | | |
| mmu\|miR-30a-3p\|as\| | 0 | 0 | 0 | 0 | | | | | |
| mmu\|miR-30e*\|as\| | 0 | 0 | 0 | 0 | | | | | |
| mmu\|miR-31\|as\| | 0 | 0 | 0 | 0 | | | | | |
| mmu\|miR-32\|as\| | 449 | 0 | 410 | 0 | | | | | |
| mmu\|miR-322\|as\| | 0 | 0 | 0 | 0 | | | | | |
| mmu\|miR-323\|as\| | 0 | 0 | 0 | 0 | | | | | |
| mmu\|miR-324-5p\|as\| | 0 | 0 | 0 | 0 | | | | | |
| mmu\|miR-325\|as\| | 0 | 0 | 0 | 0 | | | | | |
| mmu\|miR-326\|as\| | 0 | 0 | 0 | 0 | | | | | |
| mmu\|miR-328\|as\| | 0 | 0 | 0 | 0 | | | | | |
| mmu\|miR-329\|as\| | 0 | 0 | 0 | 0 | | | | | |
| mmu\|miR-33\|as\| | 0 | 0 | 0 | 0 | | | | | |
| mmu\|miR-330\|as\| | 0 | 0 | 0 | 0 | | | | | |
| mmu\|miR-331\|as\| | 0 | 0 | 0 | 0 | | | | | |
| mmu\|miR-335\|as\| | 0 | 0 | 0 | 0 | | | | | |
| mmu\|miR-337\|as\| | 0 | 0 | 0 | 0 | | | | | |
| mmu\|miR-338\|as\| | 0 | 0 | 0 | 0 | | | | | |
| mmu\|miR-339\|as\| | 0 | 0 | 0 | 0 | | | | | |
| mmu\|miR-340\|as\| | 0 | 0 | 0 | 0 | | | | | |
| mmu\|miR-341\|as\| | 434 | 0 | 0 | 0 | | | | | |
| mmu\|miR-344\|as\| | 0 | 0 | 0 | 0 | | | | | |
| mmu\|miR-34b\|as\| | 0 | 0 | 0 | 0 | | | | | |
| mmu\|miR-34c\|as\| | 0 | 0 | 0 | 0 | | | | | |
| mmu\|miR-361\|as\| | 419 | 0 | 498 | 0 | | | | | |
| mmu\|miR-363\|as\| | 562 | 0 | 0 | 0 | | | | | |
| mmu\|miR-365\|as\| | 0 | 0 | 0 | 0 | | | | | |
| mmu\|miR-375\|as\| | 0 | 0 | 0 | 0 | | | | | |
| mmu\|miR-376a\|as\| | 0 | 0 | 0 | 0 | | | | | |
| mmu\|miR-376b\|as\| | 0 | 0 | 0 | 0 | | | | | |
| mmu\|miR-377\|as\| | 0 | 0 | 0 | 0 | | | | | |
| mmu\|miR-378\|as\| | 0 | 0 | 0 | 0 | | | | | |
| mmu\|miR-379\|as\| | 0 | 0 | 0 | 0 | | | | | |
| mmu\|miR-380-3p\|as\| | 0 | 0 | 0 | 0 | | | | | |
| mmu\|miR-380-5p\|as\| | 0 | 0 | 0 | 0 | | | | | |
| mmu\|miR-381\|as\| | 475 | 0 | 0 | 0 | | | | | |
| mmu\|miR-382\|as\| | 0 | 0 | 0 | 0 | | | | | |
| mmu\|miR-383\|as\| | 567 | 0 | 497 | 0 | | | | | |
| mmu\|miR-384\|as\| | 0 | 0 | 0 | 0 | | | | | |
| mmu\|miR-409\|as\| | 0 | 0 | 0 | 0 | | | | | |
| mmu\|miR-410\|as\| | 0 | 0 | 0 | 0 | | | | | |
| mmu\|miR-411\|as\| | 0 | 0 | 0 | 0 | | | | | |
| mmu\|miR-412\|as\| | 0 | 0 | 0 | 0 | | | | | |
| mmu\|miR-425\|as\| | 0 | 0 | 0 | 0 | | | | | |
| mmu\|miR-429\|as\| | 0 | 0 | 0 | 0 | | | | | |
| mmu\|miR-433-3p\|as\| | 0 | 0 | 0 | 0 | | | | | |
| mmu\|miR-433-5p\|as\| | 0 | 0 | 0 | 0 | | | | | |
| mmu\|miR-434-3p\|as\| | 0 | 0 | 0 | 0 | | | | | |
| mmu\|miR-434-5p\|as\| | 0 | 0 | 0 | 0 | | | | | |
| mmu\|miR-448\|as\| | 0 | 0 | 0 | 0 | | | | | |
| mmu\|miR-449\|as\| | 0 | 0 | 0 | 0 | | | | | |
| mmu\|miR-450\|as\| | 0 | 0 | 378 | 0 | | | | | |
| mmu\|miR-463\|as\| | 0 | 0 | 0 | 0 | | | | | |
| mmu\|miR-464\|as\| | 0 | 0 | 0 | 0 | | | | | |
| mmu\|miR-465\|as\| | 0 | 0 | 0 | 0 | | | | | |
| mmu\|miR-469\|as\| | 0 | 0 | 0 | 0 | | | | | |
| mmu\|miR-470\|as\| | 0 | 0 | 0 | 0 | | | | | |
| mmu\|miR-471\|as\| | 0 | 0 | 0 | 0 | | | | | |
| mmu\|miR-7b\|as\| | 0 | 0 | 0 | 0 | | | | | |
| mmu\|miR-9*\|as\| | 0 | 0 | 0 | 0 | | | | | |
| mmu\|miR-9\|as\| | 0 | 0 | 0 | 0 | | | | | |
| mmu\|miR-96\|as\| | 0 | 0 | 0 | 0 | | | | | |
| mmu\|miR-99a\|as\| | 0 | 0 | 0 | 0 | | | | | |
| mmu\|miR-99b\|as\| | 0 | 0 | 0 | 0 | | | | | |

All miRNAs exhibiting a 2-fold or greater upregulation or downregulation in the high Myc state in both human and mouse models were chosen for further analysis. miRNAs that showed a 1.5-fold or greater change in expression in both models were also selected if a) the miRNA or a related family-member is known to be deleted or mutated in cancer or b) a related family-member changed 2-fold or greater in both models.

Remarkably, the predominant consequence of Myc induction in both model systems was widespread repression of miRNA expression. Very few upregulated miRNAs satisfied the criteria for inclusion in the study. Consistent with earlier findings, miRNAs derived from the mir-17 cluster were upregulated greater than 2-fold by Myc in both models. miR-7 was the only additional consistently upregulated miRNA identified by the microarray experiments. However, this miRNA was not detected by northern blotting, so it was not studied further. At least 13 downregulated miRNAs, potentially representing 21 distinct transcription units, satisfied our criteria for inclusion in the study (Table 3).

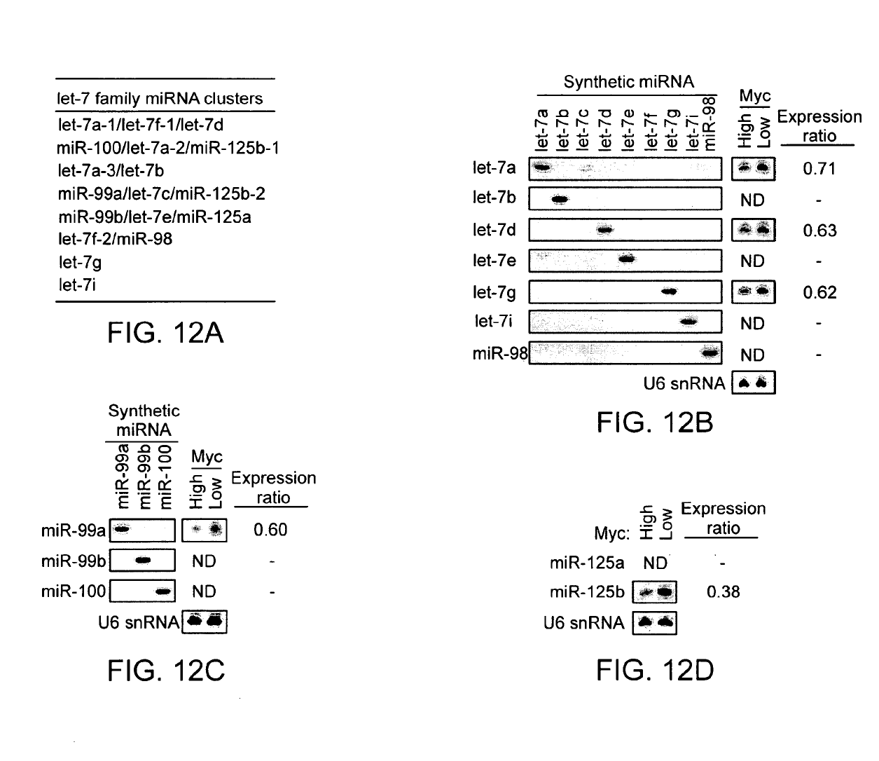

TABLE 3
Candidate Myc-repressed miRNAs identified by microarray

| Criteria | miRNA | Transcription Unit(s)^{a} |
|---|---|---|
| Repressed >2-fold in both models | miR-22 | miR-22 |
| | miR-26a | miR-26a-1; miR-26a-2 |
| | miR-29c | [miR-29b-2/miR-29c] |
| | miR-30e | [miR-30e/miR-30c-1] |
| | miR-146a | miR-146a |
| | miR-150 | miR-150 |
| Repressed >1.5-fold in both models | let-7 | 8 clusters (see FIG. 12A) |
| miRNA or family member deleted or | miR-15a | [miR-15a/miR-16-1] |
| mutated in cancers | miR-29a | [miR-29b-1/miR-29a] |
| | miR-34a | miR-34a |
| | miR-195 | [miR-497/miR-195] |
| Family member repressed >2-fold in | miR-26b | miR-26b |
| both models | miR-30c | [miR-30a/miR-30c-2]; [miR-30e/miR-30c-1] |

^{a} Individual transcription units separated by semi-colon, clustered miRNAs in brackets.

Of these downregulated miRNAs, miR-15a, miR-22, miR-26a, miR-29c, miR-34a, miR-195, and let-7 are mutated or located in genomic regions known to be deleted in cancer (Calin et al., N Engl J Med 353, 1793-801 (2005), Calin et al., Proc Natl Acad Sci USA 101, 2999-3004 (2004)).

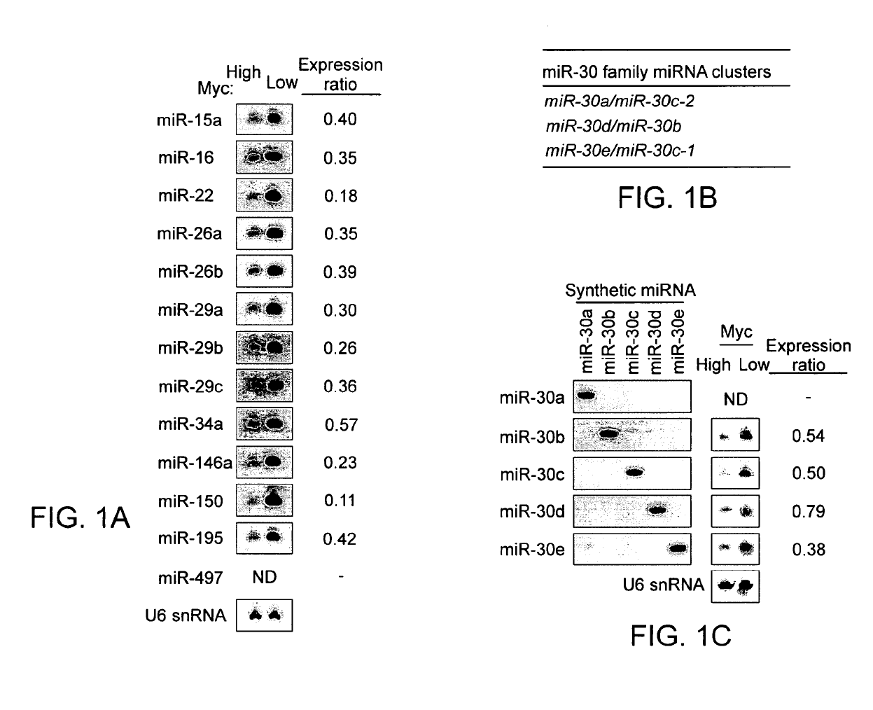

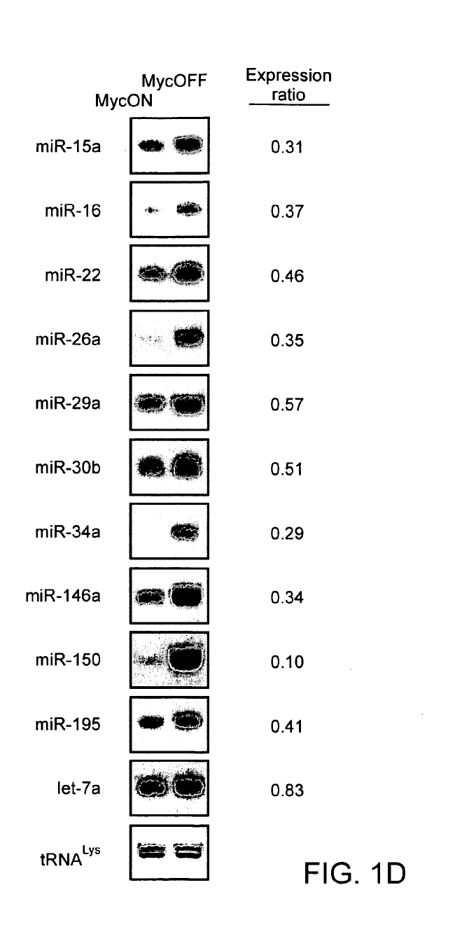

In order to confirm the expression changes detected by microarray analyses, northern blotting was used to examine miRNA expression in P493-6 cells with high (−tet) and low Myc expression (+tet) (FIGS. 1A-1C). In cases where multiple members of a miRNA family showed expression changes (miR-26a/b, miR-29a/c, miR-30e/c, and members of the let-7 family), the possibility that cross-hybridization contributed to the microarray signals was considered. It was previously established that northern blotting conditions that can specifically assay members of the miR-29 family which differ by as few as two nucleotides (Hwang Science 315, 97-100 (2007)). These conditions were used to assay expression of miR-26a and miR-26b, which differ by three nucleotides, and all other miRNAs with the exception of the more complex miR-30 and let-7 families (FIG. 1A). In all cases, the results obtained by northern blotting were highly concordant with those obtained by microarray. All additional miRNAs that are in clusters with downregulated miRNAs were included in these northern blotting studies (miR-16, miR-29b, and miR-497). In most cases, clustered miRNAs behaved similarly (e.g. miR-29a/b, miR-29b/c, miR-15a/miR-16) with the exception of miR-497 which is clustered with miR-195 and was undetectable by microarray and northern.

For the larger miR-30 and let-7 families, additional experiments were performed to establish specific hybridization conditions for each family member. Because of the significant complexity of the let-7 family, analysis of this group of miRNAs will be described separately later in this report. The miR-30 family consists of five distinct mature miRNA sequences (miR-30a-e) organized in three clusters (FIG. 1B). Specific northern blotting conditions were established by hybridizing probes to synthetic RNA oligonucleotides identical in sequence to each miR-30 family member (FIG. 1C). Endogenous miR-30a was not detectable, suggesting that the miR-30a/miR-30c-2 cluster is not expressed in this cell line. The other two miR-30 clusters were expressed and downregulated in the high Myc state.

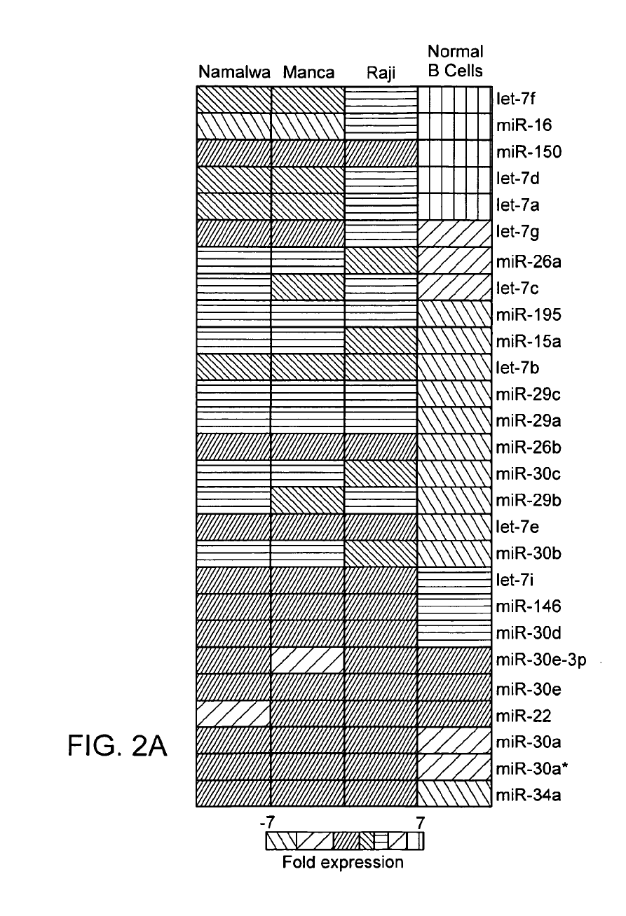

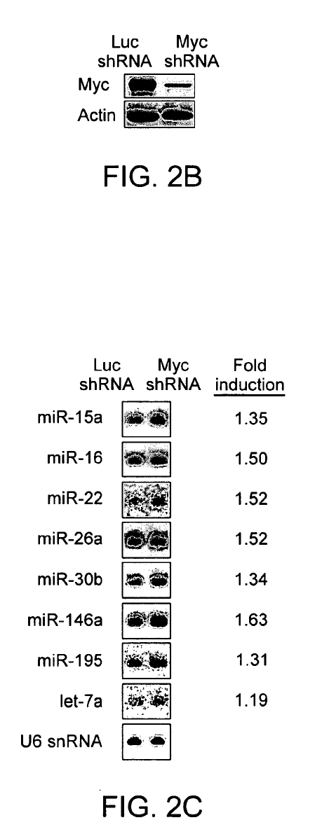

Expression of several miRNAs was further examined in MycER tumors where the expected repression was also observed (FIG. 1D). Next, it was determined whether human tumor cells associated with Myc overexpression exhibit low levels of the putatively repressed miRNAs. Analysis of a previously published miRNA expression profiling dataset (He et al., Nature 435, 828-33 (2005)) revealed that most Myc-repressed miRNAs were expressed at lower levels in Burkitt's lymphoma cells than in non-transformed B cells (FIG. 2A). Moreover, inhibition of Myc expression using short-hairpin RNA (shRNA) in a Burkitt's lymphoma cell line resulted in a modest but consistent upregulation of these miRNAs (FIGS. 2B, C).

### Example 2

### Association of Myc with Promoters of pri-miRNAs

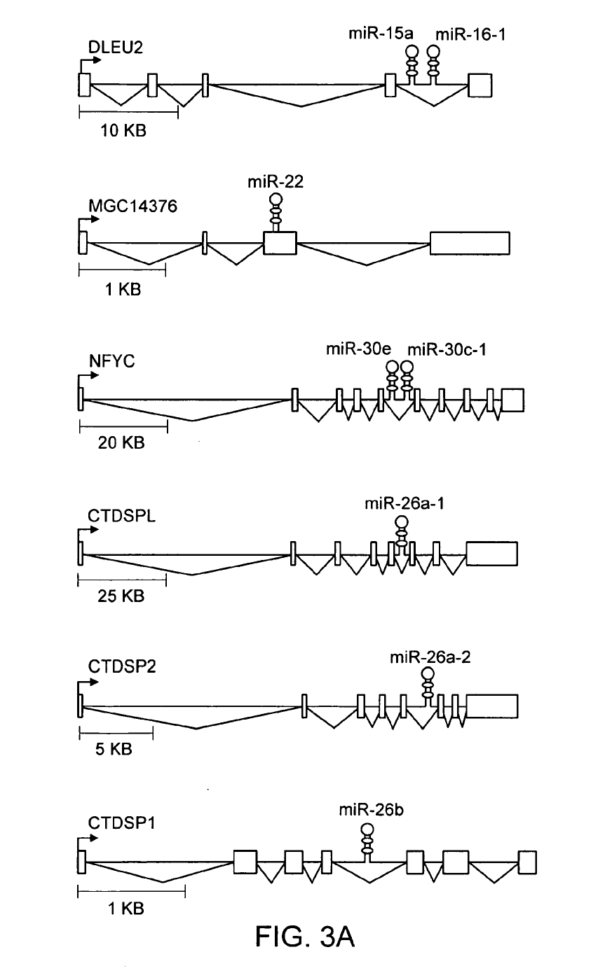

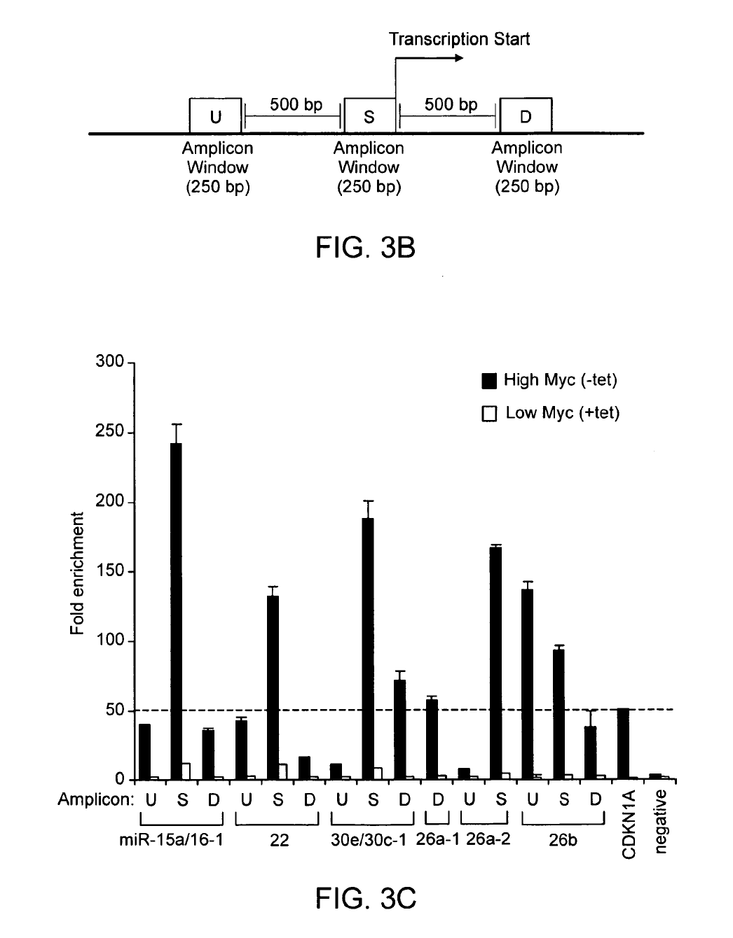

Previous studies have demonstrated that Myc associates with the core promoters of the genes that it represses (Kleine-Kohlbrecher et al., Curr Top Microbiol Immunol 302, 51-62 (2006)). Chromatin immunoprecipitation (ChIP) was used to assay for the presence of Myc at promoters of downregulated miRNAs in P493-6 cells. miRNAs that are contained within pri-miRNAs with previously defined transcription start sites were analysed first. Six such transcripts, encoding 8 miRNAs (miR-15a/16-1, miR-22, miR-30e/30c-1, miR-26a-1, miR-26a-2, and miR-26b), are putative negative targets of Myc based on expression studies reported herein (FIG. 3A). Of note, a genome-wide analysis of Myc binding sites previously revealed association of Myc with the promoter of DLEU2, the miR-15a/16-1 primary transcript (Mao et al., Curr Biol 13, 882-6 (2003)). While expression of the miRNAs was not examined, expression of DLEU2 was found to be reduced in the high Myc state. To assay for Myc binding, real-time polymerase chain reaction (PCR) amplicons were designed within three 250 base pair (bp) windows near the transcription start sites of these miRNA transcripts: Amplicon S, immediately upstream of the transcription start site; amplicon U, located 500 bp upstream of amplicon S; and amplicon D, located 500 bp downstream of amplicon S (FIG. 3B). Due to the high GC content of the promoters for miR-26a-1 and miR-26a-2, only a subset of these amplicons could be designed for these miRNAs. As a positive control, an amplicon was designed within the promoter region of CDKN1A (p21^{WAF1/CIP1}), a validated downregulated target of Myc (Seoane et al., Nature 419, 729-34 (2002)). 50-fold enrichment of the CDKN1A promoter amplicon in Myc ChIP samples was observed as compared to ChIP samples generated with an irrelevant antibody (FIG. 3C). 50-fold enrichment was therefore set as the threshold for positive Myc binding for all subsequent studies. Signals above this threshold were obtained near the transcription start sites for each of the six pri-miRNAs assayed (FIG. 3C), providing strong evidence for association of Myc with these promoters. These signals were dramatically reduced when Myc expression was inhibited by treatment with tet, demonstrating the specificity of these findings.

### Example 4

### Association of Myc with Conserved Regions Upstream of miRNAs

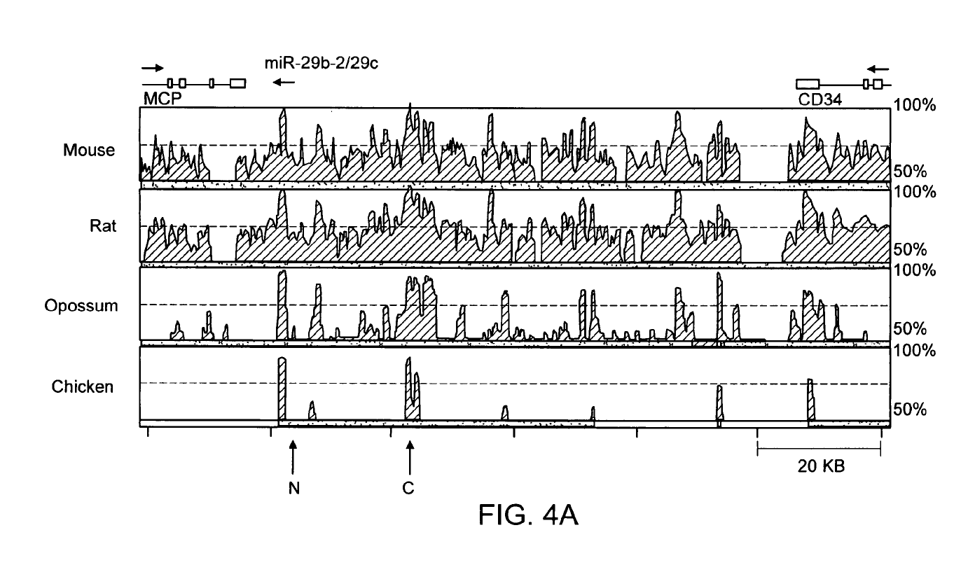

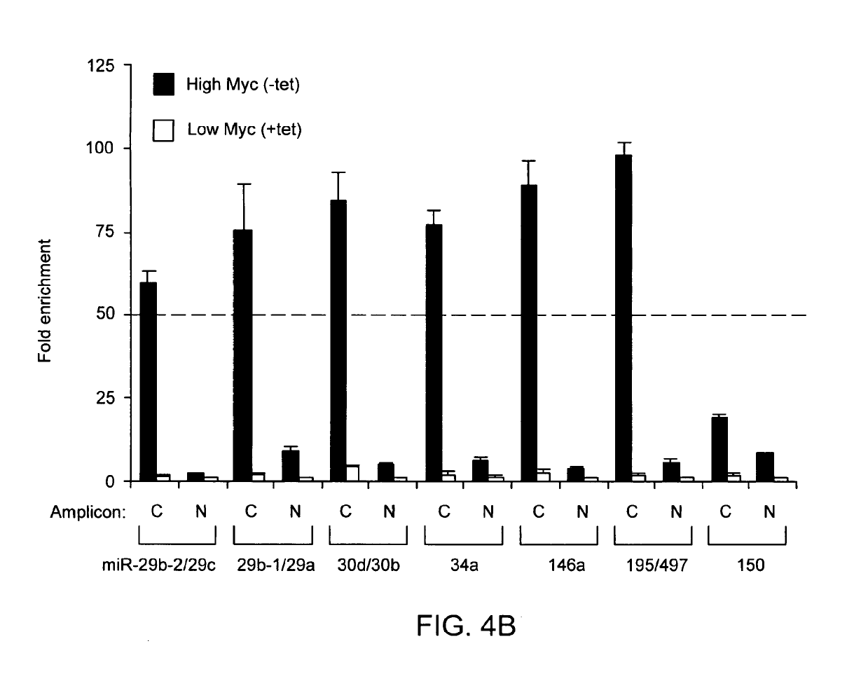

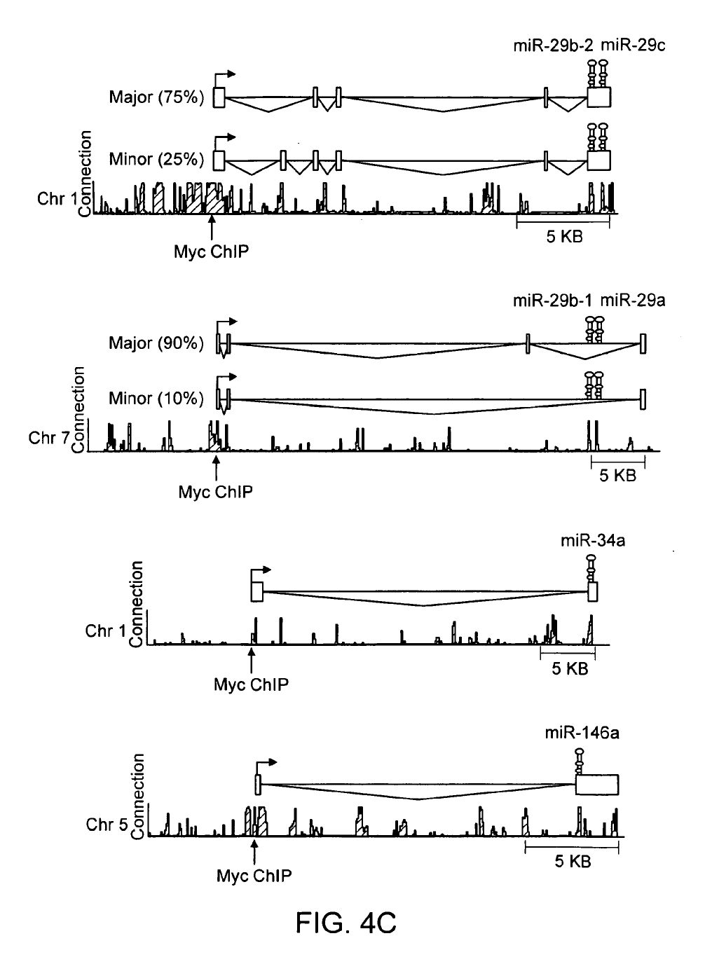

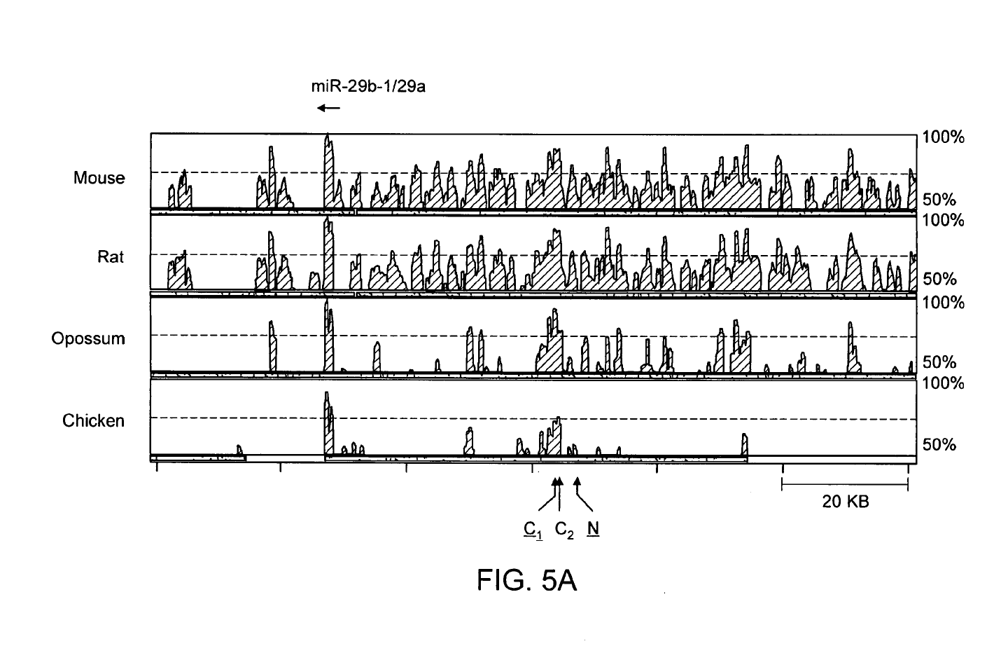

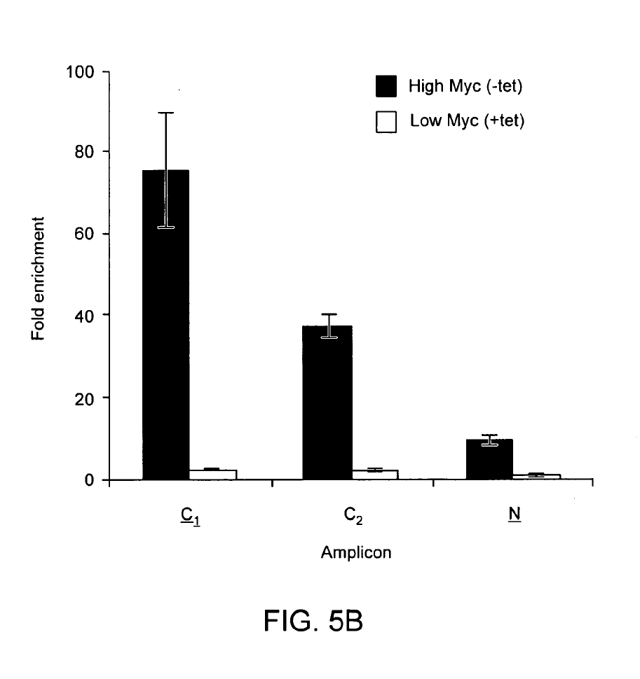

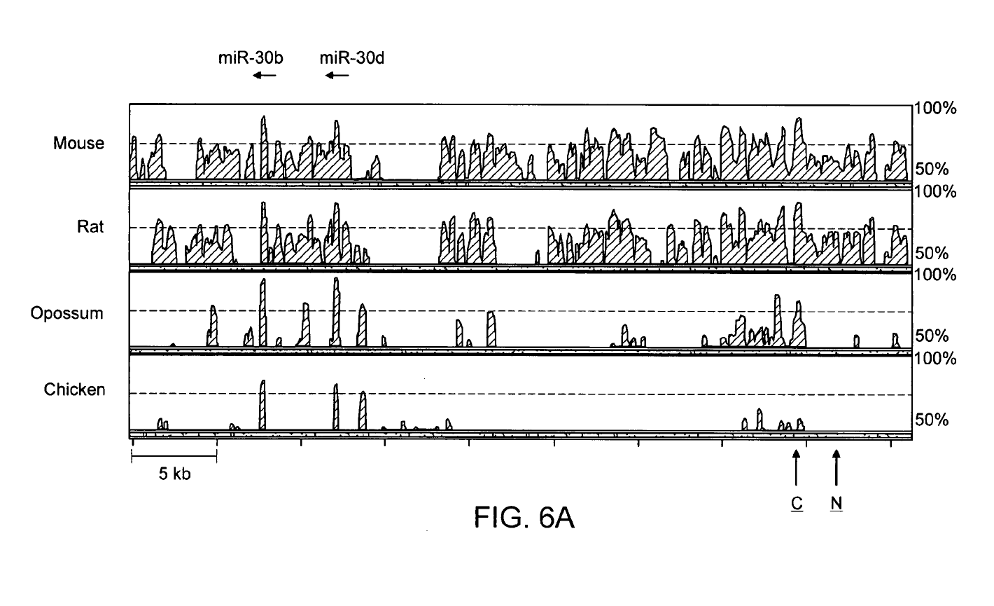

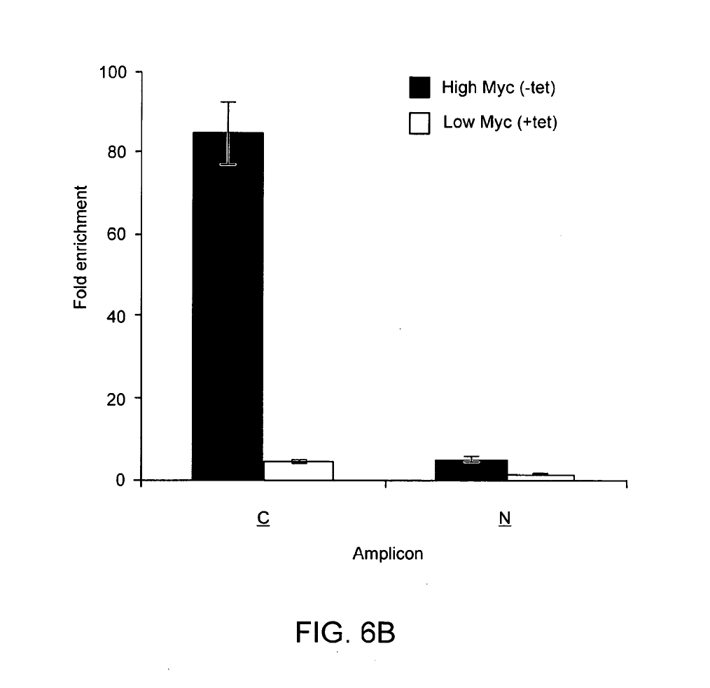

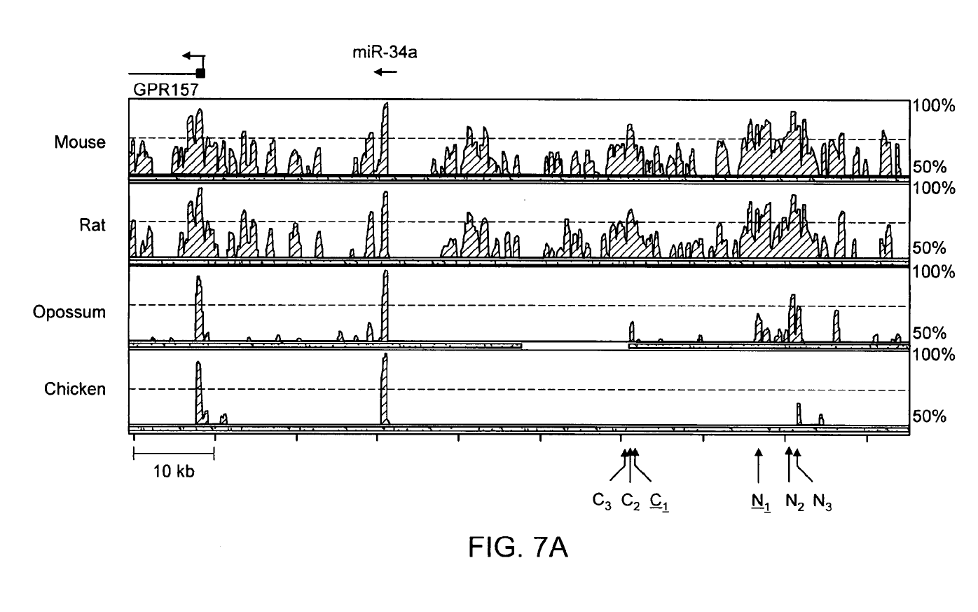

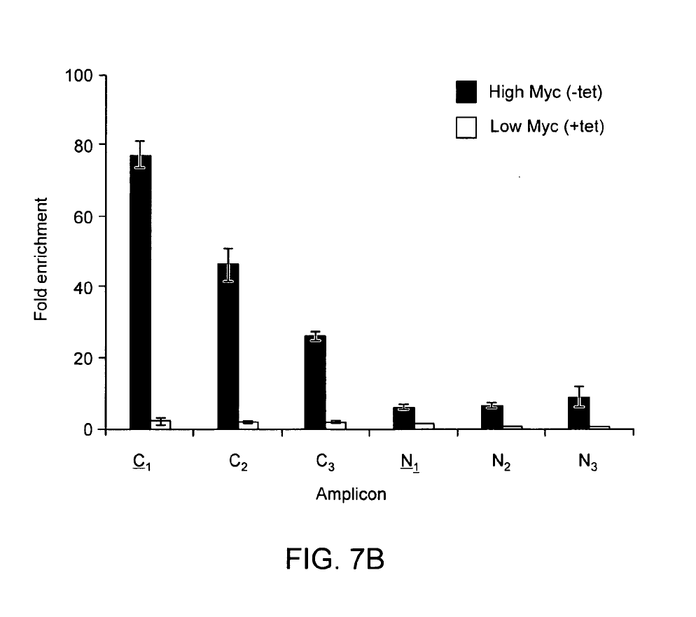

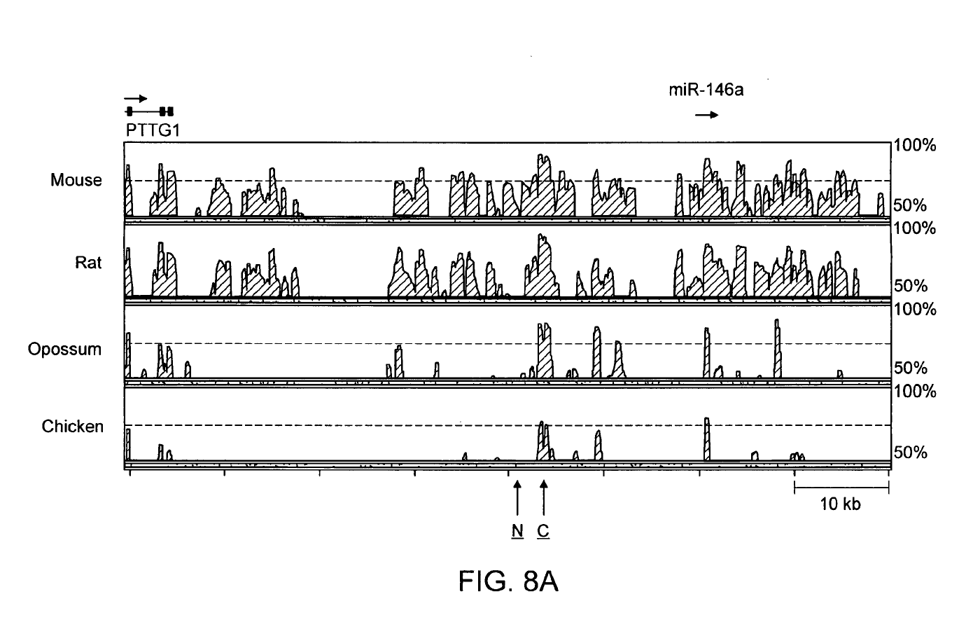

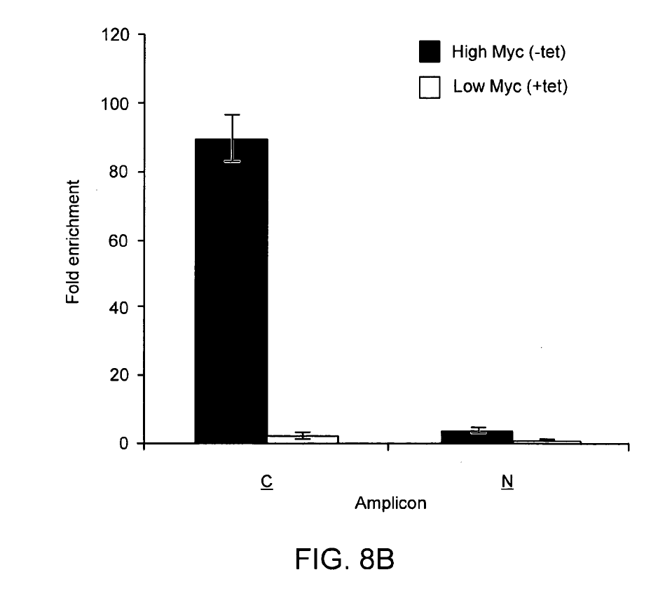

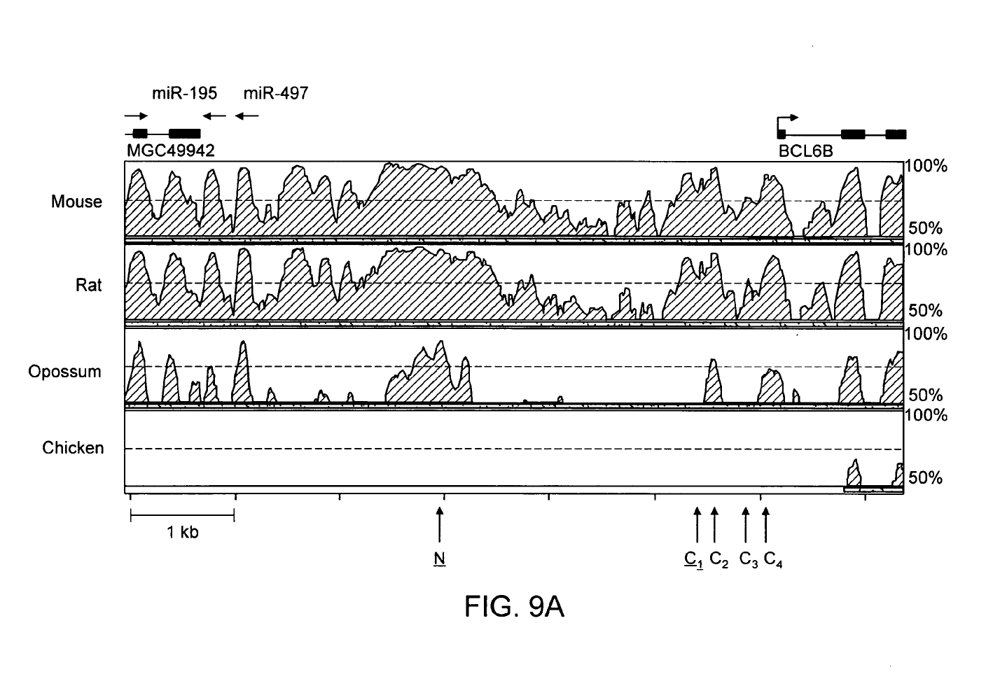

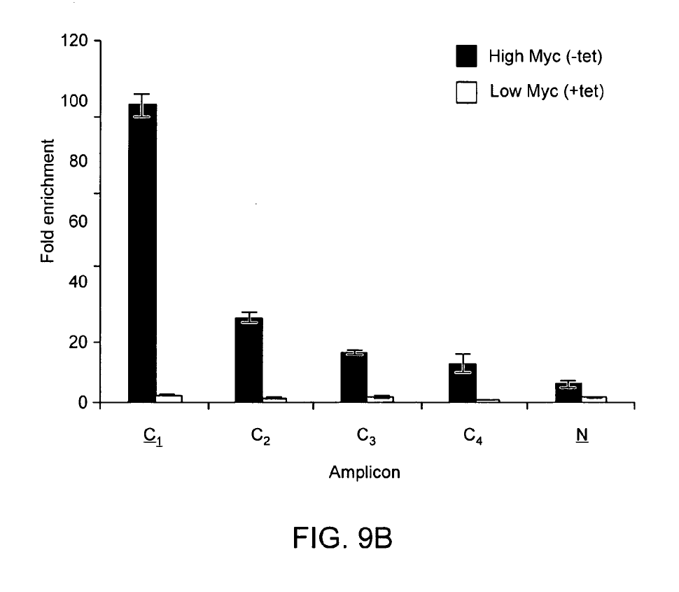

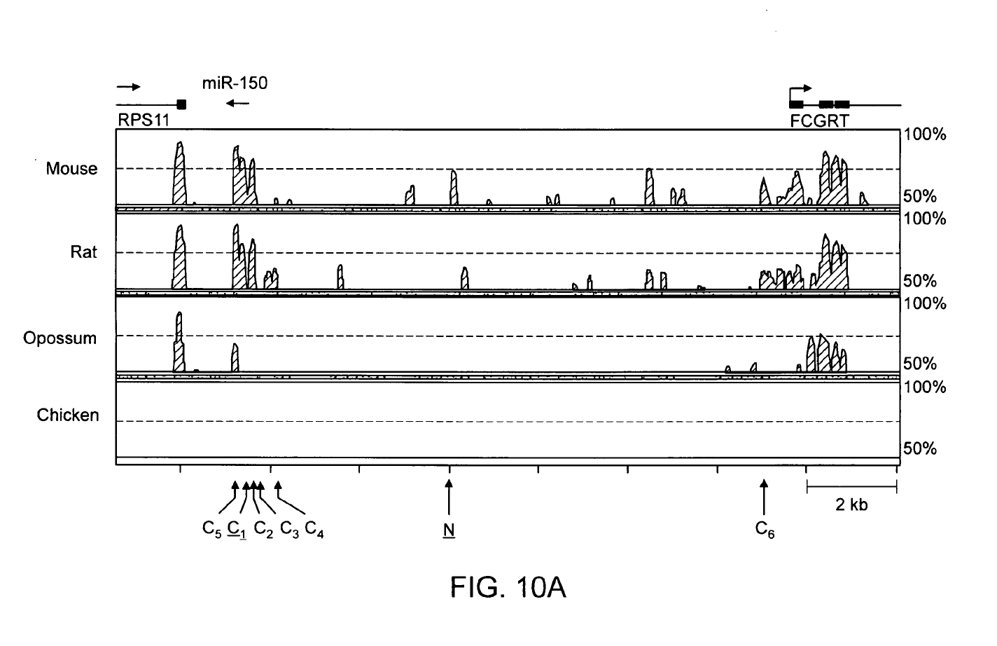

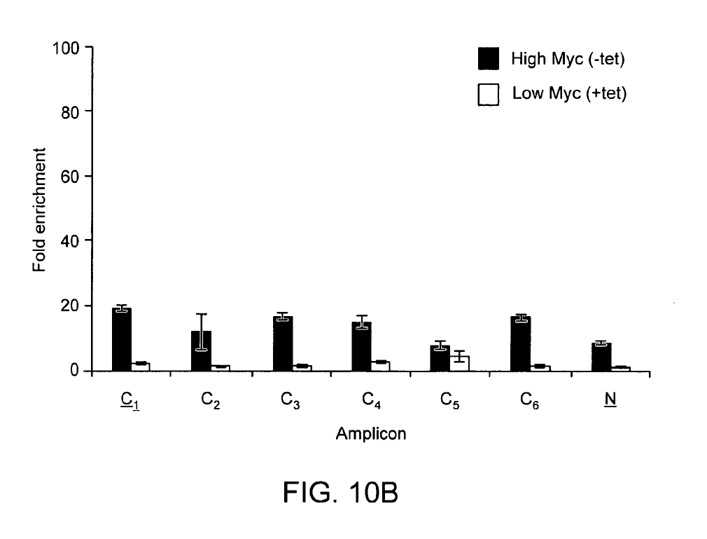

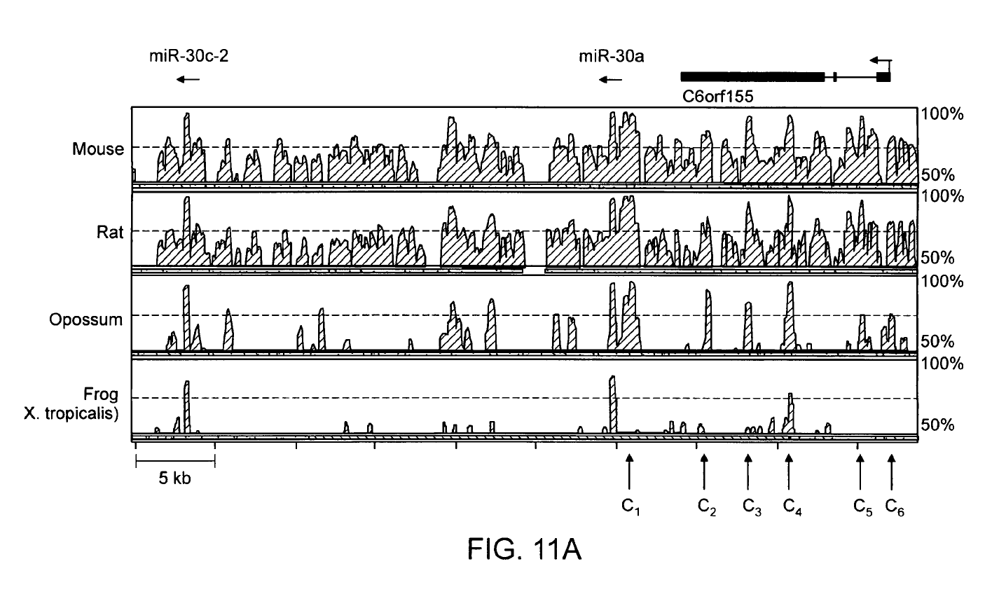

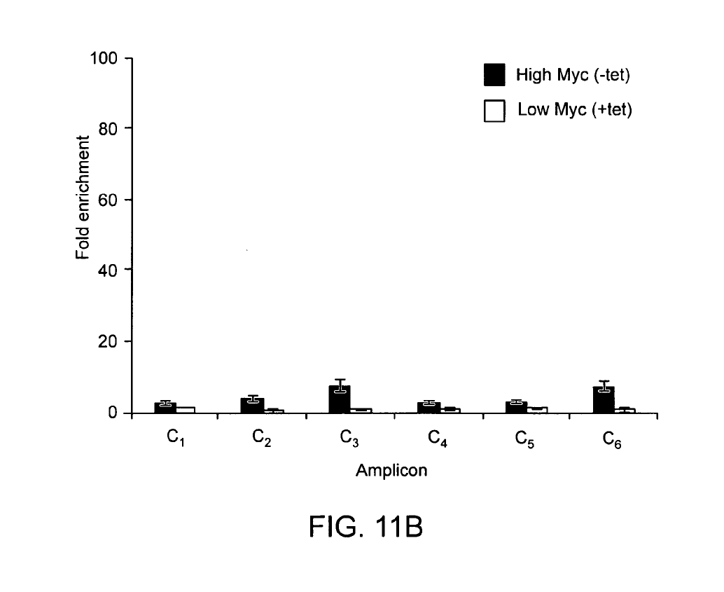

The remaining downregulated miRNAs, with the exception of a subset of the let-7 miRNA clusters, which will be described in detail below, have unmapped transcription start sites and therefore identification of associated Myc binding sites required a different strategy. As illustrated by the pri-miRNAs shown in FIG. 3A, miRNA promoters may be located a few kilobases (kb) to >100 kb upstream of the miRNAs. miRNAs are, in general, highly conserved leading to the hypothesis that promoters would tend to be conserved as well. Conserved candidate regions upstream of miRNAs were therefore selected in which to assess Myc binding. As an initial test of this strategy, the miR-29b-2/29c cluster was examined. Using the Vista software package (http://genome.1bl.gov/vista/index.shtml), a clear region of conservation approximately 20 kb upstream of these miRNAs was identified (FIG. 4A, amplicon C). ChIP analysis in P493-6 cells revealed significant association of Myc specifically with this conserved region (FIG. 4B). Myc was not bound to nearby non-conserved regions (FIG. 4A, amplicon N), demonstrating the specificity of this finding. The same strategy was used to assess Myc binding upstream of the remaining downregulated miRNAs. Evidence was obtained for Myc binding to conserved regions upstream of the miR-29b-1/29a cluster, the miR-30d/30b cluster, miR-34a, and miR-146a (FIG. 4B and FIGS. 5-8). Significant Myc binding was also observed upstream of the miR-195/497 cluster. However, since this binding site is also near the transcription start site for the BCL6B transcript, we cannot rule out the possibility that Myc binding leads to regulation of this transcript, not the miRNAs (FIGS. 9A and 9B). Despite assaying several amplicons, no evidence for Myc binding in the vicinity of miR-150 was obtained (FIGS. 10A and 10B). As a further negative control, Myc binding was assayed at six conserved sites upstream of the miR-30a/30c-2 cluster which is not expressed in P493-6 cells (FIG. 1C). As expected, none of these amplicons yielded positive ChIP signals (FIGS. 11A and 11B).

Given that Myc binds in the vicinity of the transcription start sites of six out of six tested miRNA transcription units of known structure (FIG. 3), it is likely that the conserved Myc binding sites that were identified upstream of miR-29b-1/29a, miR-29b-2/29c, miR-30d/30b, miR-34a, miR-146a, and possibly miR-195/497 are within miRNA promoters. To test this directly, rapid amplification of cDNA ends (RACE) was performed to completely characterize a subset of these pri-miRNAs. For three of these transcripts, spliced expressed sequence tags (ESTs) were available to use as a starting point for RACE. For an additional miRNA, miR-34a, the complete structure of the primary transcript was recently reported (Chang et al., Mol Cell 26, 745-52 (2007)). In each of these cases, the experimentally-determined 5′ end of the pri-miRNA precisely corresponded to the conserved site which exhibited maximal Myc binding (FIG. 4C). Of note, another recently published study defined the identical transcription start site for miR-146a (Taganov et al., Proc Natl Acad Sci USA 103, 12481-6 (2006)). In sum, sites bound by Myc upstream of 12 out of 13 repressed miRNA transcription units of both known and unknown structure were identified. In 10 of these cases, the Myc binding site was determined to precisely correspond to the pri-miRNA 5′ end. These findings indicate that much of the repression of miRNAs observed in the high Myc state is likely to be a direct consequence of Myc binding to miRNA promoters.

### Example 5

### Dissection of the Regulatory Control of let-7 miRNA Clusters

The miRNAs downregulated in the high Myc state included members of the let-7 family which comprises 9 highly related mature miRNA sequences produced from 8 different transcription units (FIG. 12A). Let-7 miRNAs are known to be downregulated in lung tumors and evidence suggests that these miRNAs possess tumor suppressor activity (Johnson et al., Cell 120, 635-47 (2005), Takamizawa et al., Cancer Res 64, 3753-6 (2004), Yanaihara, et al., Cancer Cell 9, 189-98 (2006)). Hybridization conditions specific for nearly all human let-7 miRNAs were established by hybridizing northern probes to synthetic RNA oligonucleotides identical in sequence to each let-7 family member (FIG. 12B). Specific hybridization conditions were also identified for members of the miR-99/100 family which are clustered with a subset of let-7 miRNAs (FIG. 12C). Three let-7 clusters also include members of the miR-125 family, which are sufficiently different to distinguish using standard northern blotting conditions (seven nucleotides differ between miR-125a and miR-125b). Expression of let-7a, let-7d, let-7g, miR-99a, and miR-125b in P493-6 cells were detected and all were downregulated in the high Myc state (FIGS. 12B-12D). The remaining assayed miRNAs were not detectable. These data are most consistent with expression of only the let-7a-1/let-7f-1/let-7d cluster, the miR-99a/let-7c/miR-125b-2 cluster, and let-7g in this cell line.

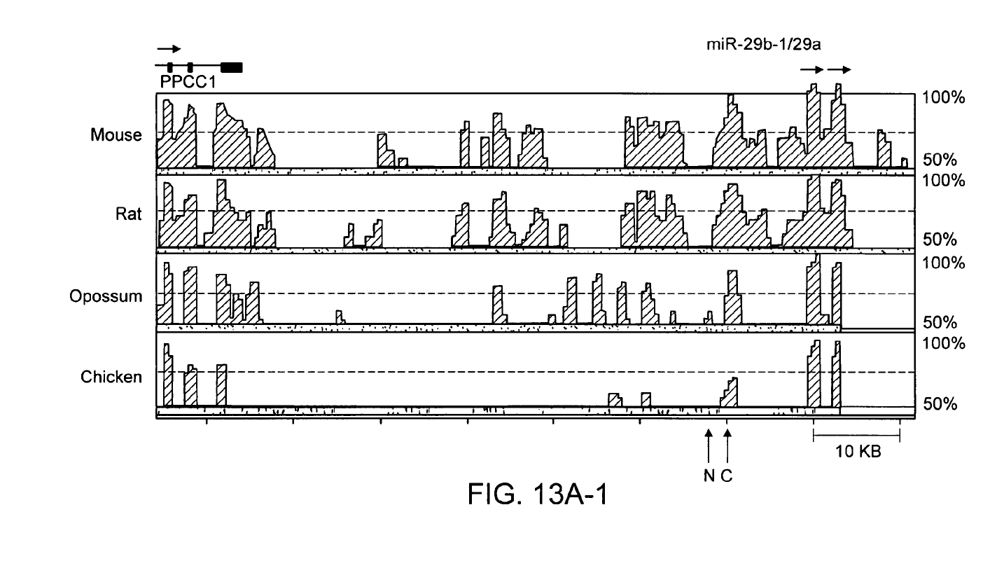

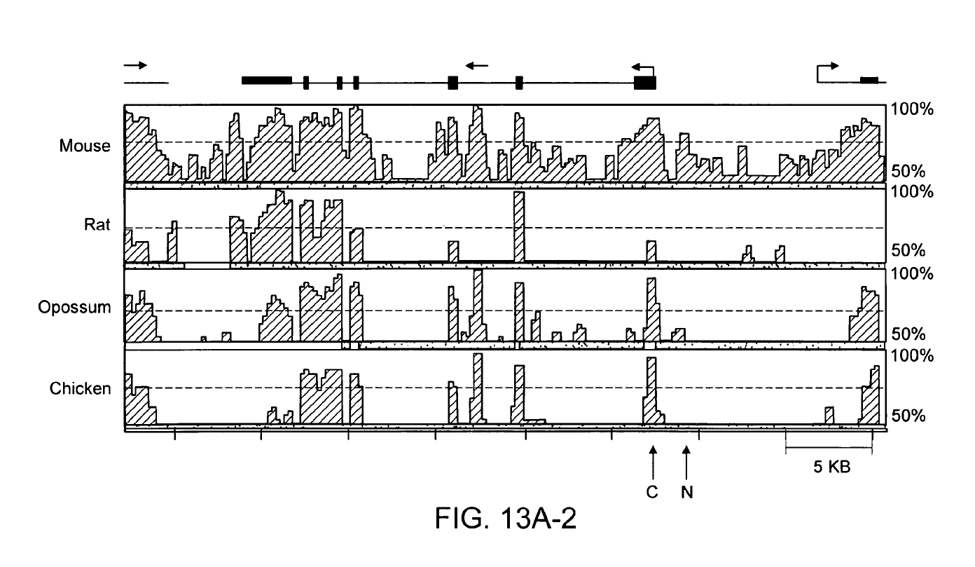

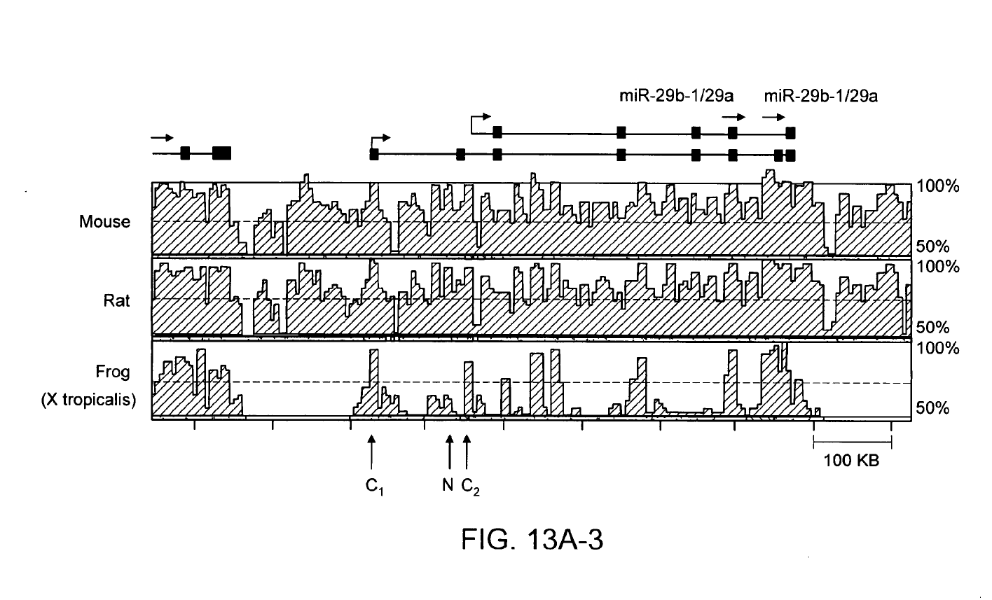

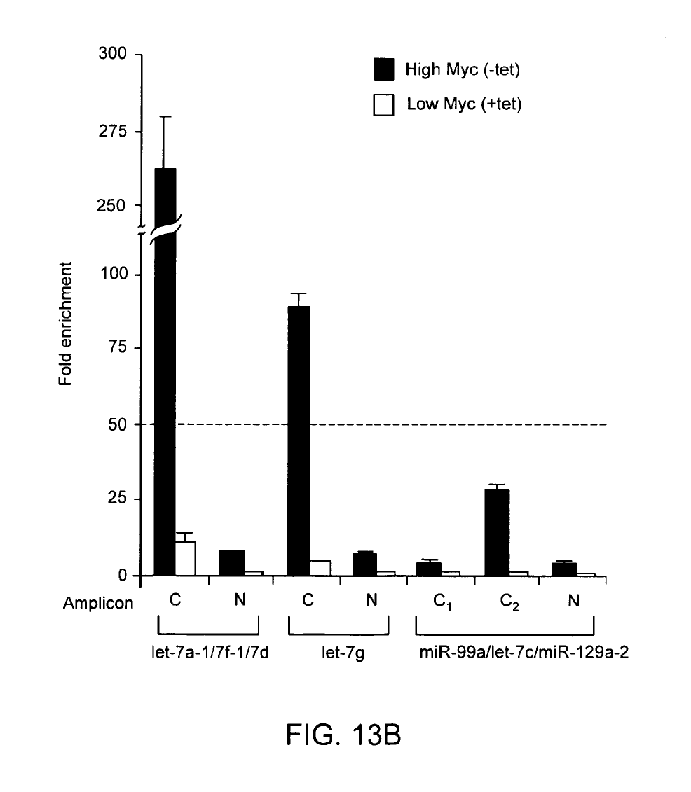

ChIP was again used to assess Myc binding to promoters or conserved sites upstream of these miRNA transcription units. Strong evidence was obtained for Myc binding to a conserved site upstream of the let-7a-1/let-7f-1/let-7d cluster, which is contained within a pri-miRNA that has not been characterized, and to the transcription start site of the let-7g pri-miRNA (FIG. 13). Signals above the 50-fold enrichment threshold were not obtained at either of two alternative transcription start sites for the miR-99a/let-7c/miR-125b-2 pri-miRNA, suggesting that this transcript is not a direct Myc target.

### Example 6

### Expression of Myc-Repressed miRNAs Disadvantages Lymphoma Cell Growth In Vivo

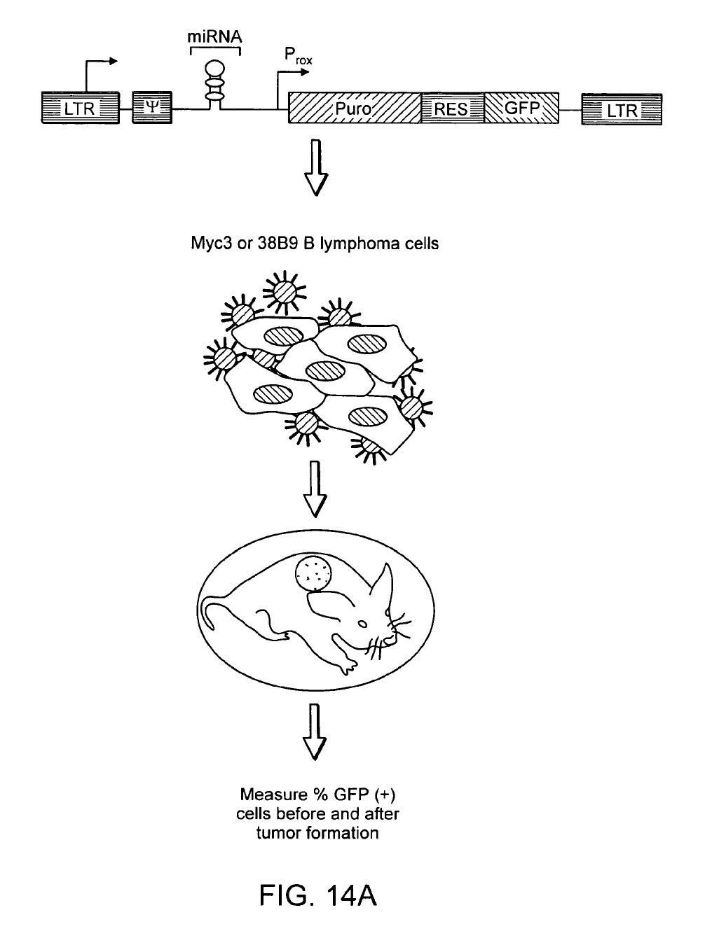

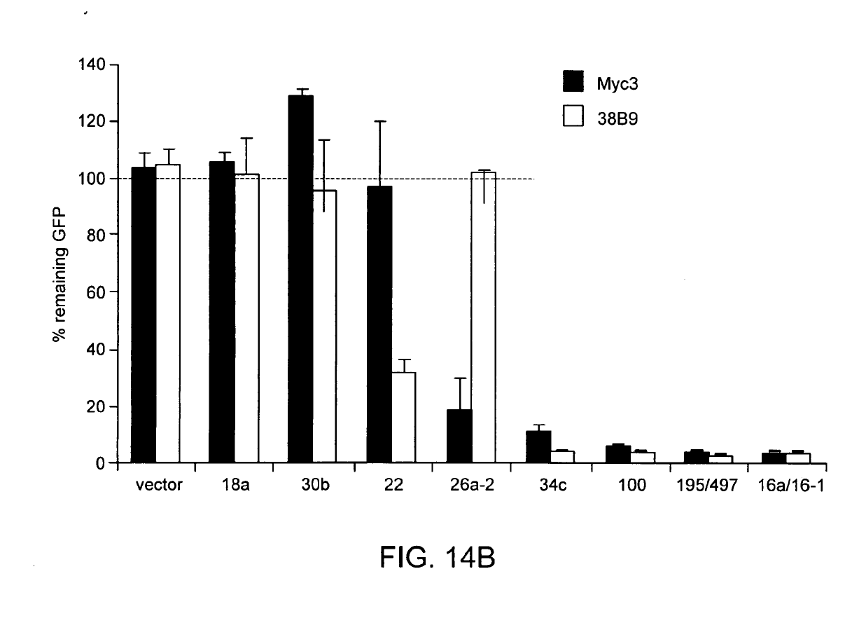

To determine whether downregulation of specific miRNAs contributes to Myc-mediated tumorigenesis, a previously described in vivo selection model of B cell lymphomagenesis was utilized (Yu et al., Ann N Y Acad Sci 1059, 145-59 (2005)). Retroviral expression vectors were first generated by cloning individual human miRNAs or miRNA clusters into a derivative of the murine stem cell virus (MSCV-PIG), which also expresses green fluorescent protein (GFP) (FIG. 14A) (Hemann et al., Nat Genet 33, 396-400 (2003)). 10 distinct miRNA expression constructs were generated (miR-15a/16-1, miR-22, miR-26a-2, miR-29b-1/29a, miR-30b, miR-34a, miR-146a, miR-150, miR-195/497, and let-7a-1/let-7f-1). This set included all unique miRNAs that were downregulated in the high Myc state and at least one member of each downregulated miRNA family. Each of the mature miRNA sequences is identical between human and mouse. Retroviral constructs were used to infect Myc3 cells, a B lymphoma cell line generated by expressing Myc in bone marrow from p53^{-/-} mice (Yu et al., Blood 101, 1950-5 (2003)). To determine the consequences of expressing these miRNAs in the setting of transformation by other oncogenes, 38B9 cells, pro-B cells transformed by the v-Abl oncogene (Alt et al., Cell 27, 381-390 (1981)), were used in a parallel series of experiments. Retroviral infection conditions were adjusted to achieve approximately 50% GFP-positive recipient cells and these mixed cultures were injected subcutaneously into SCID mice. After approximately 3 weeks, the resulting tumors were removed and the percentage of remaining GFP-positive cells was measured. Expression of miRNAs that inhibit tumorigenesis will impart a selective disadvantage to retrovirally-infected cells and therefore will result in a decrease in the fraction of GFP-positive cells in tumors.

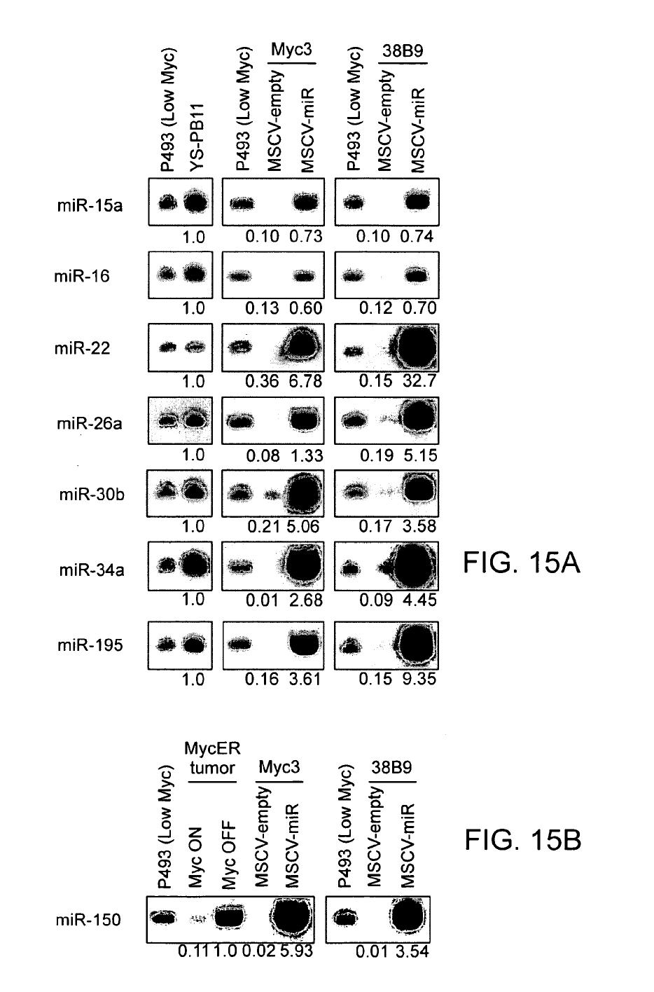

To assess whether retroviral expression produces physiologically-relevant levels of mature miRNAs, the expression levels of miRNAs in retrovirally-infected Myc3 and 38B9 cells was compared to endogenous expression levels in the non-transformed pro-B cell line YS-PB11 (Lu et al., J Immunol 161, 1284-91 (1998)) (FIG. 15). Expression levels of miR-150, which was not expressed in YS-PB11, were compared to MycOFF tumors. In nearly all cases, the level of retroviral miRNA expression ranged from 0.6 to 6 times the level observed in the physiologic setting. Higher levels of expression were obtained with miR-22 in both cell lines and miR-195 in 38B9 cells and therefore results obtained with these viruses in these settings must be interpreted with caution.

Stably-infected cell populations with the let-7a-1/let-7f-1, miR-29b-1/29a, and miR-146a viruses were unable to be established. This may indicate that these miRNAs imposed strong negative selection during in vitro cell growth, although it is also possible that this was a consequence of inefficient packaging of these viruses. For the remaining viruses, 30-70% infection of recipient cells was attained, as assessed by GFP-positivity. The fraction of GFP-positive cells in Myc3 and 38B9 cell populations infected with empty, miR-18a, or miR-30b viruses remained constant before and after tumor formation (FIG. 14B). In contrast, Myc3 or 38B9 cells infected with the miR-34a, miR-150, miR-195/497, and miR-15a/16-1 viruses were nearly eliminated from tumors, indicating that these miRNAs possess anti-tumorigenic properties in the setting of both Myc- and v-Abl-mediated transformation. miR-26a inhibited tumorigenesis specifically in Myc-transformed cells whereas miR-22 expression only affected tumorigenesis in v-Abl-transformed cells. Importantly, there was no correlation between the magnitude of miRNA expression and the phenotype observed, indicating that these results are unlikely to represent an artifact of retroviral overexpression. For example, miR-15a/16-1, which had one of the strongest negative effects on tumorigenesis in both cell lines, exhibited the lowest level of retroviral expression (FIGS. 15A and 15B). These data demonstrate that several of the miRNAs that Myc represses have tumor suppressing activity both in the setting of Myc-mediated transformation as well as in the context of transformation by other oncogenes.

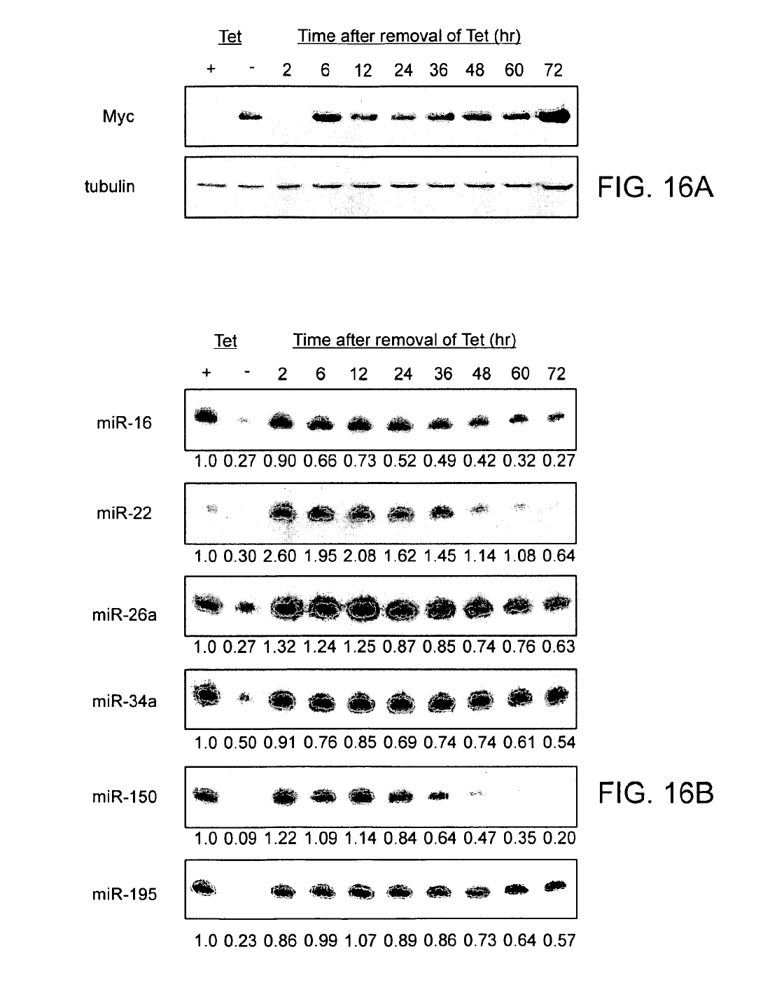

In order to determine whether downregulation of anti-tumorigenic miRNAs correlates with enhanced cellular proliferation following Myc activation, the kinetics of miRNA repression in P493-6 cells was examined (FIG. 16). These cells do not begin proliferating until 48 hours after tet removal and do not reach maximal growth rates until at least 72 hours after Myc induction (O'Donnell et al., Mol Cell Biol 26, 2373-86 (2006)). Significant downregulation of miRNAs was observed by these time-points, consistent with a requirement for their repression to precede Myc-induced proliferation.

Pathologically activated expression of Myc is one of the most common oncogenic events in human cancers. In this study, a major consequence of Myc activation was extensive reprogramming of the miRNA expression pattern of tumor cells. Although the pro-tumorigenic mir-17 cluster was previously shown to be directly upregulated by Myc (O'Donnell et al., Nature 435, 839-43 (2005)), the new findings reported herein unexpectedly reveal that the predominant influence of Myc on miRNA expression is widespread downregulation. Repression of miRNA expression by Myc is consistent with the observation that miRNA levels are globally reduced in tumors. It has been demonstrated that a block in miRNA biogenesis contributes to repression of specific miRNAs in cancer. These new findings indicate that direct transcriptional repression is also likely to contribute to this phenomenon.

Several lines of evidence support the conclusion that miRNA repression favors Myc-mediated tumorigenesis. First, several of the miRNAs downregulated by Myc are mutated or located in regions known to be deleted in cancer, suggesting that they act as tumor suppressors (Calin et al., N Engl J Med 353, 1793-801 (2005); Calin et al., Proc Natl Acad Sci USA 101, 2999-3004 (2004)). miR-15a and miR-16-1 are deleted or downregulated in over two-thirds of patients with chronic lymphocytic leukemia and target the anti-apoptotic gene BCL2. Members of the let-7 miRNA family target the RAS oncogene and are frequently downregulated in lung cancer (Johnson et al., Cell 120, 635-47 (2005), Takamizawa et al., Cancer Res 64, 3753-6 (2004), Yanaihara, et al., Cancer Cell 9, 189-98 (2006)). Recent evidence has implicated miR-34a as critical component of the p53 tumor suppressor network with potent anti-proliferative and pro-apoptotic activity. Repression of these miRNAs by Myc is likely to be an important mechanism contributing to their reduced function in cancer cells. Moreover, as shown herein, several Myc-repressed miRNAs have dramatic anti-tumorigenic activity in a mouse model of B cell lymphoma. For miR-26a, miR-150, and miR-195/497, this represents the first reported experimental data showing that these miRNAs have tumor suppressing properties. Taken together, the available data support an important role for the control of miRNA expression in Myc-mediated tumorigenesis. Furthermore, given recent successes in systemic delivery of small RNAs to animals, these results raise the possibility that delivery of Myc-repressed miRNAs represents a novel therapeutic strategy for cancer. Indeed, these findings indicate that re-expression of even a single critical miRNA may be sufficient to block tumor formation.

This study also highlights the importance of careful dissection of the regulatory control of related miRNAs in cancer as well as in other biological processes. miRNAs frequently exist in multiple highly related or identical copies distributed throughout the genome of a given species. This organization is exemplified by the 9 distinct miRNAs of let-7 family that are produced from 8 individual transcription units in humans. While previous studies have observed downregulation of let-7 miRNAs in cancer (Johnson et al., Cell 120, 635-47 (2005), Takamizawa et al., Cancer Res 64, 3753-6 (2004), Yanaihara, et al., Cancer Cell 9, 189-98 (2006)), the expression of individual let-7 transcription units, and therefore the origin of let-7 miRNAs in a given tumor, has rarely been examined. In this study, the feasibility of dissecting the complex regulatory control of these miRNAs was demonstrated. Since related miRNAs do not always have identical functions (Hwang Science 315, 97-100 (2007)), characterization of the specific miRNA family members that are dysregulated in a given tumor type is a necessary prerequisite for elucidating their roles in cancer pathogenesis.

Finally, these data provide insight into the significance of the nearly ubiquitous dysregulation of miRNA expression that has been observed in diverse cancer subtypes. Our results indicated that these abnormal miRNA expression patterns can not be explained solely as an indirect consequence of the loss of cellular identity that accompanies malignant transformation. Rather, oncogenic events appear to directly reprogram the miRNA transcriptome to favor tumorigenesis.

Results reported herein were obtained using the following materials and methods. Cell culture. P493-6 cells (see, Pajic et al. ((2000). “Cell cycle activation by c-myc in a burkitt lymphoma model cell line,” International Journal of Cancer 87(6):787-93) were cultured in RPMI 1640 media supplemented with 10% fetal bovine serum (FBS), penicillin, and streptomycin. To repress Myc expression, cells were grown in the presence of 0.1 μg/ml tetracycline (Sigma) for 72 hours. Murine lymphoma cells with high and low Myc were obtained as described (Yu et al., Cancer Research 65, 5454-5461 (2005) Yu et al., Oncogene 21, 1922-7 (2002)).

miRNA Microarray Analysis

Custom microarrays containing oligonucleotide probes complementary to 313 human miRNAs or 233 mouse miRNAs were synthesized by Combimatrix. Probes containing 2 mismatches were included for all miRNAs. Array hybridization and data analysis were performed as described (Chang et al., Mol Cell 26, 745-52 (2007)). Signals that were less than 2 times background were removed from subsequent analyses (appear as zero in Tables 1 and 2). For miRNA profiling of murine B cell lymphomas, 2 tumors with high Myc levels and 2 tumors with low Myc levels were analyzed. miRNAs that were absent in ¾ tumors or absent in one of each of the high Myc and low Myc tumors were removed from subsequent analyses. Fold-change values were calculated for all 4 pairwise comparisons between the high Myc and low Myc tumors and then averaged to generate a mean fold-change value.

### Northern Blot Analysis.

For all miRNAs except those of the miR-30, miR-99/100, and let-7 family, northern blotting was performed as described (Hwang Science 315, 97-100 (2007)) using Ultrahyb-Oligo (Ambion) and oligonucleotide probes perfectly complementary to the mature miRNA sequences. To establish specific hybridization conditions for related miRNAs, 1 μl of 10 nM RNA oligonucleotides were separated on polyacrylamide gels and probed as above. Blots were washed once in 2×SSC, 0.5% SDS at 42° and a second time at a higher temperature such that less than 10% cross-hybridization was observed. Specific wash temperatures for each probe are listed in Table 4 (below).

| | Specific wash temperature (° C.) |
|---|---|
| miR-30 family | |
| miR-30a | 44 |
| miR-30b | 44 |
| miR-30c | 48.5 |
| miR-30d | 56 |
| miR-30e | 45.5 |
| let-7 family | |
| let-7a | 58 |
| let-7b | 54 |
| let-7d | 54 |
| let-7e | 44.5 |
| let-7g | 47 |
| let-7i | 47.5 |
| miR-98 | 49.5 |
| miR-99/100 family | |
| miR-99a | 48 |
| miR-99b | 44 |
| miR-100 | 48 |

### Myc Knockdown in Burkitt's Lymphoma Cells.

293T packaging cells were transfected with pLKO.1-Puro lentivirus that expresses anti-Myc shRNA or control shRNA (Sigma). EW36 cells were infected three times with lentiviral supernatant. 48 hours after initial infection, cells were selected in puromycin for 48 hours prior to collection of total RNA and protein.

### Chromatin Immunoprecipitation (ChIP) and Quantitative Real-Time PCR.

ChIP was performed as previously described (O'Donnell et al., Nature 435, 839-43 (2005)) Real-time PCR was performed using an ABI 7900 Sequence Detection System with the SYBR Green PCR core reagent kit (Applied Biosystems). Sequences of primers used to amplify ChIP samples are provided in Table 5 (below).

TABLE 5
Primer sequences for real-time PCR

| | | Forward primer sequence | Reverse primer sequence |
|---|---|---|---|
| miRNA transcription unit | Amplicon | (5′-3′) | (5′-3′) |
| miR-15amiR-16- [symbol] | U | TGGGCACTGTGCTAAATAAATGA | TGAGCAATAAACACGATTAATTCGTAA |
| miR-15amiR-16- [symbol] | S | ATACCGCCTCTTAACCCCCC | CATGCGTAAAAATGTCGGGAA |
| miR-15amiR-16- [symbol] | D | AATCGTTAGCTCGAAGCCCC | GGGAGGAGTGTTCACGGGT |
| miR-22 | U | CTTCTCTCGGCCCAAGACG | AACTCTAACCCCCGCTCCC |
| miR-22 | B | CTGGCTCTGATTGGCAAGGA | TCGTGCAATTCCGCCC |
| miR-22 | D | ACCTTAGGGTAGGGAGGGCT | CATGGCCCATCCCCTAATTT |
| miR-26a-1 | D | GGAGAGCTGGGAGCGAGTGT | CAAACTCACAACCTCCCGGT |
| miR-26a-2 | U | CAACCTTCGAATCCCGAAAG | GAGTCCTAGGTCCGCCCAC |
| miR-26a-3 | [symbol] | CTCCATCTGTGAGCGGCC | AAAATAGCAAAGCTCCCGACTG |
| miR-25b | U | CAAAATAGTAACGACGAGTGAAAAGAA | TGGTCTTTTTCCTCGTTTATGAAGTT |
| miR-25b | [symbol] | GCTCTTGACGTCCTTGCGAG | TTCTCTCCTGTCTGGTGGTCG |
| miR-25b | D | AGGTGAGGAAATGAGGCAGG | AGGAAACCCCCGAAGAGTTC |
| miR-29b-1/miR-29a | C₁ | CACCAACTGAAAACCTGCCA | GAATGAACGTTGTGAAATCCCTC |
| miR-29b-1/miR-29a | C₂ | TGCGCGTGACCAGAAAAGTA | GCCTCAGATTGGTTCGCTTG |
| miR-29b-1/miR-29a | N | CCTTTCACTCCCAGCCCAAT | CCACCATGTGGCTATGACACAG |
| miR-29b-2/miR-29c | C | AGGGAGCCAACATGGAGACA | CGTTGGAAAGTTGTTTACCTTGC |
| miR-29b-2/miR-29c | N | ACTCCAAAGACTGTGTTTCTGCC | TTATGGAGCAGGCTGCAGTG |
| miR-30a/miR-30c-2 | C₁ | AGCAGGTGAAAACAAGCTGAATT | TAGTTAATAAAGAAAAAGGCCACAACAT |
| miR-30a/miR-30c-2 | C₂ | TGAGGTAGAGTGGAAACTGGAGAGA | AACTTAAAAAAAAATTCTTCCATCCTTCT |
| miR-30a/miR-30c-2 | C₃ | AGTGGCATCTTAAAGCAGCACAC | TTTTTCCCTTTTGCATTTTGAGA |
| miR-30a/miR-30c-2 | C₄ | GCACGAATGAATATAAAAACACCAGA | AAGTGCTAAAGCTATGGTTGACTGC |
| miR-30a/miR-30c-2 | C₅ | AGCTGCCTTGGCGTCAGTAA | GAAGGATTGAAAATAGCTACTGTGTTCA |
| miR-30a/miR-30c-2 | C₆ | CCCAATCAGGTGTCGGAAAG | CTATTGGCTACACTCCCGGG |
| miR-30d/miR-30b | O | GCTCCCTCGCCTTTAGTTTGA | GCTCTCCCTCAGACACACTGG |
| miR-30d/miR-30b | N | CCCTCGTCATACTATGGCACG | ACTTCAAGATCATGGTACTGGGC |
| miR-30e/miR-30c-1 | U | TACCATCAGCAGAGGCAGTCA | AGTGCATTAGGTAACAAGCGCA |
| miR-30e/miR-30c-1 | [symbol] | GTCGCCCCTTCCCAATTC | TGCGCAGAAGCTGTGCTC |
| miR-30e/miR-30c-1 | D | TGGCCTGGCAGGTACTTTG | GTGTCCCCCATTCCC |
| miR-34a | C₁ | GACGGGACAGCGGCATC | CCCACCTGGTCCTCTTTCCT |
| miR-34a | C₂ | GGACTCCCGCAAAATCTCC | CTTCTCGGTGACCACGCAG |
| miR-34a | C₂ | AACATTTTGTTGCTTCTTGGAAATT | AATTGTGTAGCCTCCGTAAGGG |
| miR-34a | N₁ | CCTCCACGGTGGAGATGCT | GTTGCTTTTTCCTGTCCCCA |
| miR-34a | N₂ | AAAGCTGCAGTGTCCAAATTCTC | CTGATGTCGGTGACAGTGGG |
| miR-34a | N₂ | GGCAGGACCCGAAATAAGAAG | CACCATTTGGGTGCAGGG |
| miR-146a | C | GTGCCGAGGAGGGATCTAGAA | CCTGCACGCTAACCCTCTCT |
| miR-145a | N | AGATTGCTTCCTGAGAGTAGACAACA | GTTAACTGAATTACTGGGTTGGAGC |
| miR-153 | C₁ | CAGAAACTGCACACCCACTCC | GCTGGTTCTCTACTGCCCCC |
| miR-153 | C₂ | GGGCTGCTGTGTTTACAACAAC | CAATCAGGGAGGAAACCGG |
| miR-153 | C₃ | CAAAGAGCAAGTTTAAAAGACCCC | GGTGGAAGGCCTGTCAAGAG |
| miR-153 | C₄ | ACAGGTTATTTGATAACCCAAGGAGA | GGAACCCGCTGACCTAGGA |
| miR-153 | C₅ | GTACCAGGGTCTGAGCCCAG | CATGGCCCTGTCTCCCAAC |
| miR-153 | C₄ | AGCAGCAGCCTCCCACAG | CGTGACTGGAGACCCAGTT |
| miR-153 | N | CTATGGACGCCCTGTGTGC | TTAGAGGCTTCAGCAGGCCA |
| miR-457/miR-195 | C₁ | GGCTTTGGGCGGGAGT | CTCTTCTGGGTCCTTGTAGGGAT |
| miR-457/miR-195 | C₂ | GCAGGACAATGGAAGGAAACC | GTACGGAGAGGGCGGATATG |
| miR-457/miR-195 | C₃ | AGGCCTTCCGACGACTCAG | GTTAGGGATATCGAGGTTGGCA |
| miR-457/miR-195 | C [symbol] | CCATCTGGAGAGCGAGGGA | GGGTGAACGCCTGGGTCT |
| miR-457/miR-195 | N | TCCGTCTTTTGCCTGCCTC | AAATTGCATCGGGACAGAG |
| let-7a-1/let-7f-1/let-7c | C | TCCGTCGCCATTTTATTTCG | CATTCTGCCCACCCGCT |
| let-7a-1/let-7f-1/let-7c | N | AGAAGTTTCCGATGAACATATGAAGA | AGCACTATGAGCCTTCTGACAT |
| let-7g | C | GTTTTCGCGGAACACCTTAGC | ACCGACAGCGTGTTGCG |
| let-7g | N | CTGTCGGGAAGTGAACACACC | CATGGACCAAAATATGGCATCAT |
| miR-99a/let-7 [symbol] /miR-125b-2 | C₁ | TGCACCTATTGTGTCCCTGC | ACAGTGGCCAATCGGCA |
| miR-99a/let-7 [symbol] /miR-125b-2 | C₂ | CACCCACTTCTTACCAAGAACTCC | GCTTTAAGTTGTTCACCCTCAAGTTA |
| miR-99a/let-7 [symbol] /miR-125b-2 | N | AGTTTCACTGCTTCATTCTAAATCCTG | CAATGTTTTCCATGTTGGATCAAA |
| CDKN1A(FIG. 2a) | | CAGATTTGTGGCTCAGTTCGTG | CCTGCGTTGGTGCGCT |
| negative(FIG. 2c) | | AAACCACCCATCGAGAAGGG | CGTGGCAGCACTCGTAAGACT |

| | | Genomic coordinates |
|---|---|---|
| miRNA transcription unit | Amplicon | (Human May 2004 assembly) |
| miR-15amiR-16- [symbol] | U | chr13: 49,555,159-49,555,239 |
| miR-15amiR-16- [symbol] | S | chr13: 49,554,223-49,554,273 |
| miR-15amiR-16- [symbol] | D | chr13: 49,553,109-49,553,159 |
| miR-22 | U | chr17: 1,565,542-1,555,592 |
| miR-22 | B | chr17: 1,566,424-1,555,474 |
| miR-22 | D | chr17: 1,557,075-1,557,129 |
| miR-26a-1 | D | chr2: 37,676,975-37,579,627 |
| miR-26a-2 | U | chr12: 56,527,775-56,527,825 |
| miR-26a-3 | [symbol] | chr12: 55,526,949-55,526,999 |
| miR-25b | U | chr2: 219,089,009-219,089,059 |
| miR-25b | [symbol] | chr2: 219,089,633-219,096,683 |
| miR-25b | D | chr2: 219,090,605-219,090,655 |
| miR-29b-1/miR-29a | C₁ | chr7: 130,055,217-130,055,267 |
| miR-29b-1/miR-29a | C₂ | chr7: 130,055,889-130,055,939 |
| miR-29b-1/miR-29a | N | chr7: 130,055,635-130,055,666 |
| miR-29b-2/miR-29c | C | chr1: 204,384,655-204,384,715 |
| miR-29b-2/miR-29c | N | chr1: 204,385,311-204,385,351 |
| miR-30a/miR-30c-2 | C₁ | chr5: 72,171,179-72,171,179 |
| miR-30a/miR-30c-2 | C₂ | chr5: 72,175,815-72,175,865 |
| miR-30a/miR-30c-2 | C₃ | chr5: 72,178,504-72,178,554 |
| miR-30a/miR-30c-2 | C₄ | chr6: 72,181,043-72,181,053 |
| miR-30a/miR-30c-2 | C₅ | chr5: 72,185,502-72,185,552 |
| miR-30a/miR-30c-2 | C₆ | chr6: 72,187,355-72,187,465 |
| miR-30d/miR-30b | O | chr6: 135,913,664-135,913,734 |
| miR-30d/miR-30b | N | chr6: 135,916,115-135,916,165 |
| miR-30e/miR-30c-1 | U | chr1: 40,825,582-40,825,632 |
| miR-30e/miR-30c-1 | [symbol] | chr1: 40,826,360-40,826,410 |
| miR-30e/miR-30c-1 | D | chr1: 40,827,102-40,827,157 |
| miR-34a | C₁ | chr1: 9,176,596-9,176,646 |
| miR-34a | C₂ | chr1: 9,176,405-9,176,456 |
| miR-34a | C₂ | chr1: 9,176,176-9,176,226 |
| miR-34a | N₁ | chr1: 9,192,066-9,192,116 |
| miR-34a | N₂ | chr1: 9,196,246-9,196,296 |
| miR-34a | N₂ | chr1: 9,196,970-9,197,020 |
| miR-146a | C | chr5: 159,827,695-159,927,745 |
| miR-145a | N | chr5: 159,824,570-159,825, [symbol] |
| miR-153 | C₁ | chr19: 54,595,108-54,595,158 |
| miR-153 | C₂ | chr19: 54,656,255-54,596,317 |
| miR-153 | C₃ | chr19: 54,656,411-54,596,461 |
| miR-153 | C₄ | chr19: 54,556,514-54,596,854 |
| miR-153 | C₅ | chr19: 54,595,881-54,595,932 |
| miR-153 | C₄ | chr19: 54,707,714-54,737,764 |
| miR-153 | N | chr19: 54,7 [symbol] ,550-54,700,711 |
| miR-457/miR-195 | C₁ | chr17: 6,866,331-6,855,381 |
| miR-457/miR-195 | C₂ | chr17: 6,866, [symbol] - [symbol] ,855, [symbol] |
| miR-457/miR-195 | C₃ | chr17: 6,866, [symbol] - [symbol] ,855, [symbol] |
| miR-457/miR-195 | C [symbol] | chr17: 6,866,951- [symbol] ,857,331 |
| miR-457/miR-195 | N | chr17: 5,953,862-6,853,912 |
| let-7a-1/let-7f-1/let-7c | C | chr9: 94, [symbol] 8,251-94, [symbol] ,301 |
| let-7a-1/let-7f-1/let-7c | N | chr5: 54, [symbol] ,470-54, [symbol] 6,520 |
| let-7g | C | chr3: 52,287,359-52,297,4 [symbol] |
| let-7g | N | chr3: 52,295,423-52,239,473 |
| miR-99a/let-7 [symbol] /miR-125b-2 | C₁ | chr21: 15,364,637-16,354,687 |
| miR-99a/let-7 [symbol] /miR-125b-2 | C₂ | chr21: 15,488,479-16,489,529 |
| miR-99a/let-7 [symbol] /miR-125b-2 | N | chr21: 15,487,995-16,489, [symbol] |
| CDKN1A(FIG. 2a) | | chr6: 36,754,186-36,754,236 |
| negative(FIG. 2c) | | chr1: 204,366,522-204,356,872 |

[symbol]

indicates data missing or illegible when filed

RACE Mapping of miRNA Primary Transcripts

The GeneRacer kit (Invitrogen) was used to characterize the miR-29b-2/29c, miR29b-1/29a, and miR-146a primary transcripts. Prior to isolating total RNA for use in these assays, Drosha expression was inhibited by electroporating previously described short-interfering RNAs (siRNAs) (Hwang Science 315, 97-100 (2007)) into tet-treated P493-6 cells. Electroporations were performed as described (O'Donnell et al., Mol Cell Biol 26, 2373-86 (2006)). Primer sequences are provided in Table 6 below.

TABLE 6
Primer sequences for characterization of the miR-29b-2/29c primary transcript

| | Forward primer sequence | Reverse primer sequence |
|---|---|---|
| Amplicon | (5′-3′) | (5′-3′) |
| 5′ RACE | CGACTGGAGCACGAGGACACTGA | GTCAACCCTCTGCATACCCATCTCC |
| 5′ nested RACE | GGACACTGACATGGACTGAAGGAGTA | ATAAAAAGTTTTGGGAGCCCTGAGC |
| 3′ RACE | AGAGCTGCTGCTGCTGATACTGC | GCTGTCAACGATACGCTACGTAACG |
| 3′ nested RACE | TGGGGACAACAGATTTGCATTGA | CGCTACGTAACGGCATGACAGTG) |

Primer sequences for characterization of the miR-29b-1/29a primary transcript

| | Forward primer sequence | Reverse primer sequence |
|---|---|---|
| Amplicon | (5′-3′) | (5′-3′) |
| 5′ RACE | CGACTGGAGCACGAGGACACTGA | TCCAAGAACTCACACATTCAGGCAAA |
| 5′ nested RACE | GGACACTGACATGGACTGAAGGAGTA | GTCTGCCGTGACAGTTCAGTAGGAG |
| 3′ RACE | CTCCTACTGAACTGTCACGGCAGAC | GCTGTCAACGATACGCTACGTAACG |
| 3′ nested RACE | GTATGGATTCATTGCCAGGAGCTG | CGCTACGTAACGGCATGACAGTG |

Primer sequences for characterization of the miR-146a primary transcript

| | | |
|---|---|---|
| | Forward primer sequence | Reverse primer sequence |
| Amplicon | (5′-3′) | (5′-3′) |
| 5′ RACE | CGACTGGAGCACGAGGACACTGA | GCTGAGGATACACATCGGCTTTTC |
| 5′ nested RACE | GGACACTGACATGGACTGAAGGAGTA | CTCCTCGTTGTGCTACTGTCTCCTG |
| 3′ RACE | TTCAGCTGGGATATCTCTGTCATCG | GCTGTCAACGATACGCTACGTAACG |
| 3′ nested RACE | GGGCTTGAGGACCTGGAGAGAGT | CGCTACGTAACGGCATGACAGTG) |

Primer sequences for miRNA cloning

| miRNA | Forward primer sequence | Reverse primer sequence |
|---|---|---|
| transcription unit | (5′-3′) | (5′-3′)) |
| miR-15a/miR-16-1 | ACCGCTCGAGGGCACAGAATGGACTTCAG | ATACCGCTCGAGATGGCTTTTCCCCTTCAGAT |
| miR-22 | ACCGCTCCAGCATGCCCTGTCAGATCTTT | ATACCGCTCGAGCTCTCCAACTTGCCCAAAAC |
| miR-26a-2 | ATACCGCTCGAGCGGCAGGGTGTCTGTCTAGT | ATACCGCTCGAGCAGGCTTCCAATGGATCAGT |
| miR-29b-1/miR-29a | ACCGCTCGAGGCATGCTCTCCCATCAATA | ATACCGCTCGAGACCACATGCAATTCAGGTCA |
| miR-30b | ATACCGCTCGAGGATCCTGAATGCTGTGCCTGTTCTTT | ATACCGCTCGAGATCCCTGCCAGCTAGACAA |
| miR-34a | ATACCGCTCGAGCCTCCTGCATCCTTTCTTT | ATACCGCTCGAGCCTGTGCCTTTTTCCTTCC |
| miR-146a | ATACCGCTCGAGAGATCCACCCACATCAGC | ATACCGCTCCAGCCTGAGACTCTGCCTTCTG |
| miR-150 | ATACCGCTCGAGGAGTGGGTGTGCAGTTTCT | ATACCGCTCGAGAGCGCACCAGAGGATATGT |
| miR-195/miR-497 | ATACCGCTCGAGTCCCCTGAGCTGAGTTCCTA | ATACCGCTCGAGATTTCCCTCTCAGCTTCGTG |
| let-7a-1/let-7f-1 | ATACCGCTCGAGGAGCGGATTCAGATAACCA | ATACCGCTCGAGCAGGACCTGACCTTGGACAT |

### Tumorigenesis Assays

The miRNAs and at least 100 bp of flanking sequence were amplified from genomic DNA and cloned into the XhoI site of the retroviral vector MSCV-PIG⁴¹. Primer sequences are provided in Table 6. Correct vector construction was verified by direct sequencing. Retroviral infection of Myc3 and 38B9 cells, flow cytometry, and tumor formation were performed as described (Yu et al., Ann N Y Acad Sci 1059, 145-59 (2005)). The sequence of the inserts are provided below.

has-miR-15a/16-1
CTCGAGGGCACAGAATGGACTTCAGTTAAGTTTTTGATGTAGAAATGTTTTATTATTCTACTTAAAATCTCCTTA
AAAATAATTATGCATATTACATCAATGTTATAATGTTTAAACATAGATTTTTTTACATGCATTCTTTTTTTCCTG
AAAGAAAATATTTTTTATATTCTTTAGGCGCGAATGTGTGTTTAAAAAAAATAAAACCTTGGAGTAAAGTAGCAG
CACATAATGGTTTGTGGATTTTGAAAAGGTGCAGGCCATATTGTGCTGCCTCAAAAATACAAGGATCTGATCTTC
TGAAGAAAATATATTTCTTTTTATTCATAGCTCTTATGATAGCAATGTCAGCAGTGCCTTAGCAGCACGTAAATA
TTGGCGTTAAGATTCTAAAATTATCTCCAGTATTAACTGTGCTGCTGAAGTAAGGTTGACCATACTCTACAGTTG
TGTTTTAATGTATATTAATGTTACTAATGTGTTTTCAGTTTTATTGATAGTCTTTTCAGTATTATTGATAATCTT
GTTATTTTTAGTATGATTCTGTAAAAATGAATTAATACTAATTTTTCAGATGTATCATCTCTTAAAATACTGTAA
TTGCAATTTAATAATTGTATTGAATGCCATCAAGTTTTTTTAAAAAGCTTATGCAGCATTAGAGGAATTTATTTT
AATGCACATTTATATTCAACATAGACATTAATTCAGATTTTTACTTGGGATAAAACAAATTCTAGTTTTCCCTTT
GTTTTGAAATTACTTTTAAAATATGTCTTTACAGATAAATATAAAATATATTAAGCATTTTGAACAGAGCTTAGA
AGACAATATTTAGTACTGTTTCTGAATATTTCTTTATATCTGAAGGGGAAAAGCCATCTCGAG
has-miR-18a
CTCGAGCCTCGGGAAGCCAAGTTGGGCTTTAAAGTGCAGGGCCTGCTGATGTTGAGTGCTTTTTGTTCTAAGGTG
CATCTAGTGCAGATAGTGAAGTAGATTAGCATCTACTGCCCTAAGTGCTCCTTCTGGCATAAGAAGTTATGTATT
CATCCAATAATTCAAGCCAAGCAAGTATATAGGTGTTTTAATAGTTTTTGTTTGCAGTCCTCTGCTCGAG
has-miR-22
CTCGAGCATGCCCTGCTCAGATCTTTCCCATTTTCCCTCCCTTTCCCTTAGGAGCCTGTTCCTCTCACGCCCTCA
CCTGGCTGAGCCGCAGTAGTTCTTCAGTGGCAAGCTTTATGTCCTGACCCAGCTAAAGCTGCCAGTTGAAGAACT
GTTGCCCTCTGCCCCTGGCTTCGAGGAGGAGGAGGAGCTGCTTTCCCCATCATCTGGAAGGTGACAGAAATGGGC
TGGGAAGGTCCGAACAGCAGGGTGGATGATACGTTTTGGGCAAGTTGGAGAGCTCGAG
has-miR-26a-2
CTCGAGCGGCAGGGTGTCTGTCTAGTCTATGGTCATTGAGGGGAAAAAGTCACTTCTCCCTGGTGCAATTCATTA
CCTAATCATGACCTGGACAGACTGTCCTGTCGGAGCCAAGGACAGAAAGCTCCCATAGAGGCTGTGGCTGGATTC
AAGTAATCCAGGATAGGCTGTTTCCATCTGTGAGGCCTATTCTTGATTACTTGTTTCTGGAGGCAGCTGATGGTC
CGCCGCCGGAAACAGAGATGGCTCCTGGGACATGGTGTGTGCGCTTCTTCCTGAGCCAGGTTGAGGTTGGGACCA
CTGATCCATTGGAAGCCTGCTCGAG
has-miR-29b-1/29a
CTCGAGGCATGCTCTCCCATCAATAACAAATTCAGTGACATCAGTTTATGAATATATGAAATTTGCCAAAGCTCT
GTTTAGACCACTGAGTAACTCACAGCTAGGTTTCAACTTTTCCTTTCTAGGTTGTCTTGGGTTTATTGTAAGAGA
GCATTATGAAGAAAAAAATAGATCATAAAGCTTCTTCAGGAAGCTGGTTTCATATGGTGGTTTAGATTTAAATAG
TGATTGTCTAGCACCATTTGAAATCAGTGTTCTTGGGGGAGACCAGCTGCGCTGCACTACCAACAGCAAAAGAAG
TGAATGGGACAGCTCTGAAGTATTTGAAAGCAACAGCAGGATGGCTGTGAGAACCTGCCTCACATGTAGCTGACC
CCTTCCTCACCCCTGCCAACAGTGGTGGCATATATCACAAATGGCAGTCAGGTCTCTGCACTGGCGGATCCAACT
GTGATCGAAAGTTTTCCAAAAATAAGTTGTGTCTGTATTGAACATGAACAGACTTTCTTCTTGTCATTATTCTCT
AACAATACTGCATAACAATTATTTGCATACATTTGCATTGCATTAAGTATTCTAAGTAATCTAGAGACGATTTAA
AGTATACGGGAGGATGTGTGTAGGTTGTATGCAAATACTACACCATTTTCTATCAGAGACTTGAGCATCTGTGGA
TTTTGGTATCCAAGGGGCTTTCTGGAACCAATCCCTCAAGGATACCAAGGGATGAATGTAATTGTACAGGATATC
GCATTGTTGGAATTTTATACTTCTTTGTGGAATAAACCTATAGCACTTAATAGATAGTACAGACTCATTCCATTG
TGCCTGGGTTAAAGAGCCCAATGTATGCTGGATTTAGTAAGATTTGGGCCCTCCCAACCCTCACGACCTTCTGTG
ACCCCTTAGAGGATGACTGATTTCTTTTGGTGTTCAGAGTCAATATAATTTTCTAGCACCATCTGAAATCGGTTA
TAATGATTGGGGAAGAGCACCATGATGCTGACTGCTGAGAGGAAATGTATTGGTGACCGTTGGGGCCATGGACAA
GAACTAAGAAAACAAATGCAAAGCAATAATGCAAAGGTGATTTTTCTTCTTCCAGTTTCTAAGTTGAATTTCACT
GACCTGAATTGCATGTGGTCTCGAG
has-miR-30b
CTCGAGGATCCTGAATGCTGTGCCTGTTCTTTTTTTCAACAGAGTCTTACGTAAAGAACCGTACAAACTTAGTAA
AGAGTTTAAGTCCTGCTTTAAACCAAGTTTCAGTTCATGTAAACATCCTACACTCAGCTGTAATACATGGATTGG
CTGGGAGGTGGATGTTTACTTCAGCTGACTTGGAATGTCAACCAATTAACATTGATAAAAGATTTGGCAAGAATA
GTATACAGAGGCTTGAATTTTTAATGTAATTAATGTAATTAAAGGTTTGTTGGAAATGTGAGACCATTTTGTTCT
CCCAGAGAAAAAGTGTGTTAATTGTCTAGCTGGCAGGGATCTCGAG
has-miR-34a
CTCGAGCCTCCTGCATCCTTTCTTTCCTCCCCACATTTCCTTCTTATCAACAGGTGCTGGGGAGAGGCAGGACAG
GCCTGTCCCCCGAGTCCCCTCCGGATGCCGTGGACCGGCCAGCTGTGAGTGTTTCTTTGGCAGTGTCTTAGCTGG
TTGTTGTGAGCAATAGTAAGGAAGCAATCAGCAAGTATACTGCCCTAGAAGTGCTGCACGTTGTGGGGCCCAAGA
GGGAAGATGAAGCGAGAGATGCCCAGACCAGTGGGAGACGCCAGGACTTCGGAAGCTCTTCTGCGCCACGGTGGG
TGGTGAGGGCGGCTGGGAAAGTGAGCTCCAGGGCCCCAGGAGCAGCCTGCTCGTGGGTGCGGAAGGAAAAAGGCA
CAGGCTCGAG
has-miR-146a
CTCGAGAGAGATCCACCCACATCAGCCTTCCAGACTGCTGGCCTGGTCTCCTCCAGATGTTTATAACTCATGAGT
GCCAGGACTAGACCTGGTACTAGGAAGCAGCTGCATTGGATTTACCAGGCTTTTCACTCTTGTATTTTACAGGGC
TGGGACAGGCCTGGACTGCAAGGAGGGGTCTTTGCACCATCTCTGAAAAGCCGATGTGTATCCTCAGCTTTGAGA
ACTGAATTCCATGGGTTGTGTCAGTGTCAGACCTGTGAAATTCAGTTCTTCAGCTGGGATATCTCTGTCATCGTG
GGCTTGAGGACCTGGAGAGAGTAGATCCTGAAGAACTTTTTCAGTCTGCTGAAGAGCTTGGAAGACTGGAGACAG
AAGGCAGAGTCTCAGGCTCGAG
has-miR-150
CTCGAGGAGTGGGTGTGCAGTTTCTGCGACTCAGGGTGGCGTCCCCCCAACCTGTCCCTGCCCCTTCCTGCCCTC
TTTGATGCGGCCCCACTTCCTCTGGCAGGAACCCCCGCCCTCCCTGGACCTGGGTATAAGGCAGGGACTGGGCCC
ACGGGGAGGCAGCGTCCCCGAGGCAGCAGCGGCAGCGGCGGCTCCTCTCCCCATGGCCCTGTCTCCCAACCCTTG
TACCAGTGCTGGGCTCAGACCCTGGTACAGGCCTGGGGGACAGGGACCTGGGGACCCCGGCACCGGCAGGCCCCA
AGGGGTGAGGTGAGCGGGCATTGGGACCTCCCCTCCCTGTACTCCCATCTCTGCTGCGGCTTTTATGCGTCTCTC
CCCTTCGGGTCCCACATATCCTCTGGTGCGCTCTCGAG
has-miR-497/195
CTCGAGTCCCCTGAGCTGAGTTCCTACAGAGGGAAGATGGTCCAATCTTACTACACTGTGAGCTCATCCCCATGG
TCCGTCGCCTTCCAGTTGCCTGCTCAGCCCGTCCCTGGTTCCTCCCAAACGTTTTTGGGGGCCATGTTTGCCTTT
TAAGGCTTCTCTATCCCCCCGCTCCTGGAGGTGGTGCTGGGGTCTTCCCAGCACTGCTATGTGCTCTCTTCCTTT
CAACCCACCCCGGTCCTGCTCCCGCCCCAGCAGCACACTGTGGTTTGTACGGCACTGTGGCCACGTCCAAACCAC
ACTGTGGTGTTAGAGCGAGGGTGGGGGAGGCACCGCCGAGGCTTGGCCCTGGGAGGCCATCCTGGAGAAGTGACA
CAAAAAACATCTGGGGCCTTGTGACAAACTTCTTGCCAGGTGGGCAAGGAGAGGGTGGGGTATGTAAGCACCCCT
CTAAAATCTCCAGGGCAGTTTCAAGAATACTGATGGCCAGAGACCCTGGGAGTAAGTTCTGCCTCAAGAGAACAA
AGTGGAGTCTTTGTTGCCCACACCCAGCTTCCCTGGCTCTAGCAGCACAGAAATATTGGCACAGGGAAGCGAGTC
TGCCAATATTGGCTGTGCTGCTCCAGGCAGGGTGGTGAAAACTACCGAGGAGGGGCTGAGCCCCCATGGGCCGAG
GAGAGAAGAGGGAACAGGCCTCTCCTGCTAATAATGTTAAGCAGACAGCACGAAGCTGAGAGGGAAATCTCGAG
has-let-7a-1/7f-1
CTCGAGGAGCGGATTCAGATAACCAAGCATTTAAAATACTATTAATGAAATACAGGAAATGAAACCACAGCATAG
ATTATGCATGTAGCCAAAATGTTCAGTTAAACTTCATTTTCAACGTAAGTGAATGAAAATGGTCTAATACTATTT
TTCTTATCACTCACACAGGAAACCAGGATTACCGAGGAGGAAAAAAAGCCTTCCTGTGGTGCTCAACTGTGATTC
CTTTTCACCATTCACCCTGGATGTTCTCTTCACTGTGGGATGAGGTAGTAGGTTGTATAGTTTTAGGGTCACACC
CACCACTGGGAGATAACTATACAATCTACTGTCTTTCCTAACGTGATAGAAAAGTCTGCATCCAGGCGGTCTGAT
AGAAAGTCAGTTAACTAATTGTACAATATTTAAGATTAACTTGTCTTAAAGAGATGTAGTGCAGCATTTGTTTAT
GGCCTGGAAATAAATTAATTTAGAGATAAAGTCTGTAGCAAGTACACTGGATGGGGGTGGGGAAACCTTTTGCTT
CTTGTCTTATTTCTCTGTGTCAGAATAAATGTATTTTTTTATTTTGATTTATGCTGATAATTTTATGTTGAAATT
TTCTTTCGAAAGAGATTGTACTTTCCATTCCAGAAGAAAACATTGCTCTATCAGAGTGAGGTAGTAGATTGTATA
GTTGTGGGGTAGTGATTTTACCCTGTTCAGGAGATAACTATACAATCTATTGCCTTCCCTGAGGAGTAGACTTGC
TGCATTATTTTCTTTTTATTTAGATGATATTAAAACTCAGAAGAATTAATTTTGACATTTTGTATTTACAGTTTA
TCAGTTAATTTTCTCTGTTCAAGTAGTACAGTAGGCACAGATTAACATTTAAATTTTTCACATATGGTATATTTC
AGAAATTTGAAGTTAAGCAAAAATTTTAATGAGTAGAGAAAGTAAGTAGCCTTCAGGAAATCTTCATAGAGGACC
AGGCCCTTTTGGAATTGTGAATAGGTTTATTGCCTTACATCCTGGTACACATGTCCAAGGTCAGGTCCTGCTCGAG

### Accession Numbers

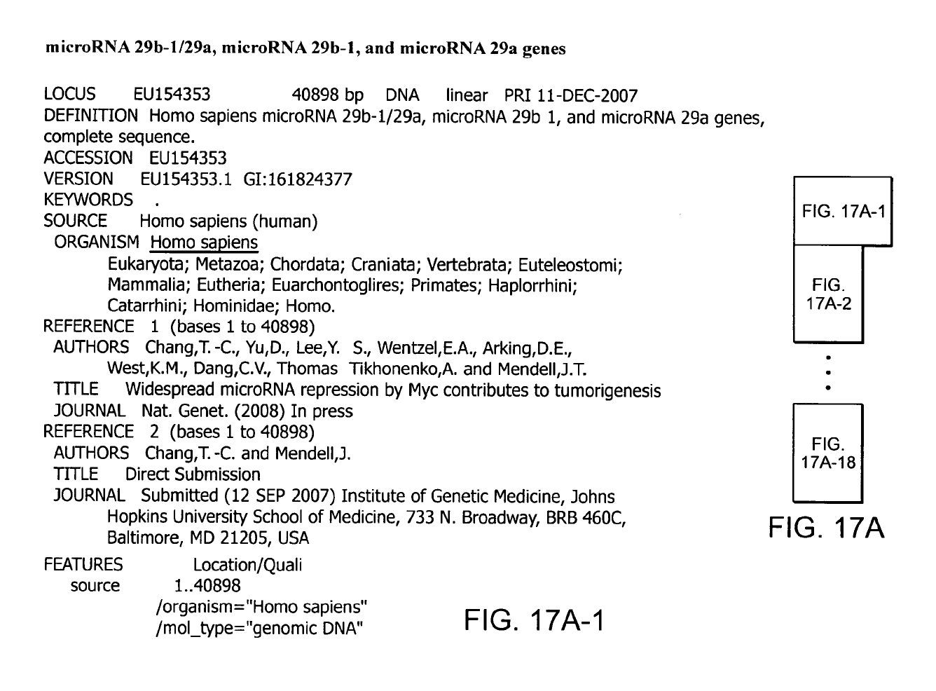

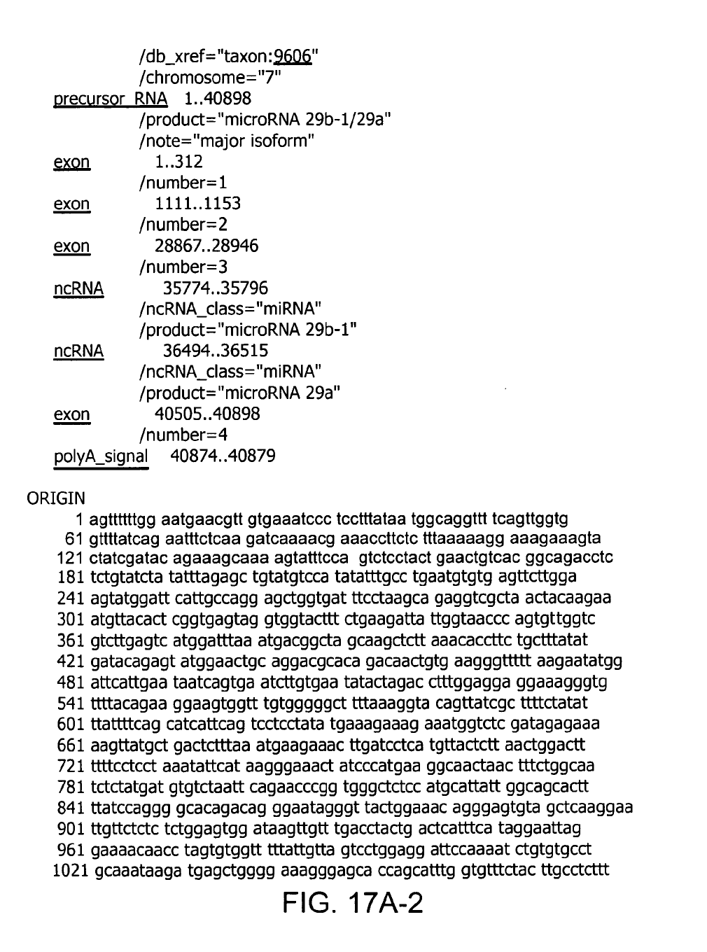

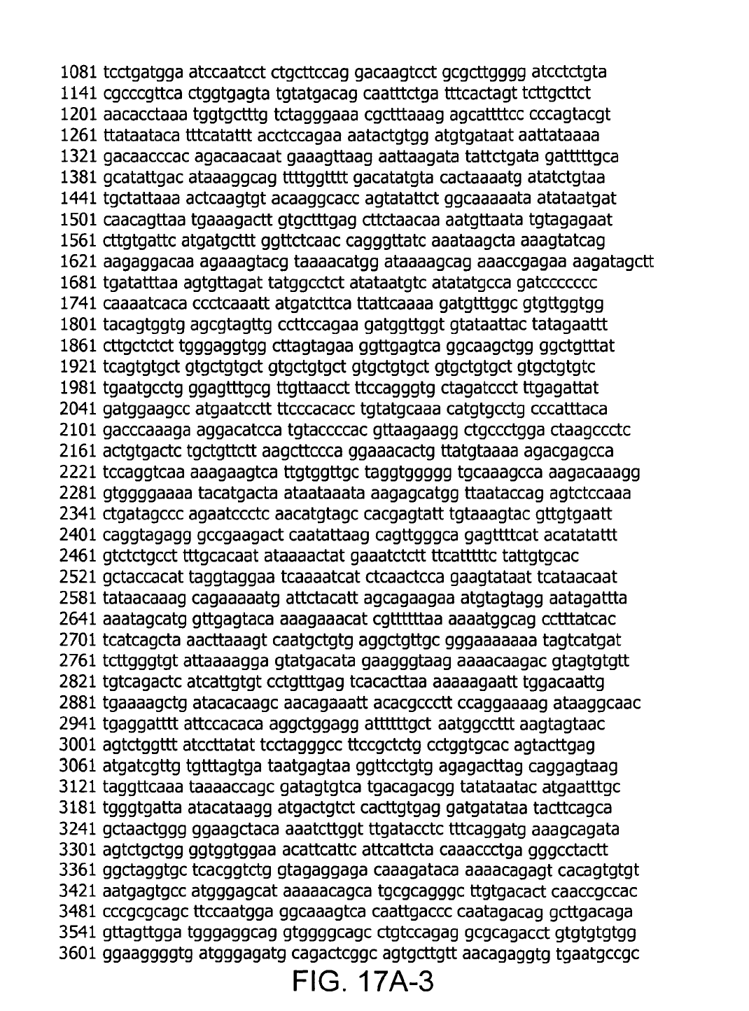

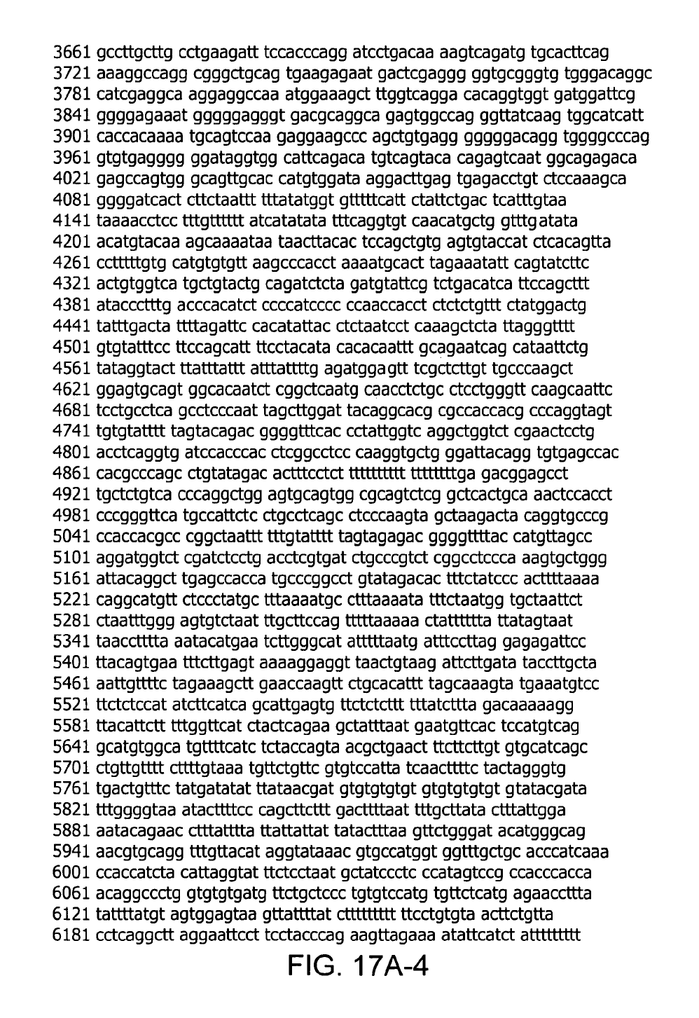

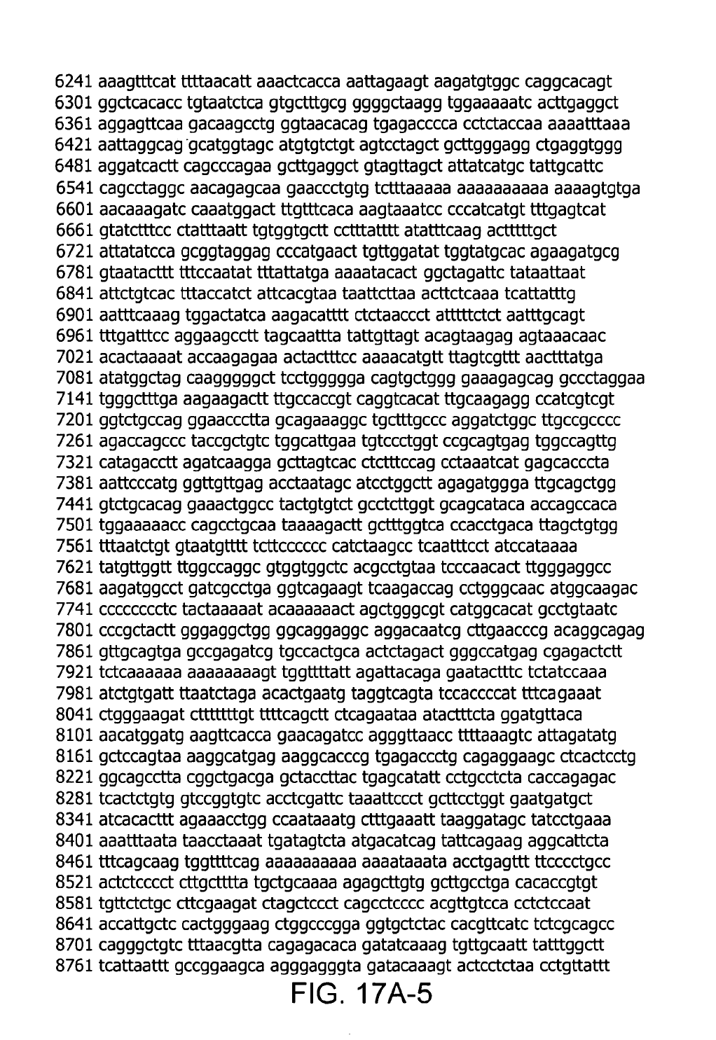

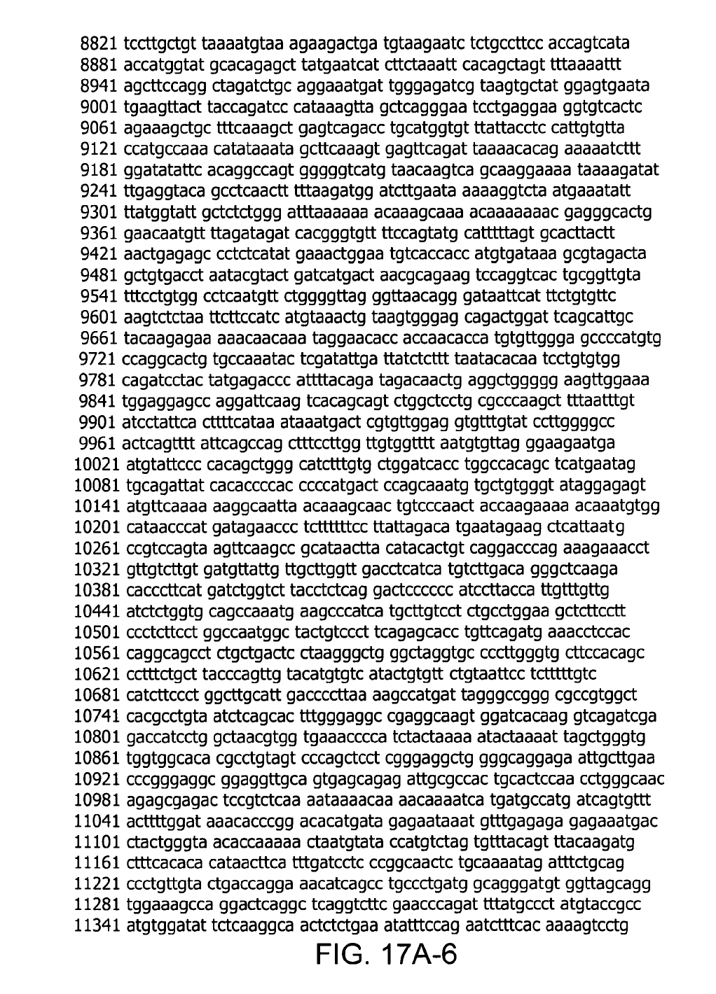

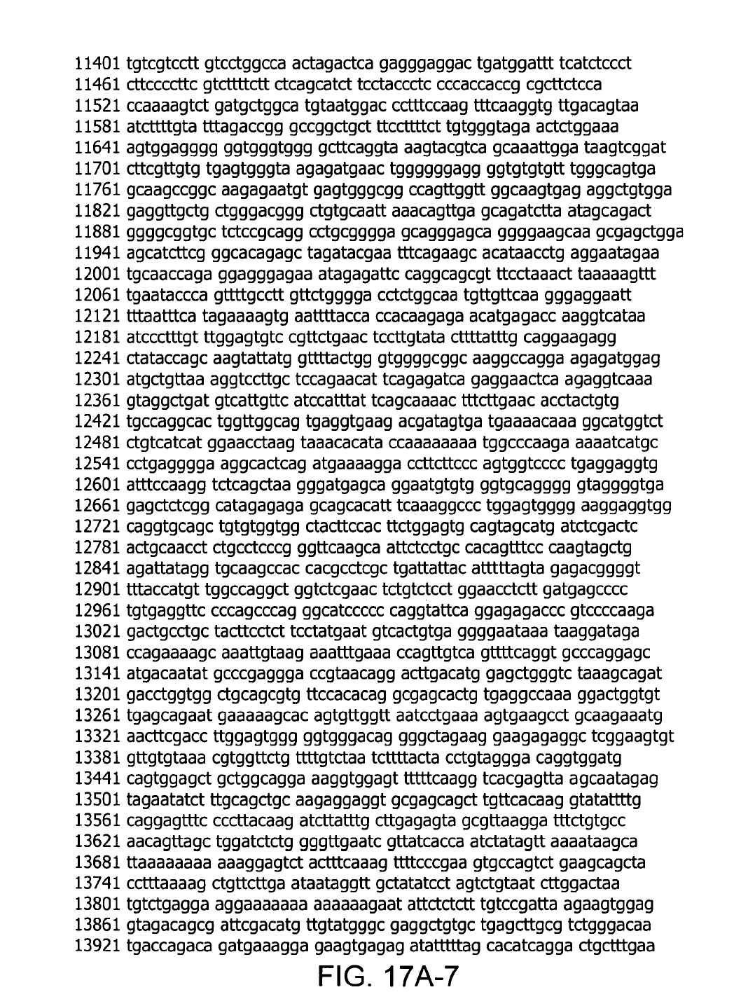

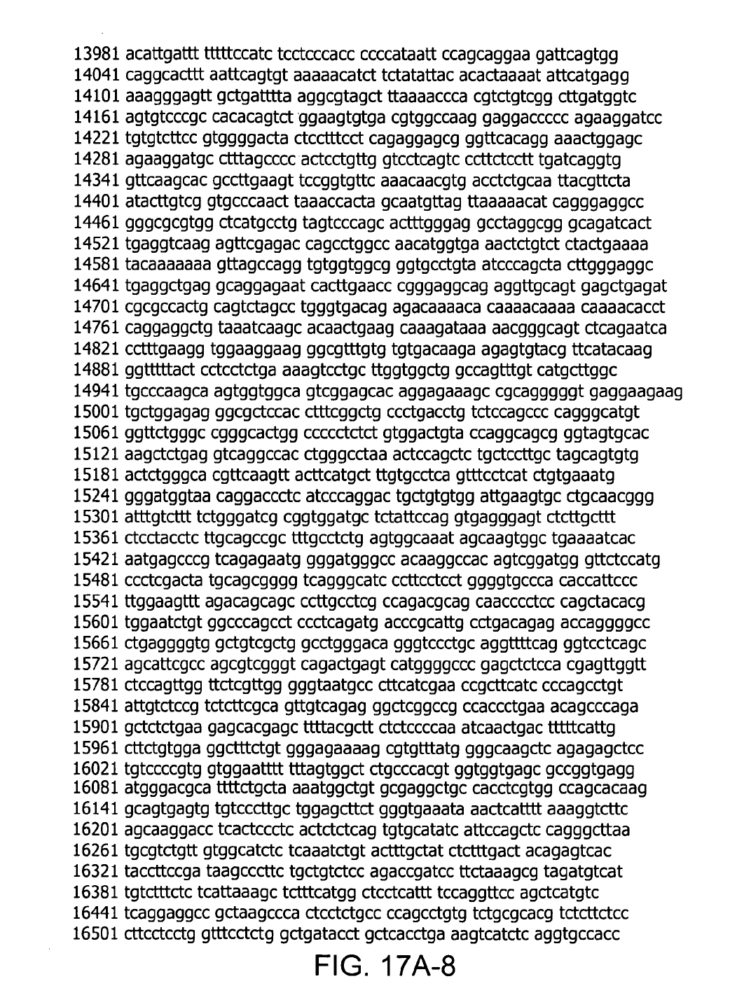

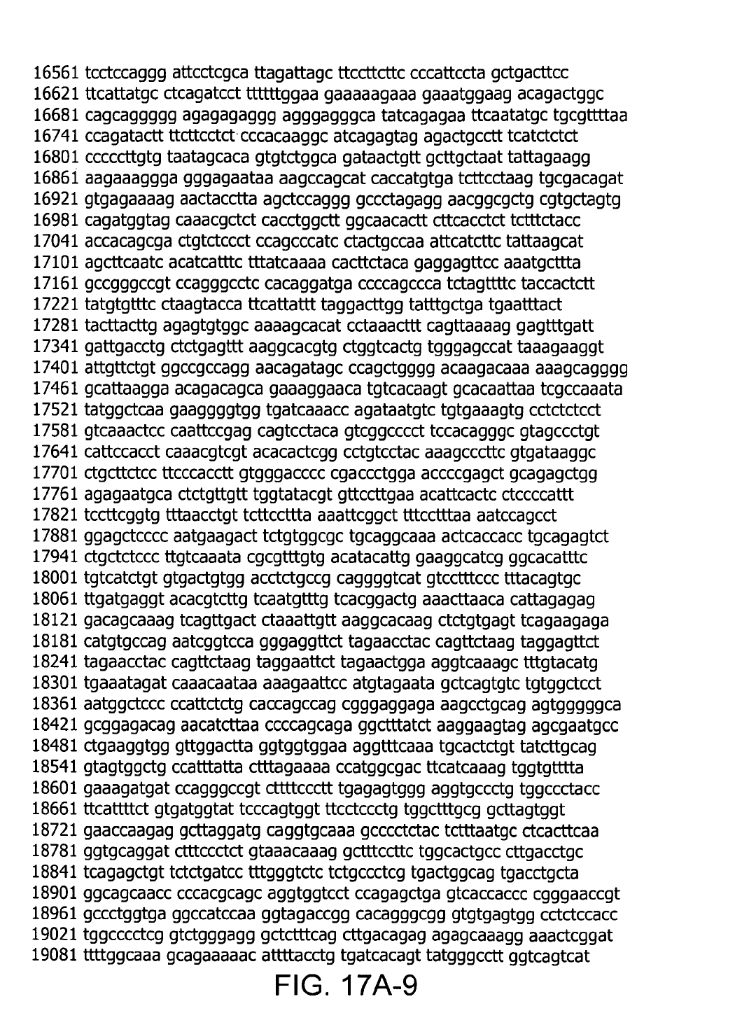

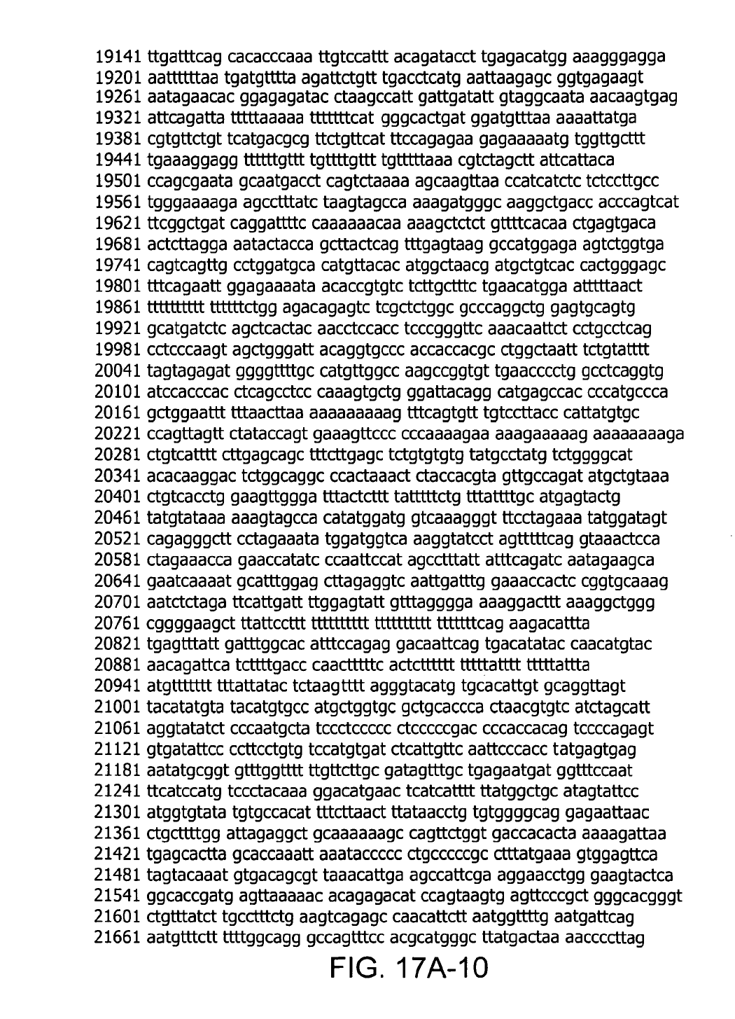

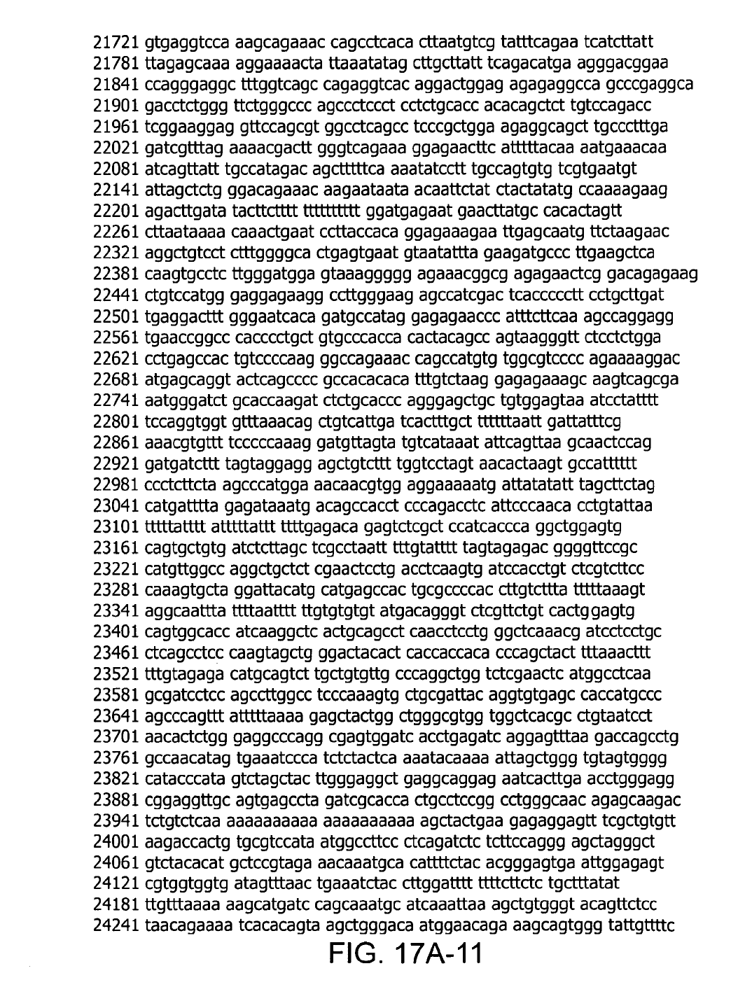

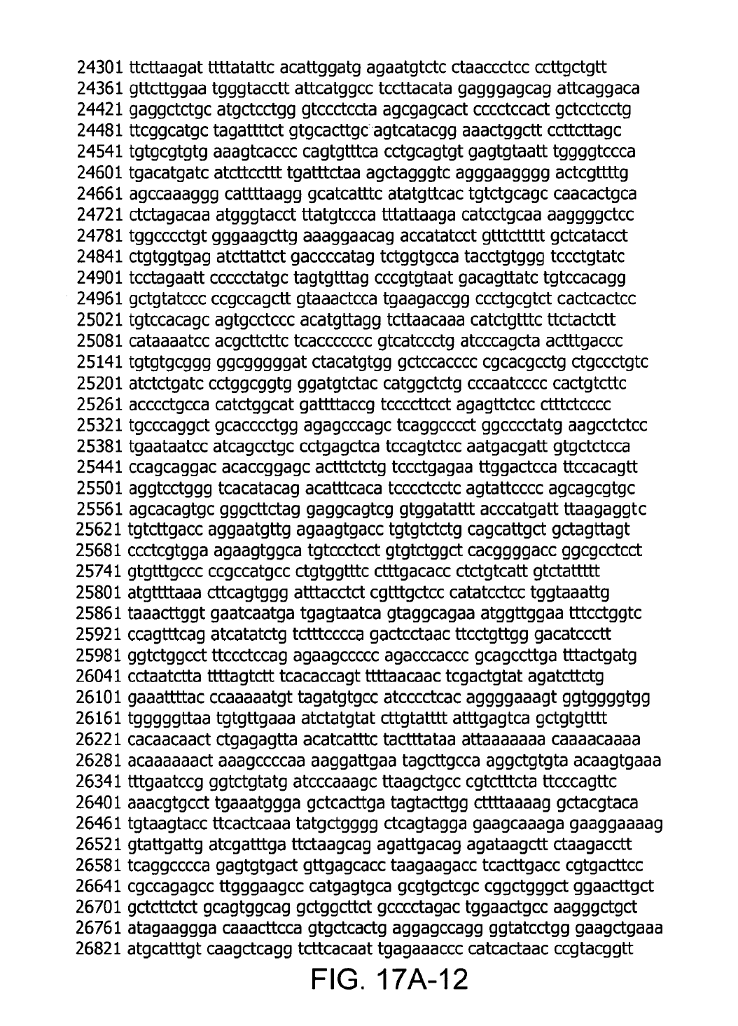

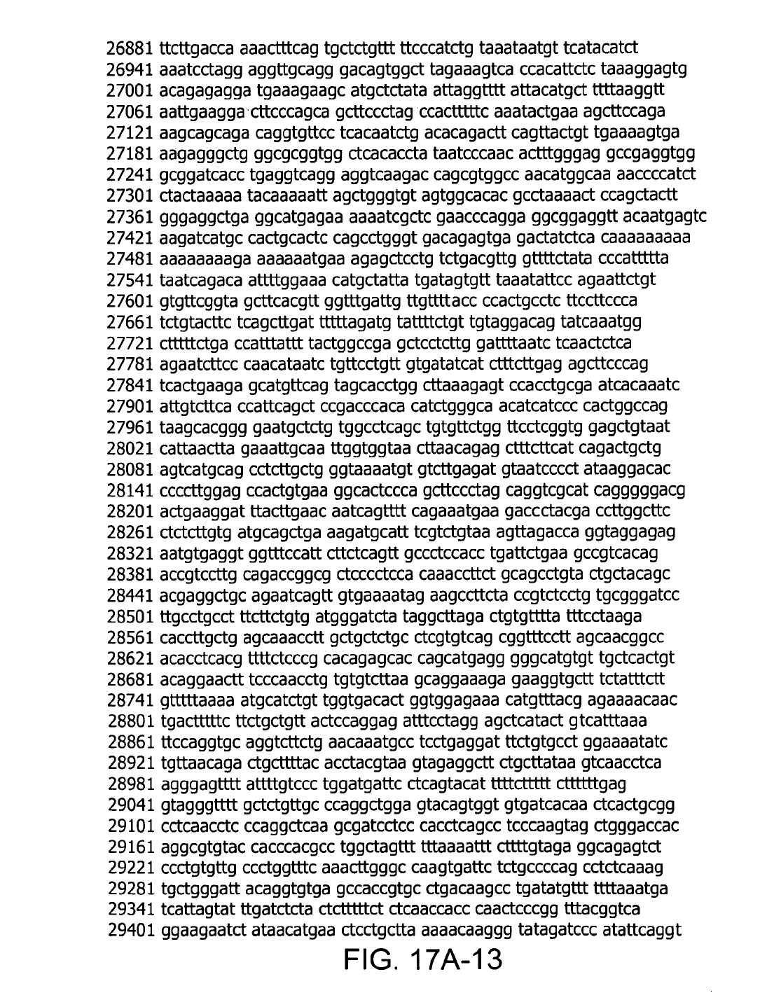

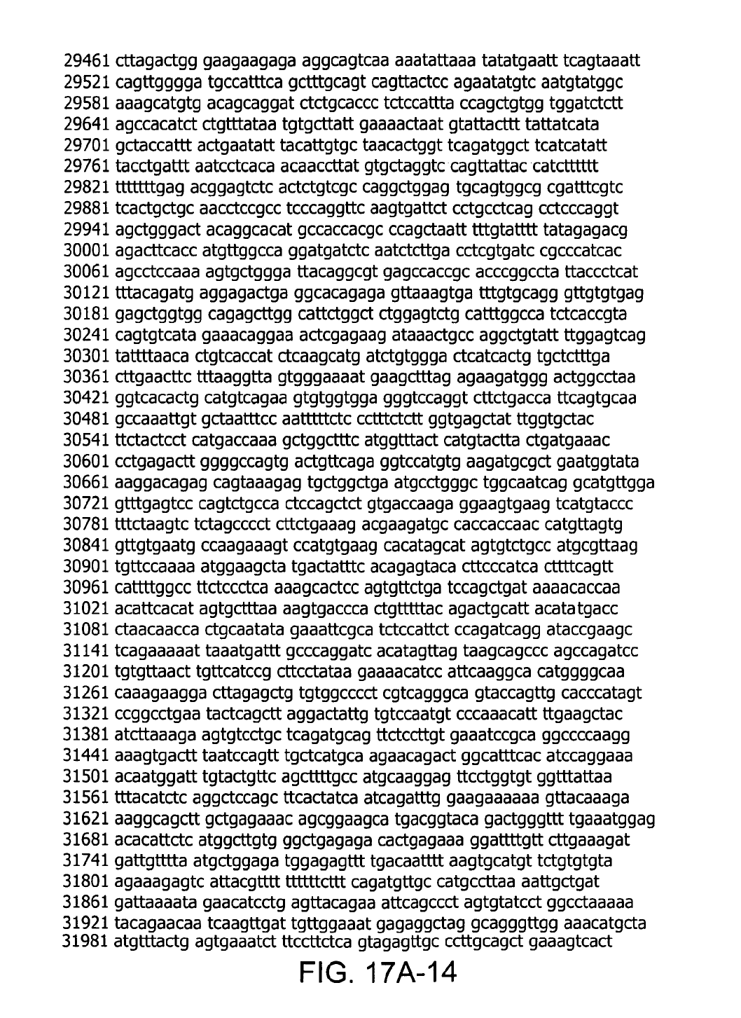

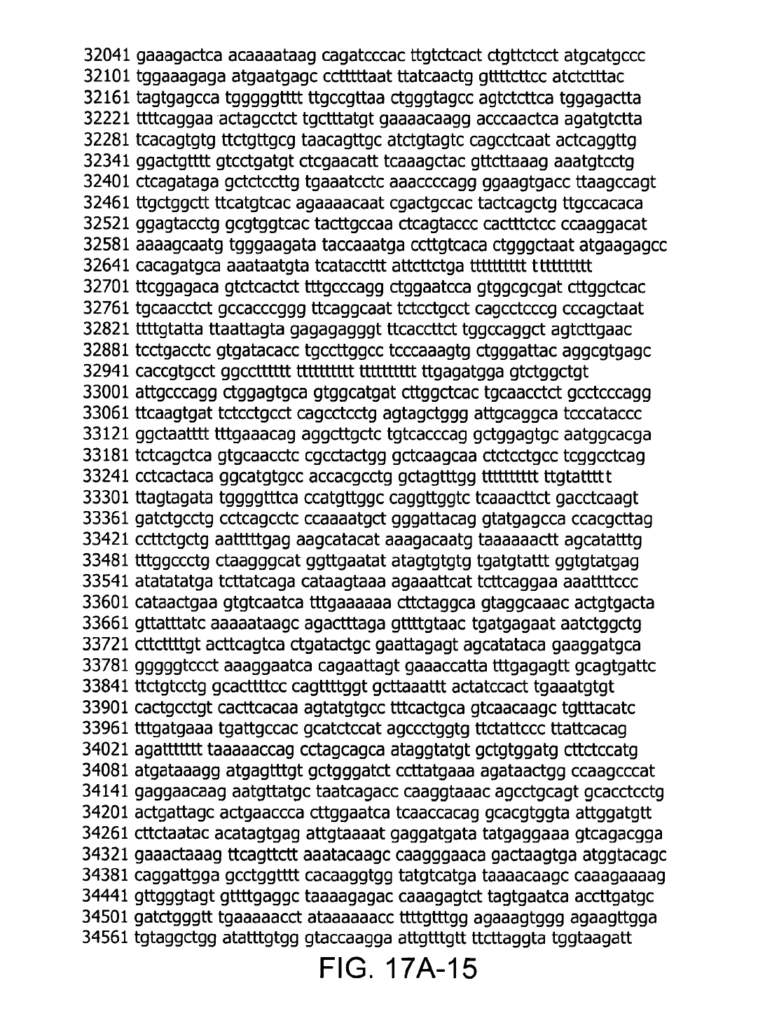

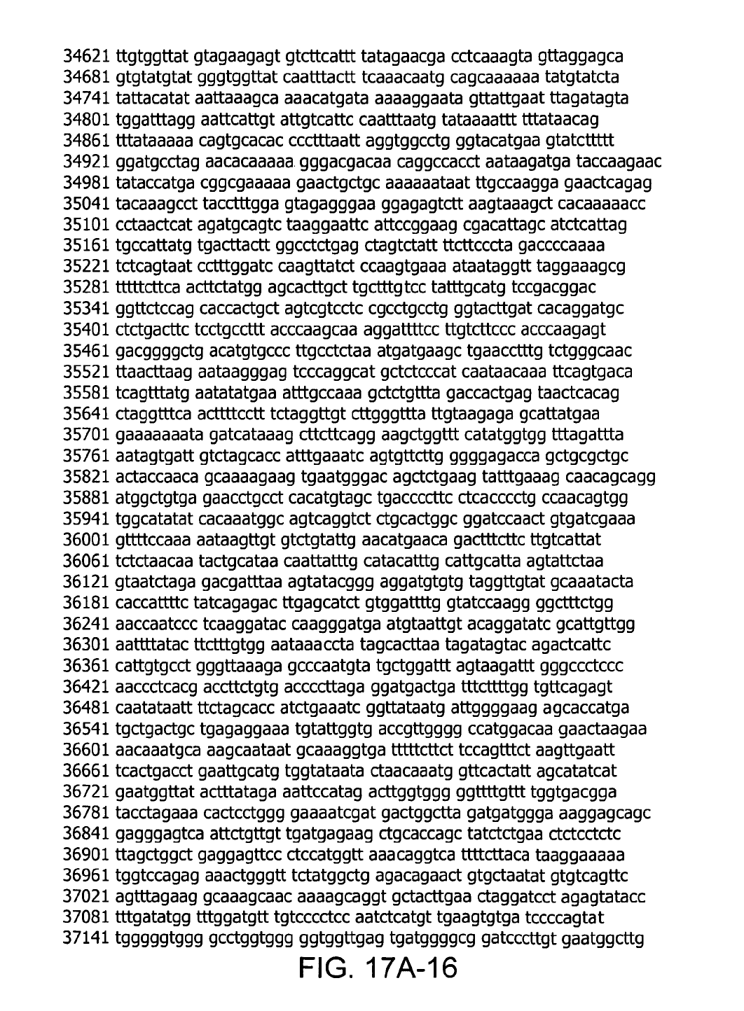

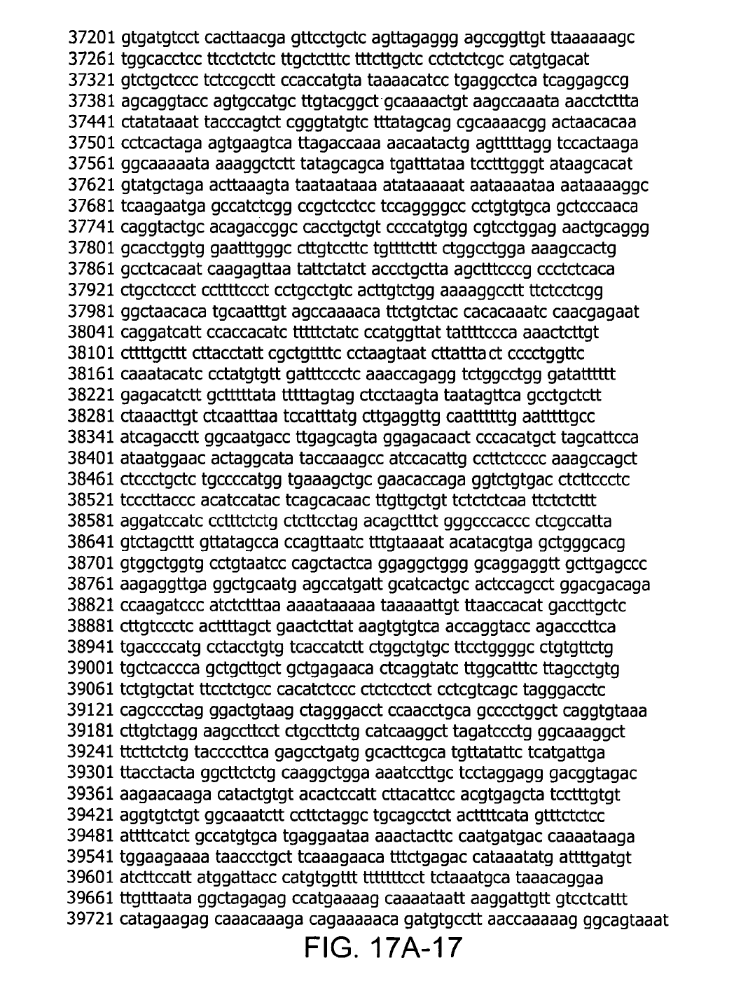

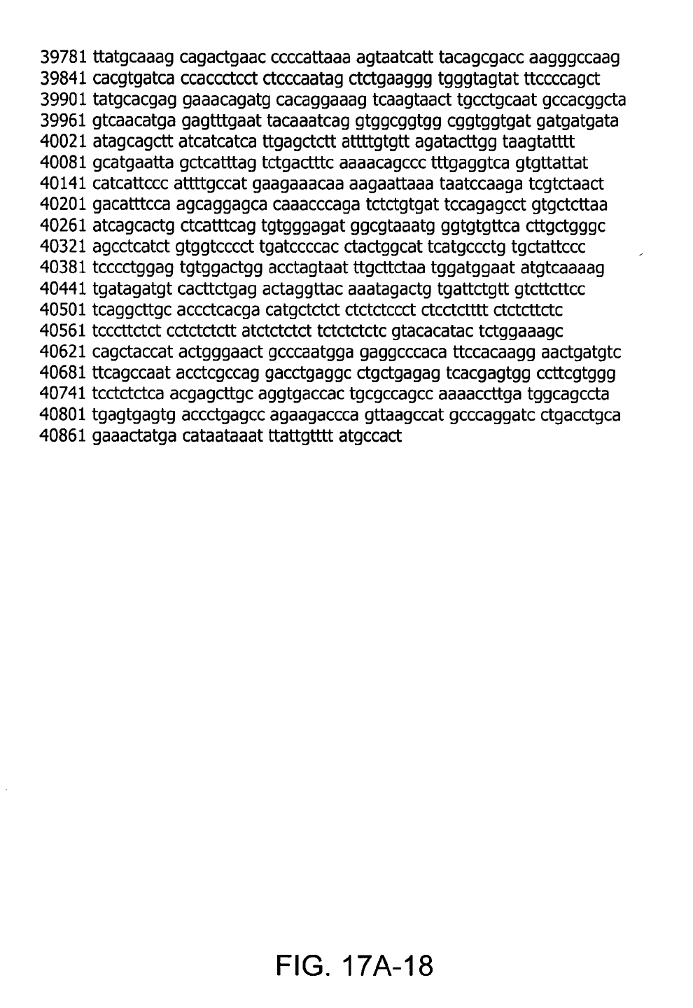

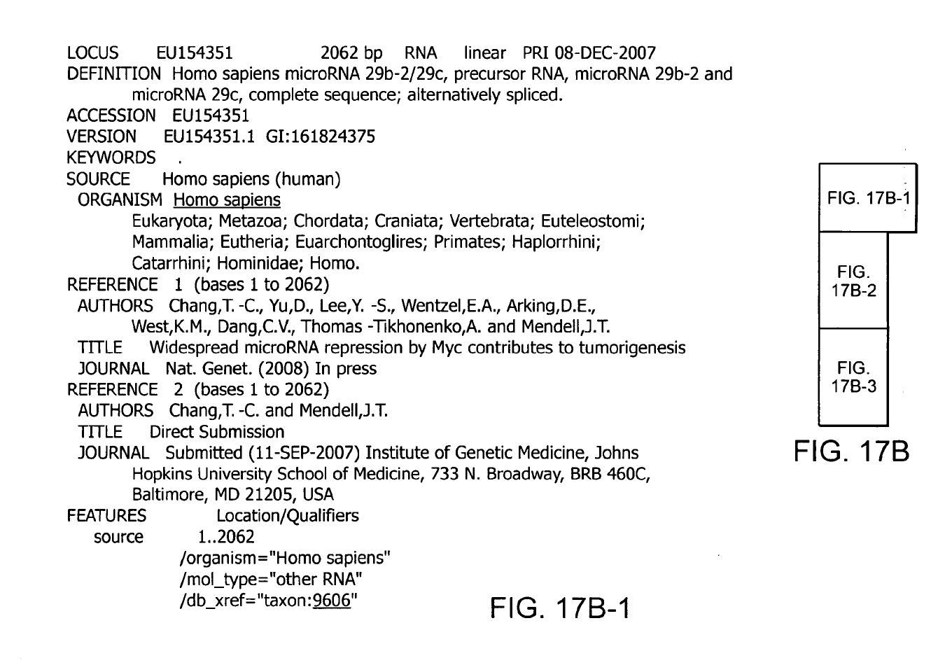

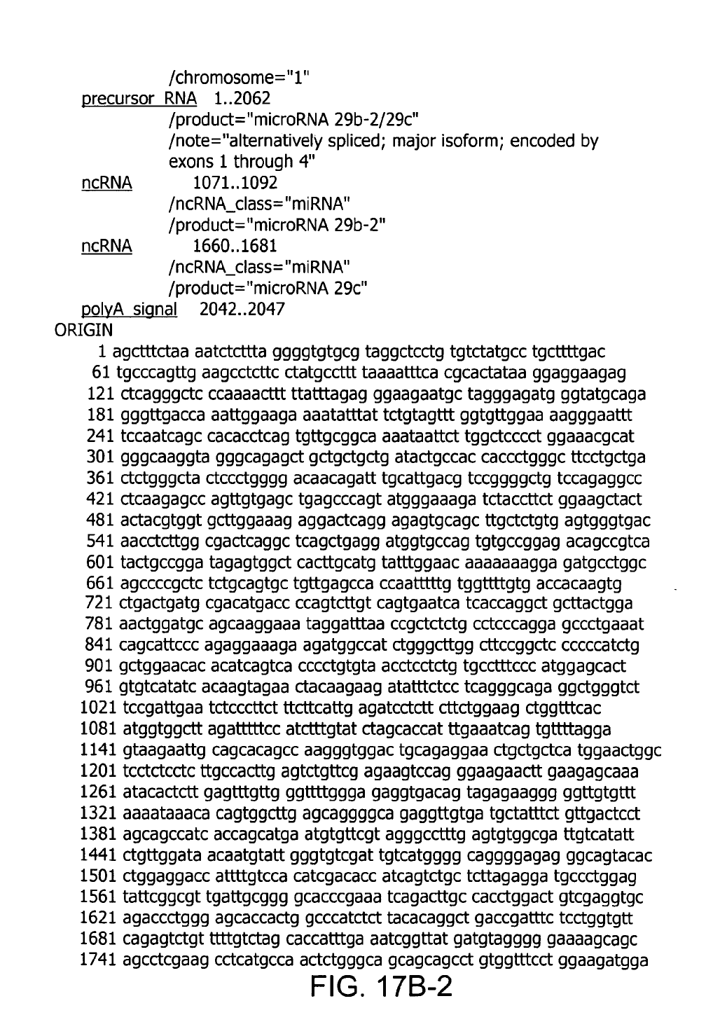

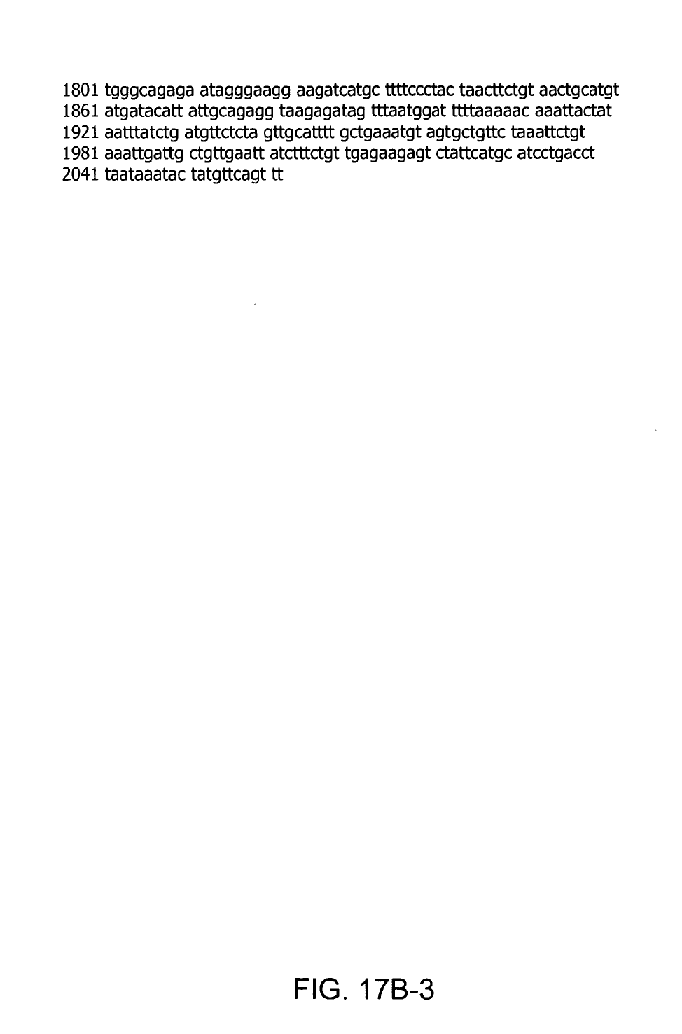

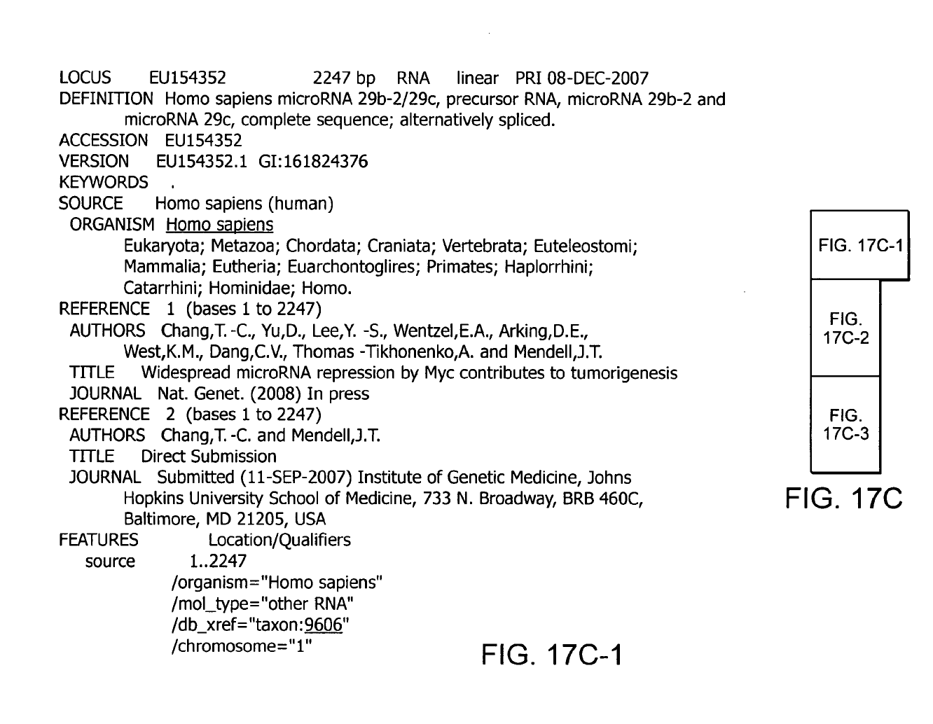

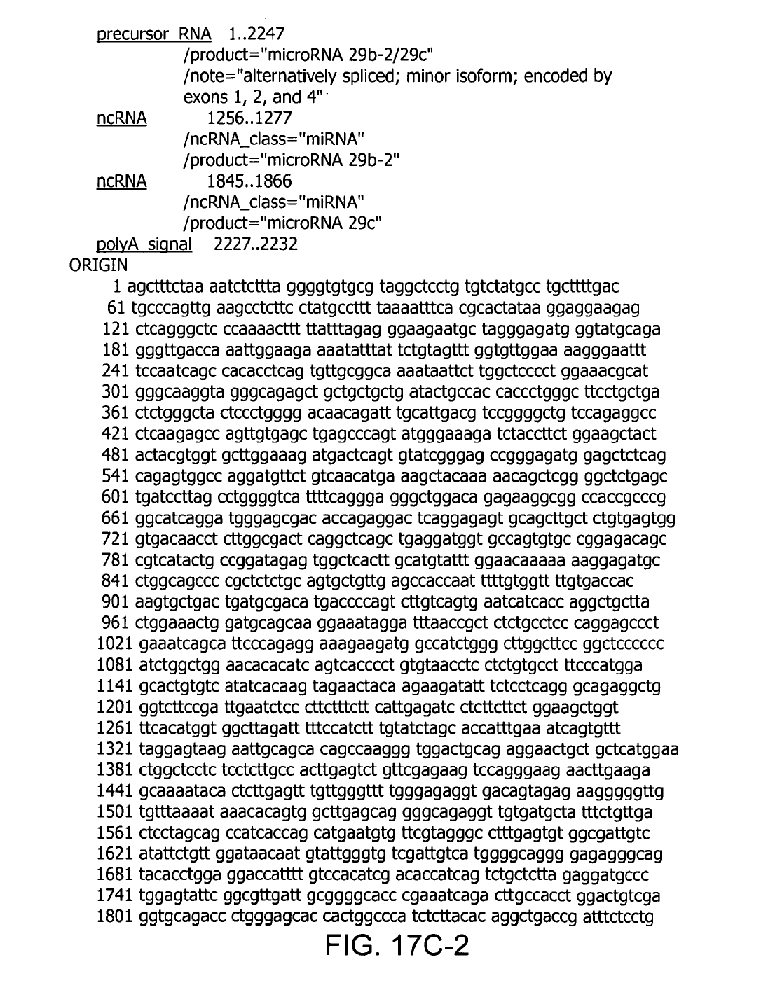

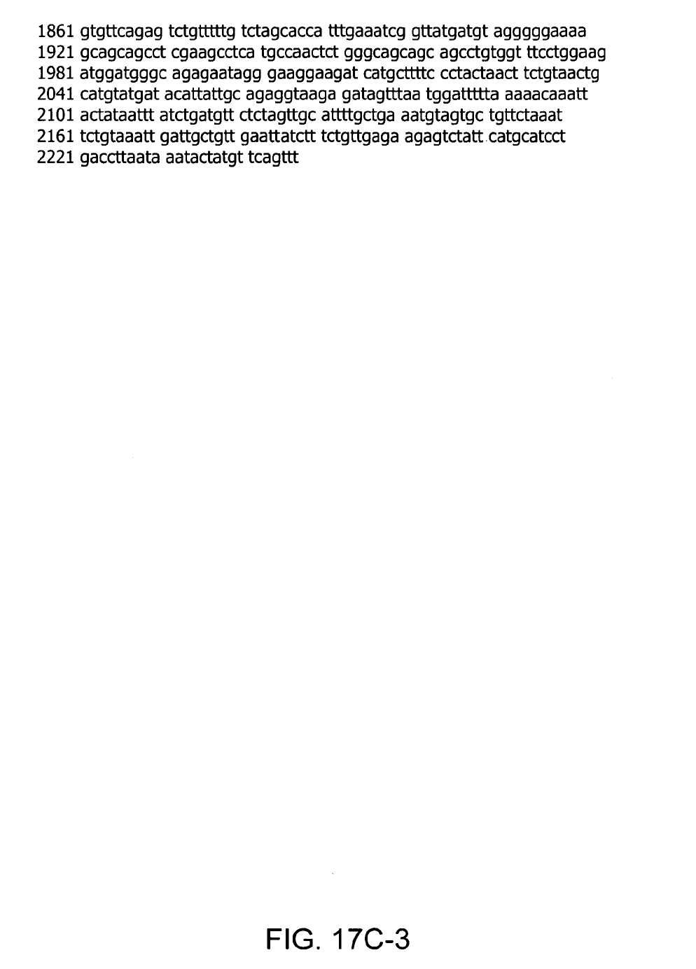

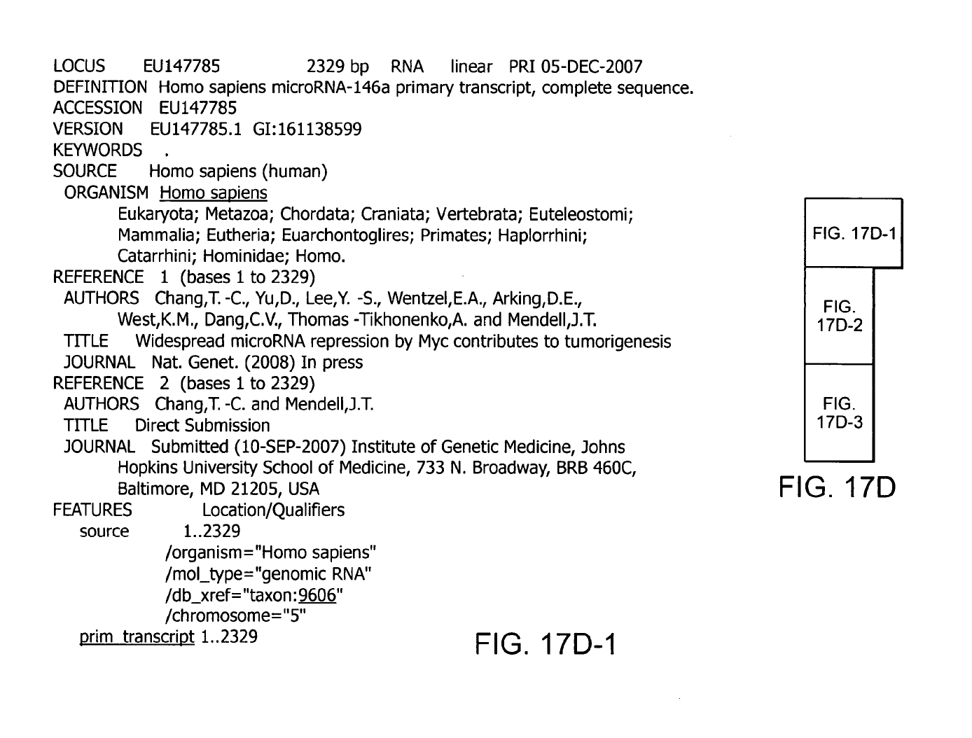

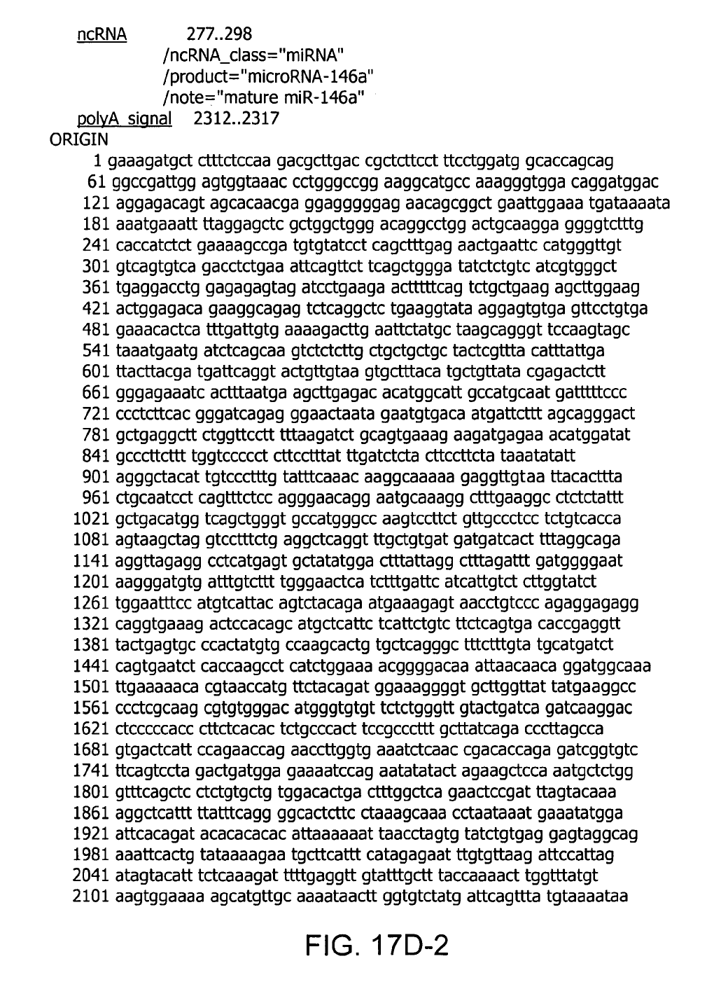

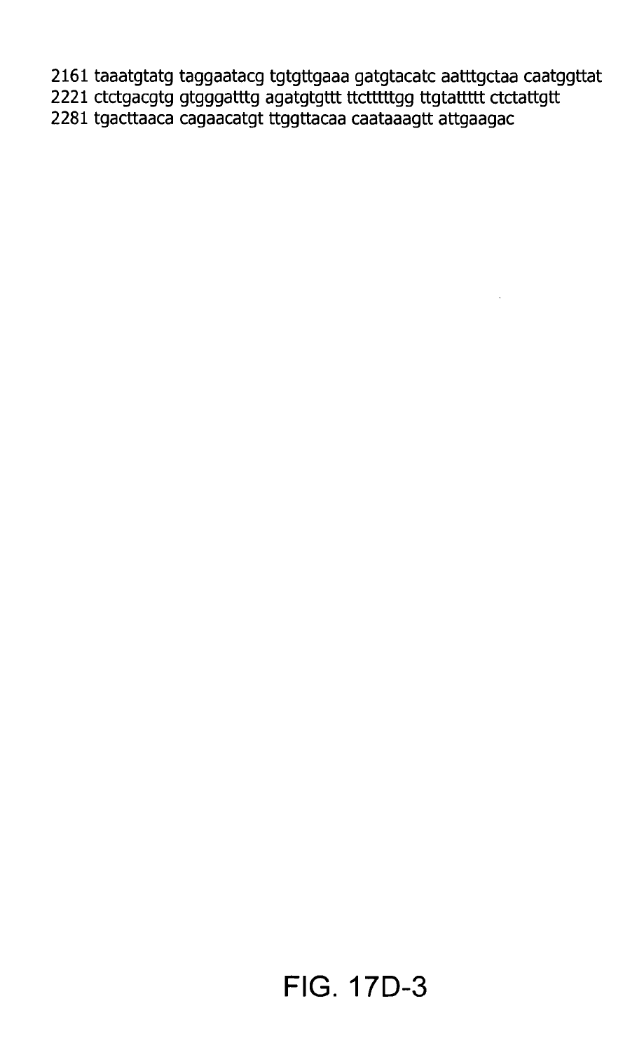

The sequences of miRNA primary transcripts have been deposited in the GenBank database under the following accession numbers: miR-29b-1/29a cluster, EU154353; miR-29b-2/29c cluster, EU154351, EU154352; miR-146a, EU147785 (FIG. 17A-E, respectively). Microarray data have been deposited in the Gene Expression Omnibus (GEO) database under accession number GSE9129.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. An isolated oligonucleotide comprising a nucleobase sequence having at least 85% identity to the sequence of a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1 or a fragment thereof, wherein expression of said microRNA in a neoplastic cell reduces the survival of the cell or reduces cell division.

2. The isolated oligonucleotide of claim 1, wherein said oligonucleotide comprises the nucleobase sequence of said microRNA.

3. The isolated oligonucleotide of claim 1, wherein said oligonucleotide consists essentially of the nucleobase sequence of said microRNA.

4. The isolated oligonucleotide of claim 1, wherein said microRNA sequence is a mature or hairpin form.

5. The isolated oligonucleotide of claim 1, wherein said oligonucleotide comprises at least one modified linkage.

6. The isolated oligonucleotide of claim 5, wherein said modified linkage is selected from the group consisting of phosphorothioate, methylphosphonate, phosphotriester, phosphorodithioate, and phosphoselenate linkages.

7. The isolated oligonucleotide of claim 5, wherein said oligonucleotide comprises at least one modified sugar moiety or one modified nucleobase.

8. An isolated nucleic acid molecule encoding the oligonucleotide of any of claims 1-4, wherein expression of the oligonucleotide in a neoplastic cell reduces the survival of the cell or reduces cell division.

9. The isolated nucleic acid molecule of claim 8, said nucleic acid molecule consisting essentially of the nucleotide sequence encoding a mature or hairpin form of a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1 or a fragment thereof.

10. An expression vector encoding an oligonucleotide of any one of claims 1-9, wherein the nucleic acid molecule is positioned for expression in a mammalian cell.

11. The expression vector of claim 10, wherein the vector encodes a microRNA selected from the group consisting of miR-22, miR-26a, miR-34a, miR-150, miR-195/497, and miR-15a/16-1.

12. The expression vector of claim 10, wherein the vector is a viral vector selected from the group consisting of a retroviral, adenoviral, lentiviral and adeno-associated viral vector.

13. A host cell comprising the expression vector of claim 8 or the oligonucleotide of any one of claims 1-4.

14. A pharmaceutical composition for the treatment of a neoplasia, the composition comprising an effective amount of an oligonucleotide having at least 85% identity to the sequence of a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1 and a pharmaceutically acceptable excipient, wherein expression of said microRNA in a neoplastic cell reduces the survival of the cell or reduces cell division.

15. The pharmaceutical composition of claim 13, wherein the oligonucleotide has at least 95% identity to said microRNA.

16. The pharmaceutical composition of claim 14, wherein the amount of microRNA is sufficient to reduce cell survival, cell proliferation, or expression of Myc in a neoplastic cell by at least about 5% relative to an untreated control cell.

17. The pharmaceutical composition of claim 14, wherein the composition comprises at least one of miR-22, miR-26a, miR-34a, miR-150, miR-195/497, or miR-15a/16-1.

18. A pharmaceutical composition for the treatment of a neoplasia, the composition comprising an effective amount of an expression vector encoding a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1 and a pharmaceutically acceptable excipient, wherein expression of said microRNA in a neoplastic cell reduces the survival of the cell or reduces cell division.

19. The pharmaceutical composition of claim 18, wherein the amount of microRNA is sufficient to reduce expression of Myc in a neoplastic cell by at least about 5% relative to an untreated control cell.

20. The pharmaceutical composition of claim 14 or 18, wherein the composition comprises at least one of miR-22, miR-26a, miR-34a, miR-150, miR-195/497, or miR-15a/16-1.

21. The pharmaceutical composition of claim 14 or 18, wherein the composition comprises two, three, four, five, or six microRNAs selected from the group consisting of miR-22, miR-26a, miR-34a, miR-150, miR-195/497, and miR-15a/16-1.

22. The pharmaceutical composition of claim 14, wherein the oligonucleotide comprises a modification.

23. A method of reducing the growth, survival or proliferation of a neoplastic cell, the method comprising contacting the cell with an oligonucleotide comprising a nucleobase sequence having at least 85% identity to a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1, thereby reducing the growth, survival or proliferation of a neoplastic cell relative to an untreated control cell.

24. A method of reducing the growth, survival or proliferation of a neoplastic cell, the method comprising contacting the cell with an expression vector encoding a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1, thereby reducing the growth, survival or proliferation of a neoplastic cell relative to an untreated control cell.

25. The method of claim 23, wherein the cell is a mammalian cell.

26. The method of claim 23, wherein the cell is a human cell.

27. The method of claim 24, wherein the cell is a lymphoma cell.

28. The method of any one of claims 23-27, wherein the method induces apoptosis in the neoplastic cell.

29. A method of treating neoplasia in a subject, the method comprising administering to the subject an effective amount of an oligonucleotide comprising a nucleobase sequence having at least 85% identity to a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1, thereby treating a neoplasia in the subject.

30. A method of treating neoplasia in a subject, the method comprising administering to the subject an effective amount of an expression vector encoding a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1, thereby treating the neoplasia in the subject.

31. The method of claim 29, wherein the oligonucleotide comprises a modification that enhances nuclease resistance.

32. The method of any one of claims 22-30, wherein the subject is diagnosed as having a lymphoma.

33. The method of any one of claims 22-30, wherein the method induces apoptosis in a neoplastic cell of the subject.

34. The method of any one of claims 22-30, wherein the effective amount is sufficient to reduce expression of Myc in a neoplastic cell by at least about 5% relative to an untreated control cell.

35. The method of any one of claims 22-28, wherein the subject is contacted with two, three, four, five, or six microRNAs selected from the group consisting of miR-22, miR-26a, miR-34a, miR-150, miR-195/497, and miR-15a/16-1.

36. A method of characterizing a neoplasia, the method comprising assaying the expression of a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1.

37. The method of claim 36, wherein the method comprises assaying the expression of a combination of microRNAs consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1.

38. The method of claim 36, wherein the neoplasia is characterized as having Myc disregulation.

39. A method of identifying an agent for the treatment of a neoplasia, the method comprising
(a) contacting a neoplastic cell with a candidate agent; and
(b) assaying the expression of a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, and miR-15a/16-1, wherein an increase in said microRNA expression identifies the agent as useful for the treatment of a neoplasia.

40. The method of claim 39, further comprising testing the agent in a functional assay.

41. The method of claim 39, wherein the functional assay analyses cell growth, proliferation, or survival.

42. A primer set comprising at least two pairs of oligonucleotides, each of which pair binds to a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1 or a fragment thereof.

43. A probe set comprising at least two oligonucleotides each of which binds to a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1 or a fragment thereof.

44. A microarray comprising a microRNA or nucleic acid molecule encoding a microRNA selected from the group consisting of miR-22, miR-26a-1, miR-26a-2, miR-29b-2, miR-29c, miR-30e, miR-30c-1, miR-146a, miR-150, let-7a-1, let-7f-1, let-7d, miR-100, let-7a-2, miR-125b-1, let-7a-3, let-7b, miR-99a, let-7c, miR-125b-2, miR-99b, let-7e, miR-125a, let-7f-2, miR-98, let-7g, let-7i, miR-26b, miR-30c, miR-34a, miR-150, miR-195/497, miR-15a/16-1 or a fragment thereof.
